(19) United States
(12) Patent Application Publication
(10) Pub. No.: US 2020/0256862 A1
(43) Pub. Date: Aug. 13, 2020

(54) FUNCTIONALIZED SOLID SUPPORT

(71) Applicant: Massachusetts Institute of Technology, Cambridge, MA (US)

(72) Inventors: Alexander K. Shalek, Cambridge, MA (US); Andrew Navia, Cambridge, MA (US); Jeffrey F. van Humbeck, Cambridge, MA (US); Marc H. Wadsworth II, Cambridge, MA (US); Shaina Carroll, Cambridge, MA (US); Travis K. Hughes, Cambridge, MA (US)

(21) Appl. No.: 16/758,504

(22) PCT Filed: Oct. 23, 2018
(86) PCT No.: PCT/US18/57173

§ 371 Date: Apr. 23, 2020

Related U.S. Application Data
(60) Provisional application No. 62/575,883, filed on Oct. 23, 2017.

(51) Int. Cl. G01N 33/543; C12Q 1/6816

(52) U.S. Cl. CPC G01N 33/54313; C12Q 1/6816; G01N 33/54326

(57) ABSTRACT

The present invention relates to functionalized solid supports and methods of making functionalized solid supports. Methods for preparing a population of high quality functionalized solid supports for use in various nucleic acid analysis methods are provided. The invention also provides methods for validation and quality control of the functionalization steps.

### CROSS REFERENCE TO RELATED APPLICATIONS

This application claims the benefit of U.S. Provisional Application No. 62/575,883, filed Oct. 23, 2017. The entire contents of the above-identified applications are hereby fully incorporated herein by reference.

### REFERENCE TO AN ELECTRONIC SEQUENCE LISTING

The contents of the electronic sequence listing (BROD_2840WP_ST25.txt”; Size is 4 Kilobytes and it was created on Oct. 17, 2018) is herein incorporated by reference in its entirety.

### TECHNICAL FIELD

The present invention relates to a population of functionalized solid support and a method for functionalizing and/or preparing a solid support.

### BACKGROUND

Capture beads have been extensively used in single cell sequencing methods. The efficiency of the methods depend, in part, on the quality and coupling efficiency of the beads. A major limitation of commercially-available capture beads include batch-to-batch variability in quality, i.e., the quality of the beads is variable and inconsistent. In addition, current quality control (QC) protocols, if any, fail to robustly characterize and validate synthesis batches. To overcome these limitations, Applicants have developed methods to improve the syntheses of the beads and novel quality control methods to robustly characterize and validate the syntheses of capture beads to ensure that the quality of the beads is consistent and that the beads produced are of a high-quality product. The quality of the beads provides Applicants the ability to confirm oligo sequence in a more efficient manner.

The methods developed by Applicants provide increases in the quality of the individual beads, i.e., higher percentage of functionalization, increase in the number of reactive sites, higher oligo density, higher transcript capture efficiency, increase in oligo sequence consistency in terms of identity and length of sequences, and monodisperity, i.e., particle size uniformity.

The methods provide a consistent quality of the beads across the bead population. The methods provide a reduction in bead-to-bead variability due to within lot or between lot variability in the bead quality. Variability between beads are examined in the following ways: size, sequence identity, sequence length, percentage of functionalization of the beads.

In addition, novel quality control methods have been developed, and quality control metrics will be generated at each step of the bead synthesis before proceeding with subsequent steps, which include: surface activation; spacer addition; phosphoramidite synthesis; and/or transcript capture efficiency.

Citation or identification of any document in this application is not an admission that such document is available as prior art to the present invention.

### SUMMARY

In one aspect, the present invention provides a composition comprising a solid support or plurality of solid supports. Each solid support may comprise one or more agents and optionally a spacer. In some embodiments, the solid support or plurality of solid supports comprises one or more beads or micro-bead or a plurality of micro-beads, micro-arrays, micro-wells, or micro-lids. The solid support may comprise a bead that is a silica bead, a hydrogel bead or a magnetic bead. The bead may have a shape that is circular, square, star, or it may be porous. In some embodiments, the solid support may comprise a magnetic core.

The solid support may have an average particle size between about 10 microns to 200 microns, about 10 microns to 30 microns, about 30 microns to 50 microns, about 50 microns to100 microns, about 100 microns to 200 microns, or about 30 microns. The bead or micro-bead may have an average size, measured as average diameter of 20-40 μm.

In some embodiments, the solid support comprises a polymer, optionally a hydroxylated methacrylic polymer, a hydroxylated poly(methyl methacrylate), a polystyrene polymer, a polypropylene polymer, a polyethylene polymer agarose, or cellulose. The spacer may comprise a polyethylene glycol polymer (PEG), a polysaccharide, an alkyl amine or a linker. In some embodiments, the spacer may comprise a PEG. The PEG may be a hetero-functional PEG comprising two or more different functionalities, wherein at least one of the functionalities is a primary amine or a thiol.

In some embodiments, the thiol may further comprise an acrydite moiety attached thereto. In some embodiments, the spacer may comprise a polyethylene glycol polymer (PEG) having a molecular weight range of about 1,000 daltons to 8,000 daltons. The PEG may have a molecular weight of about 1000 daltons, 2000 daltons, 3500 daltons, 5000 daltons, or 8000 daltons.

In some embodiments, the spacer may comprise a photolabile linker, a fluoride ion labile linker, or a cleavable linker. In some embodiments, the spacer may comprise a benzenesulfonylethyl linker, an o-nitrobenzyl carbonate photolabile linker, a 5-methoxy-2-nitrobenzyl carbonate photolabile linker, an o-nitrophenyl-1,3-propanediol base photolabile linker, a fluoride ion labile diisopropylsilyl linker, a fluoride ion labile disiloxyl phosphoramidite linker, a NPE carbonate linker, a 9-fluorenylmethyl linker, a phthaloyl linker, an oxalyl linker, a malonic acid linker, a succinyl linker, a diglycolic acid linker, a hydroquinone-O,O′-diacetic acid (Q-linker), or a thiophospate linker. In specific embodiments, the spacer comprises a benzenesulfonylethyl linker cleavable with triethylamine/dioxane.

In some embodiments, the spacer may comprise a nonyl phenol ethoxylate (NPE) carbonate linker cleavable with 1,8-Diazabicyclo[5.4.0]undec-7-ene (DBU)/pyridine. In some embodiments, the spacer may comprise a 9-fluorenylmethyl linker or a phthaloyl linker cleavable with DBU. In some embodiments, the spacer may comprise a succinic acid linked to an N-methylglycine (sarcosine) derivatized support, a succinic acid linked to 1,6-bis methylaminohexane spacer, a succinic acid linked to N-propyl polyethylene glycol Tentagel support, or a succinyl-sarcosine linkage.

The spacer may be grafted onto the solid support via an amine linkage, a secondary amine linkage, a thioether linkage, an ether linkage, a carbamate linkage, or an amide linkage.

The one or more agents may comprise one or more of oligonucleotides, nucleotides, analogs thereof; a molecular barcode; a Unique Molecular Identifier; an oligodT; an amplification primer; a cell type specific sequence; a pathogen-specific sequence, or a TCR specific sequence, and surface reactive nucleic acid molecule(s).

In some embodiments, the one or more agents the agents may comprise nucleic acid sequences of an in-silico Polymerase Chain Reaction (ISPCR) Primer, a Barcode, a Unique Molecular Identifier (UMI) and a Universal Sequence.

In some embodiments, the one or more agents are nucleic acid molecules in a 3′ to 5′ orientation. In alternative embodiments, the one or more agents are nucleic acid molecules in a 5′ to 3′ orientation.

In another aspect, the present invention provides a kit comprising a solid support having a surface bearing reacting groups; one or more activator(s) selected from 2-fluoro-1-methylpyridinium (FMP), carbonyl diimidazole (CDI) and a tosyl compound (Ts); a spacer compound; and optionally one or more nucleic acid molecules. In another aspect, the kit comprises a functionalized solid support having a spacer and one or more nucleic acid molecule(s).

In another aspect, the present invention provides a method for functionalizing a surface of a solid support, comprising: a) reacting a solid support having surface bearing reacting groups with an activator, so as to obtain a solid support with an activated surface comprising an activating moiety, b) reacting the activated surface with a spacer compound having a first moiety that reacts with the activating moiety and optionally a second moiety comprising a functional group, whereby the reacting of this step b) obtains, on a solid support, a spacer-grafted thereon, whereby the surface of the solid support is functionalized. In some aspects, the reacting of step b) obtains, on the solid support, a spacer-grafted thereon having the second moiety comprising the functional group exposed for reaction.

In some embodiments, the step a) reacting is under dry conditions or non-aqueous conditions or solid phase synthesis conditions.

The solid support may comprise a hydrogel bead or a magnetic bead. In some embodiments, the solid support may be a silica bead. The bead may have a shape that is circular, square, star, or it may be porous.

In some embodiments, the activator may comprise 2-Fluoro-1-Methylpyridinium (FMP), Carbonyl Diimidazole (CDI), bis-epoxide, divinylsulfone, cyanogen bromide, or an organic sulfonyl halide, optionally the organic sulfonyl halide comprises tosyl chloride or tresyl chloride. In some embodiments, the activating moiety comprises a tosyl group, imidazolyl carbamate group, or methylpyridinium group.

In some embodiments, the reacting groups on the surface of the solid support comprise a hydroxyl, a carboxyl, a thiol, an amine, a diol, or a combination thereof. The solid support may comprise a polymer. The polymer may be hydroxylated methacrylic polymer or hydroxylated poly(methyl methacrylate), polystyrene polymer, polypropylene polymer, polyethylene polymer, agarose, or cellulose.

The solid support may have an average particle size ranging between about 10 microns to 200 microns, about 10 microns to 30 microns, about 30 microns to 50 microns, about 50 microns to100 microns, about 100 microns to 200 microns, or about 30 microns.

In some embodiments, the spacer may comprise a polyethylene glycol polymer (PEG), a polysaccharide, an alkyl amine, or a succinyl linker. The spacer may comprise a photolabile linker, a fluoride ion labile linker, or a cleavable linker. The spacer may comprise a benzenesulfonylethyl linker, an o-nitrobenzyl carbonate photolabile linker, a 5-methoxy-2-nitrobenzyl carbonate photolabile linker, an o-nitrophenyl-1,3-propanediol base photolabile linker, a fluoride ion labile diisopropylsilyl linker, a fluoride ion labile disiloxyl phosphoramidite linker, a NPE carbonate linker, a 9-fluorenylmethyl linker, a phthaloyl linker, an oxalyl linker, a malonic acid linker, a diglycolic acid linker, a hydroquinone-O,O′-diacetic acid (Q-linker), or a thiophospate linker.

In some embodiments, the spacer may comprise a benzenesulfonylethyl linker cleavable with triethylamine/dioxane, a nonyl phenol ethoxylate (NPE) carbonate linker cleavable with 1,8-Diazabicyclo[5.4.0]undec-7-ene (DBU)/pyridine, or a 9-fluorenylmethyl linker or a phthaloyl linker cleavable with DBU. The spacer may comprise a succinic acid linked to an N-methylglycine (sarcosine) derivatized support, a succinic acid linked to 1,6-bis methylaminohexane spacer, a succinic acid linked to N-propyl polyethylene glycol Tentagel support, or a succinyl-sarcosine linkage. The spacer may comprise a polyethylene glycol polymer (PEG) having a molecular weight range of about 1,000 daltons to 8,000 daltons, about 1000 daltons, 2000 daltons, 3500 daltons, 5000 daltons, or 8000 daltons.

In some embodiments, the functional group exposed for reaction comprises a thiol group, a disulfide linkage, a hydroxyl group, or a phenyl group.

In some embodiments, the first moiety comprises an amine, an amide, a thiol, a carboxyl, or a hydroxyl group. In some embodiments, the first moiety comprises NH₂, the second moiety comprises SH, OH or phenyl, and the spacer comprises a PEG; or the first moiety-spacer-second moiety comprises: X—(Y)ₙ—Z, wherein X is a thiol, a hydroxyl, an amine, or a carboxyl, Y is PEG or a methylene group, and Z is a thiol, a hydroxyl, an amine, or a carboxyl, and wherein n is an integer between 1 and 30.

In some embodiments, the spacer is grafted onto the solid support via an amine linkage, a secondary amine linkage, a thioether linkage, an ether linkage, a disulfide linkage, or an amide linkage.

In another aspect, the present invention provides a method for preparing a population of functionalized solid support comprising a surface reactive nucleic acid molecule comprising a′) reacting a functionalized solid support having a spacer having a functional group exposed for reaction with a nucleic acid molecule so as to obtain a solid support comprising a surface reactive nucleic acid molecule.

In another aspect, the present invention provides a method for preparing a population of functionalized solid support comprising surface reactive nucleic acid molecules in sequence(s) comprising a″) reacting a solid support comprising a surface reactive nucleic acid molecule with another nucleic acid molecule so as to obtain a solid support comprising surface reactive nucleic acid molecules in sequence(s).

In some embodiments, the functionalized support having a spacer having a functional group exposed for reaction may be prepared by a method comprising a) reacting a solid support having surface bearing reacting groups with an activator, so as to obtain a solid support with an activated surface comprising an activating moiety, b) reacting the activated surface with a spacer compound having a first moiety that reacts with the activating moiety and optionally a second moiety comprising a functional group whereby the reacting of this step b) obtains, on the solid support, a spacer-grafted thereon, whereby the surface of the solid support is functionalized.

In some embodiments, step a″) may be repeated n times, wherein n is an integer between 1 and 100.

In some embodiments, step a″) is repeated so as to obtain surface reactive nucleic acid molecules in sequences of an ISPCR Primer, a Barcode, a Unique Molecular Identifier (UMI) and a Universal Sequence; or so that the surface reactive nucleic acid molecules comprise one or more of the following: oligonucleotides, nucleotides, analogs thereof, a molecular barcode, a Unique Molecular Identifier, a oligodT, an amplification primer, a cell type specific sequence, a pathogen-specific sequence, or a TCR specific sequence.

The nucleic acid molecules may be in a 3′ to 5′ orientation, or in a 5′ to 3′ orientation.

The solid support may comprise a bead or a plurality of beads, or a micro-bead or a plurality of micro-beads, micro-arrays, micro-wells, or micro-lids. The bead or micro-bead may have an average size, measured as average diameter of 20-40 μm. The solid support may comprise a magnetic core.

In some embodiments, the method for preparing a population of functionalized solid support is performed as to a plurality of solid supports.

Some embodiments may further comprise reacting the spacer with an acrydite, whereby the acrydite has the functional group exposed for reaction.

In another aspect, the present invention provides a solid support or a population or solid supports or one or more beads or micro-bead or a population of micro-beads, micro-arrays, micro-wells, or micro-lids prepared by any of the methods described herein.

In another aspect, the present invention provides a method for nucleic acid analysis wherein the method comprises single cell analysis or RNA analysis, DNA analysis, chromatin analysis or RNA-SEQ, or ATAC PCR. The method may also comprise processing an analyte comprising a protein, a peptide, an antibody, an organelle, a cell, a cellular fraction. The method may also be for processing a clinical sample, or may comprise single cell microfluidics analysis or DROP-SEQ. In some embodiments, the method may comprise a single cell microwell array analysis or SEQ-WELL. The method may also comprise a single cell microwell platform analysis, wherein the method comprises use of a solid support or plurality of solid supports as described herein, or solid support or plurality of solid supports or one or more beads or micro-bead or a plurality of micro-beads, micro-arrays, micro-wells, or micro-lids as described herein.

In some embodiments, the reacting of step b) obtains, on the solid support, a spacer-grafted thereon having the functional group exposed for reaction, whereby the surface of the solid support is functionalized. In one embodiment, the method provides a population of solid support that is at least 70% usable, i.e., at least 70% of beads produced by the method are functionalized for a particular use. In one embodiment, the method provides a population of solid support that is at least 80% usable. In another embodiment, the method provides a population of solid support that is at least 90%, at least 95%, or at least 98% usable.

In an embodiment of any of the above method, the first moiety comprises an amine, an amide, a thiol, a carboxyl, or a hydroxyl group. In an aspect of the embodiment, the first moiety comprises NH₂, the second moiety comprises SH, OH or phenyl, and the spacer comprises a PEG; or the first moiety-spacer-second moiety comprises: X—(Y)ₙ—Z, wherein X is a thiol, a hydroxyl, an amine, or a carboxyl, Y is PEG or a methylene group, and Z is a thiol, a hydroxyl, an amine, or a carboxyl. In another aspect of the embodiment, n is any integer from 1 to 170.

The present invention provides a method for characterizing a functionalized solid support having a spacer and a nucleic acid molecule, wherein the method comprises cleaving a disulfide linkage located between the spacer and the nucleic acid molecule, whereby the nucleic acid molecule is detached from the solid support. In one embodiment, the method comprises isolating the nucleic acid molecule. In another embodiment, the method comprises determining the molecular weight or mass of the nucleic acid molecule, e.g., via mass spectroscopy, chromatography, etc., or determining the sequence of the nucleic acid molecule using any known nucleic acid sequencing method, e.g., via PCR. In one embodiment, the method is used to validate a method for functionalizing the solid support. In some aspects, HPLC is used in the validation an quality control method.

The present invention provides a method for characterizing a functionalized solid support having a spacer, wherein the method comprises cleaving a linkage located between the spacer and the solid support, whereby the spacer is detached from the solid support. In some aspects, the linkage comprises a disulfide linkage, a photolabile linker, a halide ion labile linker, or any cleavable linker, the examples of which are described for the spacer herein. In one embodiment, the method comprises isolating the spacer. In another embodiment, the method comprises determining the mass or molecular weight of the spacer, e.g., via mass spectroscopy, chromatography, etc. In one embodiment, the method is used to validate a method for functionalizing the solid support.

In some aspects, the reacting in step a″) comprises removing a protecting group, e.g., DMT, from the 3′-terminal hydroxyl group of the surface reactive nucleotide. This is also known as the detritylation step. In some aspects, the reacting step further comprises coupling an nucleoside phosphoramidite to surface reactive nucleic acid and creating a phosphite triester. The unreacted 3′-hydroxyl groups are than capped with a protecting group, e.g., acetic anhydride and N-methylimidazole. The phosphate triester is then oxidized. In some aspects of the invention, step a″) is repeated n times, wherein n is 1 to 100. In some aspects, step a″ is repeated so as to obtain surface reactive nucleic acid molecules in sequences of an ISPCR Primer, a Barcode, a Unique Molecular Identifier (UMI) and/or a Universal Sequence; or so that the surface reactive nucleic acid molecules comprise one or more of the following: oligonucleotides, nucleotides, analogs thereof; a molecular barcode; a Unique Molecular Identifier; a oligodT; an amplification primer; a cell type specific sequence; a pathogen-specific sequence; or a TCR specific sequence. In some aspects, the nucleic acid molecules are added in a 5′ to 3′ fashion. Any commercially available amidites may be used for the reaction. For example, amidites may be purchased from http://www.glenresearch.com.

The invention provides for a method for nucleic acid analysis using the solid support described herein. The method for nucleic acid analysis can comprise single cell analysis, RNA analysis, DNA analysis, chromatin analysis or RNA-Seq. The method can comprise processing an analyte comprising a protein, a peptide, an antibody, an organelle, a cell, a cellular fraction, or processing a clinical sample. The method can comprise single cell microfluidics analysis or Drop-Seq (see WO2016/040476) or single cell microwell analysis (see WO/2017124101 and Jinzhou Yuan & Peter A. Sims, An Automated Microwell Platform for Large-Scale Single Cell RNA-Seq, Scientific Reports 6, 33883 (2016), doi:10.1038/srep33883).

In an embodiment of any of the above methods, the solid support comprises a bead or a population of beads, or a micro-bead or a population of micro-beads, micro-arrays, micro-wells, or micro-lids. In some aspect, the solid support comprises a hydrogel bead or a magnetic bead or a magnetic core. In an aspect of the embodiment, the solid support is a silica bead, a cellulose bead, or an agarose bead. In another aspect of the embodiment, the bead has a shape that is circular, square, star, or porous.

In an embodiment of any of the above method, the solid support comprises a polymer. In an aspect of the embodiment, the solid support comprises hydroxylated methacrylic polymer or hydroxylated poly(methyl methacrylate). In another aspect of the embodiment, the solid support comprises polystyrene polymer, polypropylene polymer, polyethylene polymer, agarose, or cellulose.

The present invention provides a composition comprising a population of functionalized solid support having a spacer and optionally having one or more nucleic acid molecules. In an embodiment, the population of functionalized solid support is prepared by a method for preparing described herein.

In an embodiment of any of the above method or composition, the solid support has an average particle size ranging from 10 microns to 500 microns.

In an embodiment of any of the above method of composition the solid support has an average particle size of about 10 to 30 microns.

In an embodiment of any of the above method of composition, the solid support has an average particle size of about 30 microns.

In an embodiment of any of the above method of composition, the solid support has an average particle size of about 30 to 50 microns.

In an embodiment of any of the above method or composition, the solid support has an average particle size of about 50-100 microns.

In an embodiment of any of the above method of composition, the solid support has an average particle size of about 100-200 microns.

In an embodiment of any of the above method of composition, the solid support has an average particle size of about 200-500 microns.

In an embodiment of any of the above method or composition, the spacer comprises a polyethylene glycol polymer (PEG), a polysaccharide, an alkyl amine or long chain alkyl amine, or a succinyl linker.

In an embodiment of any of the above method or composition, the spacer comprises a photolabile linker, a halide ion labile linker, or a cleavable linker. In some aspects, the spacer comprises a fluoride ion labile linker.

In an embodiment of any of the above method or composition, the spacer comprises an o-nitrobenzyl carbonate photolabile linker, a 5-methoxy-2-nitrobenzyl carbonate photolabile linker, an o-nitrophenyl-1,3-propanediol base photolabile linker, a fluoride ion labile diisopropylsilyl linker, a fluoride ion labile disiloxyl phosphoramidite linker, a NPE carbonate linker, a 9-fluorenylmethyl linker, a phthaloyl linker, an oxalyl linker, a malonic acid linker, a diglycolic acid linker, a hydroquinone-O,O′-diacetic acid (Q-linker), a thiophospate linker, or a disulfide linkage. In an aspect of the embodiment, the spacer comprises a benzenesulfonylethyl linker cleavable with triethylamine/dioxane. In an aspect of the embodiment, the spacer comprises a NPE carbonate linker cleavable with DBU/pyridine. In an aspect of the embodiment, the spacer comprises a 9-fluorenylmethyl linker or a phthaloyl linker cleavable with DBU. In another aspect of the embodiment, the spacer comprises a disulfide linkage cleavable with a reductant. Examples of a reductant includes β-mercaptomethanol (β-ME) and dithiothreitol (DTT).

In an embodiment of any of the above method or composition, the spacer comprises a succinic acid linked to an N-methylglycine (sarcosine) derivatized support, a succinic acid linked to 1,6-bis methylaminohexane spacer, a succinic acid linked to N-propyl polyethylene glycol Tentagel support, or a succinyl-sarcosine linkage.

In an embodiment of any of the above method or composition, the spacer comprises a polyethylene glycol polymer (PEG) having an average molecular weight range of about 500 daltons to 8,000 daltons. In some aspects, the spacer comprises a PEG having about 1 to 12 monomer repeats. In an aspect of the embodiment, the spacer comprises PEG polymer having an average molecular weight of about 1000 daltons, about 2000 daltons, about 3500 daltons, about 4000 daltons, about 5000 daltons, about 7500 daltons, or about 8000 daltons.

In an embodiment of any of the above method or composition, the spacer is grafted onto the solid support via an amine linkage, a secondary amine linkage, a thioether linkage, an ether linkage, a disulfide linkage, or an amide linkage.

These and other aspects, objects, features, and advantages of the example embodiments will become apparent to those having ordinary skill in the art upon consideration of the following detailed description of illustrated example embodiments.

### BRIEF DESCRIPTION OF THE DRAWINGS

An understanding of the features and advantages of the present invention will be obtained by reference to the following detailed description that sets forth illustrative embodiments, in which the principles of the invention may be utilized, and the accompanying drawings of which:

FIG. 1 demonstrates an exemplary method of activation of a solid support with carbonyl diimidazole (CDI).

FIG. 2 demonstrates an exemplary method of activation of a solid support with 2-fluoro-1-methylpyridinium (FMP).

FIG. 3 demonstrates an exemplary method of activation of a solid support with tosyl chloride.

FIG. 4A-4C show exemplary bead synthesis diagrams, FIG. 4A provides a solid support with spacer and acrydite linker; FIG. 4B depicts a solid support with no spacer; FIG. 4C depicts a solid support with PEG spacer and no linker. In the bead synthesis diagrams, cleavable linkers may be included between the bead and the first appended molecule, or between other molecules, e.g. between a spacer and an oligonucleotide, depending on desired end use.

FIG. 5A-5M: Examples of linkers: FIG. 5A o-Nitrobenzyl carbonate photolabile linker arm (Greenberg and Gilmore, 1994). FIG. 5B 5-Methoxy-2-nitrobenzyl carbonate photolabile linker arms (Venkatesan and Greenberg, 1996). FIG. 5C o-Nitrophenyl-1,3-propanediol base photolabile linker for 3′-phosphorylated oligonu-cleotides (Dell'Aquila et al., 1997). FIG. 5D Fluoride ion labile diisopropylsilyl linker arm (Routledge et al., 1995). FIG. 5E Fluoride ion labile disiloxyl phosphoramidite linker arm (Kwiatkowski et al., 1996). FIG. 5F Benzenesulfonylethyl linker arm cleavable with triethylamine/dioxane (Efimov et al., 1983). FIG. 5G NPE carbonate linker arm cleavable with DBU/pyridine (Eritja et al., 1991). FIG. 5I1 9-Fluorenylmethyl linker cleavable with DBU (Avino et al., 1996). FIG. 5I Phthaloyl linker arm cleavable with DBU (Avino et al., 1996; Brown et al., 1989). FIG. 5J Oxalyl linker, cleavable under very mild conditions (Alul et al., 1991). FIG. 5K Malonic acid linker for the synthesis of 3′-phosphorylated oligonucleotides (Guzaev and Lonnberg, 1997). FIG. 5L Diglycolic acid linker used to make 3′-TAMRA dye-labeled oligonucleotides (Mullah et al., 1998). FIG. 5M Hydroquinone-O,O′-diacetic acid (Q-linker), which can be used for routine oligonucleotides to improve synthesis productivity or to synthesize base-labile products (Pon and Yu, 1997a).

FIG. 6A-6J: Examples of linker arms that allow on-column deprotectation and then optional cleavage: FIG. 6A Succinic acid linked to an N-methylglycine (sarcosine) derivatized support (Brown et al., 1989). FIG. 6B Succinic acid linked to 1,6-bis methylaminohexane spacer (Stengele and Pfleiderer, 1990). FIG. 6C Succinic acid linked to N-propyl polyethylene glycol Tentagel support (Weiler and Pfleiderer, 1995). FIG. 6D Succinyl-sarcosine linkage for the solid-phase synthesis of branched oligonucleotides (Grotli et al., 1997). FIG. 6E Linkage through the amino group of cytosine for branched and cyclic oligonu-cleotide synthesis (De Napoli et al., 1995). FIG. 6F Oxidizable solid support (Bower et al., 1987; Markiewicz et al., 1994). FIG. 6G Phenyl thioether linker, which is stable until oxidized into a phenylsul-fone (Felder et al., 1984). FIG. 6H Thiophosphate linker, cleavable by iodine/water oxidation or acetic acid hydrolysis (Tanaka et al., 1989). FIG. 6I 3-Chloro-4-hydroxyphenyl linker for the solid-phase synthesis of cyclic oligonucleotides (Alazzouzi et al., 1997). FIG. 6J Linker arm produced from tolylene 2,6-diisocyanate with more stable carbamate and urethane linkages (Kumar, 1994; Sproat and Brown, 1985).

FIG. 7A-7L: Examples of linker arms for permanent attachment to solid-phase supports: FIG. 7A Hydroxy propylamine linker (Seliger et al., 1995). FIG. 7B Dimethoxytrityl glycolic acid linker (Hakala et al., 1997). FIG. 7C Dimethoxytrityl-4,7,10,13-tetraoxatridecanoate linker (Markiewicz et al., 1994). FIG. 7D Long spacer linkages prepared using repetitive coupling of various phosphoramidites (Shchepinov et al., 1997). FIG. 7E Cleavable spacer linkage used in conjunction with the preceding phosphoramidites to control the surface oligonucleotide density (Shchepinov et al., 1997), FIG. 7F Direct phosphate linkage to surface silanol groups (Cohen et al., 1997). FIG. 7G Diol linker formed from 3-glycidoxypropyl trimethoxysilane (Maskos and Southern, 1992). FIG. 7H Polyethylene glycol linkers (Maskos and Southern, 1992). FIG. 7I Bis-(2-hydroxethyl)-aminopropylsilane linker with hexaethylene glycol spacer phosphoramidites (Pease et al., 1994). FIG. 7J N-(3-(triethoxysilyl)-propyl)-4-hydroxybutyramide linker (McGall et al., 1997). FIG. 7K Linkage through the N⁴-position of cytosine (Markiewicz et al., 1994). FIG. 7L Triethylene glycol ethylacrylamide linker (Markiewicz et al., 1994).

FIG. 8: Exemplary oligo-synthesis strategy.

FIG. 9A-9C: Bead synthesis scheme. FIG. 9A demonstrates generating barcodes for capture beads. FIG. 9B shows extending barcodes and extending sequence by adding additional oligonucleotides. FIG. 9C shows an example of a capture bead with oligonucleotide sequence.

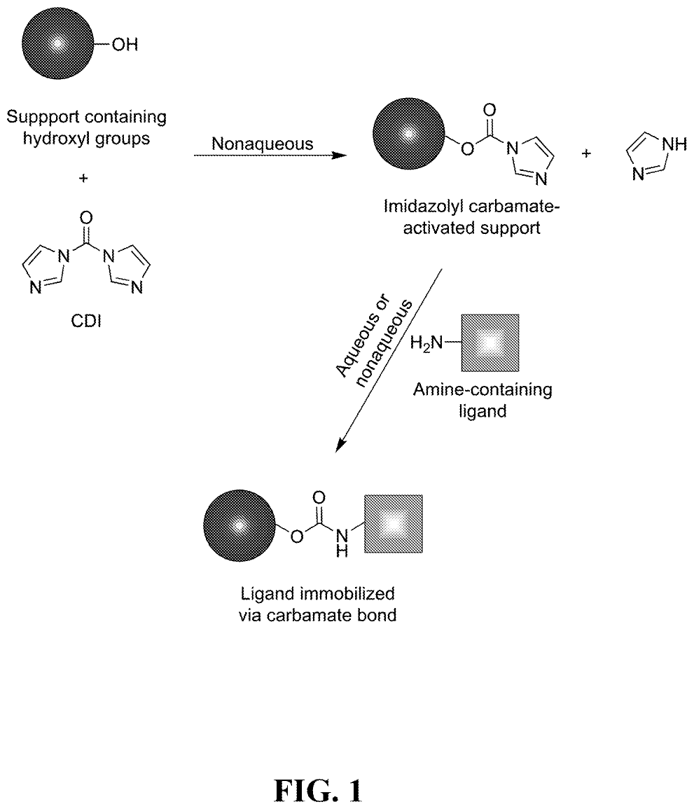

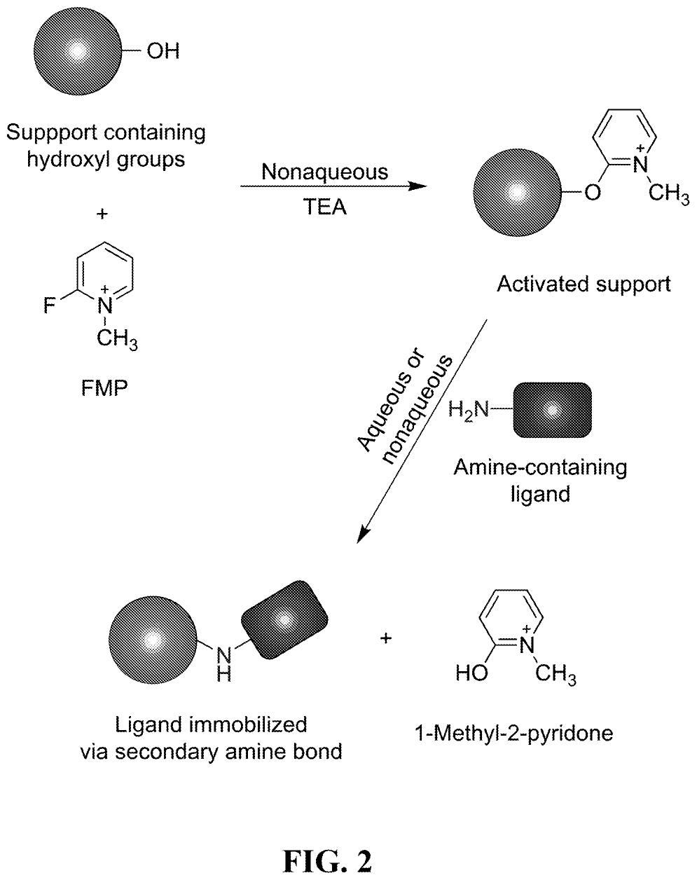

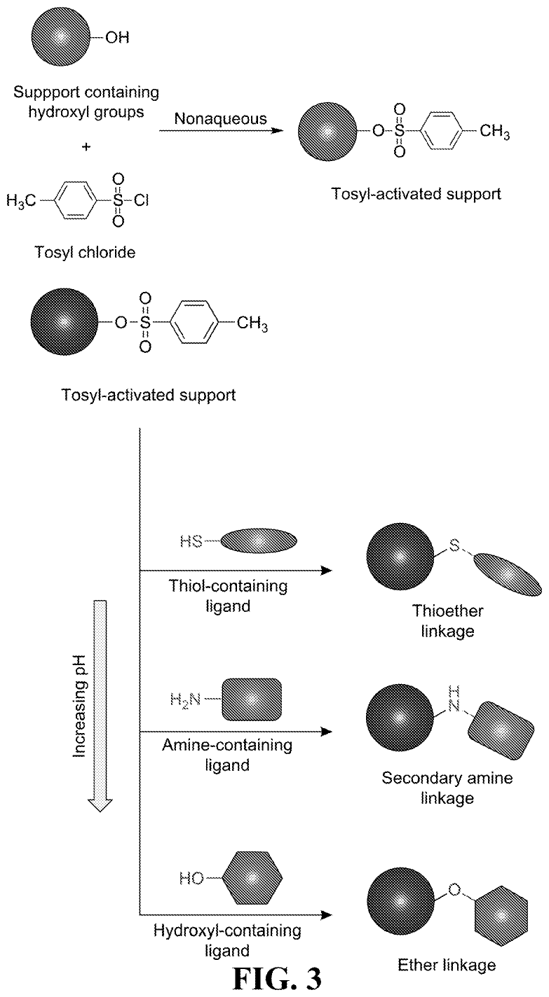

The figures herein are for illustrative purposes only and are not necessarily drawn to scale.

### DETAILED DESCRIPTION OF THE EXAMPLE EMBODIMENTS

### General Definitions

Unless defined otherwise, technical and scientific terms used herein have the same meaning as commonly understood by one of ordinary skill in the art to which this disclosure pertains. Definitions of common terms and techniques in molecular biology may be found in Molecular Cloning: A Laboratory Manual, 2^{nd} edition (1989) (Sambrook, Fritsch, and Maniatis); Molecular Cloning: A Laboratory Manual, 4^{th} edition (2012) (Green and Sambrook); Current Protocols in Molecular Biology (1987) (F. M. Ausubel et al. eds.); the series Methods in Enzymology (Academic Press, Inc.): PCR 2: A Practical Approach (1995) (M. J. MacPherson, B. D. Hames, and G. R. Taylor eds.): Antibodies, A Laboratory Manual (1988) (Harlow and Lane, eds.): Antibodies A Laboratory Manual, 2^{nd} edition 2013 (E. A. Greenfield ed.); Animal Cell Culture (1987) (R. I. Freshney, ed.); Benjamin Lewin, Genes IX, published by Jones and Bartlet, 2008 (ISBN 0763752223); Kendrew et al. (eds.), The Encyclopedia of Molecular Biology, published by Blackwell Science Ltd., 1994 (ISBN 0632021829); Robert A. Meyers (ed.), Molecular Biology and Biotechnology: a Comprehensive Desk Reference, published by VCH Publishers, Inc., 1995 (ISBN 9780471185710); Singleton et al., Dictionary of Microbiology and Molecular Biology 2nd ed., J. Wiley & Sons (New York, N.Y. 1994), March, Advanced Organic Chemistry Reactions, Mechanisms and Structure 4th ed., John Wiley & Sons (New York, N.Y. 1992); and Marten H. Hofker and Jan van Deursen, Transgenic Mouse Methods and Protocols, 2^{nd} edition (2011) .

As used herein, the singular forms “a”, “an”, and “the” include both singular and plural referents unless the context clearly dictates otherwise.

The term “optional” or “optionally” means that the subsequent described event, circumstance or substituent may or may not occur, and that the description includes instances where the event or circumstance occurs and instances where it does not.

The recitation of numerical ranges by endpoints includes all numbers and fractions subsumed within the respective ranges, as well as the recited endpoints.

The terms “about” or “approximately” as used herein when referring to a measurable value such as a parameter, an amount, a temporal duration, and the like, are meant to encompass variations of and from the specified value, such as variations of +/−10% or less, +/−5% or less, +/−1% or less, and +/−0.1% or less of and from the specified value, insofar such variations are appropriate to perform in the disclosed invention. It is to be understood that the value to which the modifier “about” or “approximately” refers is itself also specifically, and preferably, disclosed.

As used herein, a “biological sample” may contain whole cells and/or live cells and/or cell debris. The biological sample may contain (or be derived from) a “bodily fluid”. The present invention encompasses embodiments wherein the bodily fluid is selected from amniotic fluid, aqueous humour, vitreous humour, bile, blood serum, breast milk, cerebrospinal fluid, cerumen (earwax), chyle, chyme, endolymph, perilymph, exudates, feces, female ejaculate, gastric acid, gastric juice, lymph, mucus (including nasal drainage and phlegm), pericardial fluid, peritoneal fluid, pleural fluid, pus, rheum, saliva, sebum (skin oil), semen, sputum, synovial fluid, sweat, tears, urine, vaginal secretion, vomit and mixtures of one or more thereof. Biological samples include cell cultures, bodily fluids, cell cultures from bodily fluids. Bodily fluids may be obtained from a mammal organism, for example by puncture, or other collecting or sampling procedures.

The terms “subject,” “individual,” and “patient” are used interchangeably herein to refer to a vertebrate, preferably a mammal, more preferably a human. Mammals include, but are not limited to, murines, simians, humans, farm animals, sport animals, and pets. Tissues, cells and their progeny of a biological entity obtained in vivo or cultured in vitro are also encompassed.

Various embodiments are described hereinafter. It should be noted that the specific embodiments are not intended as an exhaustive description or as a limitation to the broader aspects discussed herein. One aspect described in conjunction with a particular embodiment is not necessarily limited to that embodiment and can be practiced with any other embodiment(s). Reference throughout this specification to “one embodiment”, “an embodiment,” “an example embodiment,” means that a particular feature, structure or characteristic described in connection with the embodiment is included in at least one embodiment of the present invention. Thus, appearances of the phrases “in one embodiment,” “in an embodiment,” or “an example embodiment” in various places throughout this specification are not necessarily all referring to the same embodiment, but may. Furthermore, the particular features, structures or characteristics may be combined in any suitable manner, as would be apparent to a person skilled in the art from this disclosure, in one or more embodiments. Furthermore, while some embodiments described herein include some but not other features included in other embodiments, combinations of features of different embodiments are meant to be within the scope of the invention. For example, in the appended claims, any of the claimed embodiments can be used in any combination.

Reference is made to U.S. Provisional Application No. 62/575,883, filed Oct. 23, 2017 and PCT/US2013/060990 filed Mar. 13, 201, incorporated by reference in their entirety.

All publications, published patent documents, and patent applications cited herein are hereby incorporated by reference to the same extent as though each individual publication, published patent document, or patent application was specifically and individually indicated as being incorporated by reference.

### Overview

Embodiments disclosed herein provide a functionalized solid support or a population of functionalized solid support having a spacer. The solid support described herein may be used in generating libraries of unique labels or barcodes. Such libraries may be of any size, and are preferably large libraries including hundreds of thousands to billions of unique labels.

Methods of making the functionalized solid supports disclosed herein are also provided. The methods developed by Applicants increase the quality of solid supports, for example, by allowing for a higher percentage of functionalization, sn increase in the number of reactive sites, higher oligo density, higher transcript capture efficiency, an increase in oligo sequence consistency in terms of identity and length of sequences, and monodisperity, i.e., particle size uniformity.

Some methods of the invention comprise determining information about an agent based on the unique label associated with the agent. In some instances, determining information about the agent may comprise obtaining the nucleotide sequence of the unique label (i.e., sequencing the unique label). In other instances, determining information about the agent may comprise determining the presence, number and/or order of non-nucleic acid detectable moieties. In still other instances, determining information about the agent may comprise obtaining the nucleotide sequence of the unique label and determining the presence, number and/or order of non-nucleic acid detectable moieties. Methods for nucleic acid sequencing and detection of non-nucleic acid detectable moieties are known in the art and are described herein.

### Functionalized Solid Support

In an embodiment, the present invention provides a solid support that has been functionalized to comprise one or more agents.

A solid support can mean a bead or micro-bead, or a plurality of micro-beads, micro-arrays, micro-wells, or micro-lids. The solid support can be shaped in any manner required for an end use application and may have a shape that is circular, square, star, or porous. Examples of suitable solid supports include, but are not limited to, inert polymers (preferably non-nucleic acid polymers), beads, glass, or peptides. In some embodiments, the solid support is an inert polymer or a bead. The bead is a silica bead, a hydrogel bead or a magnetic bead. In some embodiments, the solid support comprises a magnetic core.

Examples of suitable polymers include a hydroxylated methacrylic polymer, a hydroxylated poly(methyl methacrylate), a polystyrene polymer, a polypropylene polymer, a polyethylene polymer agarose, or cellulose.

The solid support may be functionalized to permit covalent attachment of the agent and/or label. Such functionalization on the support may comprise reactive groups that permit covalent attachment to an agent and/or a label.

In particular embodiments, the solid support has an average particle size between about 10 microns to 200 microns, about 10 microns to 190 microns, about 10 microns to 180 microns, about 10 microns to 170 microns, about 10 microns to 160 microns, about 10 microns to 150 microns, about 10 microns to about 140 microns, about 10 to about 130 microns, about 10 to about 120 microns, about 10 microns to about 110 microns, about 10 microns to about 100 microns, about 10 microns to about 90 microns, about 10 microns to about 80 microns, about 10 microns to about 70 microns, about 10 microns to about 60 microns, about 10 microns to about 50 microns, about 10 microns to about 40 microns, about 10 microns to 30 microns, about 10 microns to about 20 microns, about 20 microns to about 30 microns, about 20 microns to about 40 microns, about 20 microns to about 50 microns, about 20 microns to about 60 microns, about 20 microns to about 70 microns, about 20 microns to about 80 microns, about 20 microns to about 100 microns, about 20 microns to about 100 microns, about 50 microns to about 100 microns, about 100 microns to 200 microns, or about 30 microns. In some embodiments, the bead or micro-bead has an average size, measured as average diameter of 20-40 μm.

### Spacers

A spacer as used herein, may comprise a polyethylene glycol polymer (PEG), a polysaccharide, an alkyl amine or a linker. Spacers can be optionally included in the solid supports. In some embodiments, the spacer further comprises a linker.

In particular embodiments, the spacer is preferably a PEG spacer or non-PEG spacer, e.g., long chain alkyl amines (LCAA) (see also Guzaev, Andrei, “Solid-Phase Supports for Oligonucleotide Synthesis,” Current Protocols in Nucleic Acid Chemistry (2010) 3.1.1-3.1.28), and the spacer can be a linker or comprise a linker.

In addition, a spacer can include linker arms that allow on-column deprotection and then optional cleave. For example, succinic acid linked to an N-methylglycine (sarcosine) derivatized support (Brown, T., Pritchard, C. E., Turner, G., and Salisbury, S. A. 1989. A new base-stable linker for solid-phase oligonucleotide synthesis. J. Chem. Soc. Chem. Commun. 891-893.), succinic acid linked to 1,6-bis methylaminohexane spacer (Stengele, K. P. and Pfleiderer, W. 1990. Improved synthesis of oligodeoxyribonucleotides. Tetra-hedron Lett. 31:2549-2552.), succinic acid linked to N-propyl polyethylene glycol Tentagel support (Weiler, J. and Pfleiderer, W. 1995. An improved method for the large-scale synthesis of oligonucleotides applying the NPE/NPEOC strategy. Nucleos. Nucleot. 14:917-920.), succinyl-sarcosine linkage for the solid-phase synthesis of branched oligonucleotides (Grotli, M., Eritja, R., and Sproat, B. 1997. Solid phase synthesis of branched RNA and branched DNA/RNA chimeras. Tetrahedron 53:11317-11347.), linkage through the amino group of cytosine for branched and cyclic oligonucleotide synthesis (De Napoli, L., Galeone, A., Mayol, L., Messere, A., Montesarchio, D., and Piccialli, G. 1995. Auto-mated solid phase synthesis of cyclic oligonu-cleotides: A further improvement. Bioorgan. Med. Chem. 3:1325-1329.), Oxidizable solid support (Bower, M., Summers, M. F., Kell, B., Hoskins, J., Zon, G., and Wilson, W. D. 1987. Synthesis and characterization of oligodeoxyribonucleotides containing terminal phosphates. NMR, UV spectroscopic and thermodynamic analysis of duplex formation of [d(pGGATTCC)]₂ (SEQ ID NO:1), [d(GGAAT-TCCp)]₂ (SEQ ID NO:2) and [d(pGGAATTCCp)]₂ (SEQ ID NO:3). Nucl. Acids Res. 15:3531-3547.; Markiewicz, W. T., Adrych-Rozek, K., Markiewicz, M., Zebrowska, A., and Astriab, A. 1994. Synthesis of oligonucleotides permanently linked with solid supports for use as synthetic oligonucleotide combinatorial libraries. In Innovation and Perspectives in Solid Phase Synthesis: Peptides, Proteins and Nucleic Acids: Biological and Biomedical Applications (R. Epton, ed.) pp. 339-346. Mayflower Worldwide, Birmingham.), Phenyl thioether linker, which is stable until oxidized into a phenylsulfone (Felder, E., Schwyzer, R., Charubala, R., Pfleiderer, W., and Schulz, B. 1984. A new solid phase approach for rapid synthesis of oligonucleotides bearing a 3′-terminal phosphate group. Tetrahedron Lett. 25:3967-3970.), Thiophosphate linker, cleavable by iodine/water oxidation or acetic acid hydrolysis (Tanaka, T., Yamada, Y., Uesugi, S., and Ikehara, M. 1989. Preparation of a new phosphorylating agent: S—(N-monomethoxytritylaminoethyl)-O-(o-chlorophenyl) phosphorothioate and its application in oligonucleotide synthesis. Tetrahedron 45:651-660.), 3-Chloro-4-hydroxyphenyl linker for the solid-phase synthesis of cyclic oligonucleotides (Alazzouzi, E., Escaja, N., Grandas, A., and Pedroso, E. 1997. A straightforward solid-phase synthesis of cyclic oligodeoxyribonucleotides. Angew. Chem. Intl. Ed. Engl. 36:1506-1508.), Linker arm produced from tolylene 2,6-diisocyanate with more stable carbamate and urethane linkages (Kumar, A. 1994. Development of a suitable linkage for oligonucleotide synthesis and preliminary hybridization studies on oligonucleotides synthesized in situ. Nucleos. Nucleot. 13:2125-2134.; Sproat, B. S. and Brown, D. M. 1985. A new linkage for solid phase synthesis of oligodeoxyribonucleotides. Nucl. Acids Res. 13:2979-2987.). A spacer can also include linker arms for permanent attachment to solid-phase supports, e.g., Hydroxy propylamine linker (Seliger, H., Bader, R., Birch-Hirschfield, E., Földes-Papp, Z., Hinz, M., and Scharpf, C. 1995. Surface reactive polymers for special applications in nucleic acid synthesis. Reactive Functional Polymers 26:119-126.), Dimethoxytrityl glycolic acid linker (Hakala, H., Heinonen, P., Iitia, A., and Lonnberg, H. 1997. Detection of oligonucleotide hybridization on a single microparticle by time-resolved fluorometry: Hybridization assays on polymer particles obtained by direct solid phase assembly of the oligonucleotide probes. Bioconjugate Chem. 8:378-384.), Dimethoxytrityl-4,7,10,13-tetraoxatridecanoate linker (Markiewicz, W. T., Adrych-Rozek, K., Markiewicz, M., Zebrowska, A., and Astriab, A. 1994. Synthesis of oligonucleotides permanently linked with solid supports for use as synthetic oligonucleotide combinatorial libraries. In Innovation and Perspectives in Solid Phase Synthesis: Peptides, Proteins and Nucleic Acids: Biological and Biomedical Applications (R. Epton, ed.) pp. 339-346. Mayflower Worldwide, Birmingham.), Long spacer linkages prepared using repetitive coupling of various phosphoramidites (Shchepinov, M. S., CaseGreen, S. C., and Southern, E. M. 1997. Steric factors influencing hybridisation of nucleic acids to oligonucleotide arrays. Nucl. Acids Res. 25:1155-1161.), Cleavable spacer linkage used in conjunction with the preceding phosphoramidites to control the surface oligonucleotide density (Shchepinov, M. S., CaseGreen, S. C., and Southern, E. M. 1997. Steric factors influencing hybridisation of nucleic acids to oligonucleotide arrays. Nucl. Acids Res. 25:1155-1161.), Direct phosphate linkage to surface silanol groups (Cohen, G., Deutsch, J., Fineberg, J., and Levine, A. 1997. Covalent attachment of DNA oligonucleotides to glass. Nucl. Acids Res. 25:911-912.), Diol linker formed from 3-glycidoxypropyl trimethoxysilane (Maskos, U. and Southern, E. M. 1992. Oligonucleotide hybridizations on glass supports: A novel linker for oligonucleotide synthesis and hybridisation properties of oligonucleotides synthesized in situ. Nucl. Acids Res. 20:1679-1684.), Polyethylene glycol linkers (Maskos, U. and Southern, E. M. 1992. Oligonucleotide hybridizations on glass supports: A novel linker for oligonucleotide synthesis and hybridisation properties of oligonucleotides synthesized in situ. Nucl. Acids Res. 20:1679-1684.), Bis-(2-hydroxethyl)-aminopropylsilane linker with hexaethylene glycol spacer phosphoramidites (Pease, A. C., Solas, D., Sullivan, E. J., Cronin, M. T., Holmes, C. P., and Fodor, S. P. A. 1994. Light-generated oligonucleotide arrays for rapid DNA sequence analysis. Proc. Natl. Acad. Sci. U.S.A. 91:5022-5026.), N-(3-(triethoxysilyl)-propyl)-4-hydroxybutyramide linker (McGall, G. H., Barone, A. D., Diggelmann, M., Fo-dor, S. P. A., Gentalen, E., and Ngo, N. 1997. The efficiency of light-directed synthesis of DNA arrays on glass substrates. J. Am. Chem. Soc. 119:5081-5090.), Linkage through the N⁴-position of cytosine (Markiewicz, W. T., Adrych-Rozek, K., Markiewicz, M., Zebrowska, A., and Astriab, A. 1994. Synthesis of oligonucleotides permanently linked with solid supports for use as synthetic oligonucleotide combinatorial libraries. In Innovation and Perspectives in Solid Phase Synthesis: Peptides, Proteins and Nucleic Acids: Biological and Biomedical Applications (R. Epton, ed.) pp. 339-346. Mayflower Worldwide, Birmingham.), Triethylene glycol ethylacrylamide linker (Markiewicz, W. T., Adrych-Rozek, K., Markiewicz, M., Zebrowska, A., and Astriab, A. 1994. Synthesis of oligonucleotides permanently linked with solid supports for use as synthetic oligonucleotide combinatorial libraries. In Innovation and Perspectives in Solid Phase Synthesis: Peptides, Proteins and Nucleic Acids: Biological and Biomedical Applications (R. Epton, ed.) pp. 339-346. Mayflower Worldwide, Birmingham.).

A spacer is preferably multifunctional, in some embodiments bifunctional, and can be either homo-functional or hetero-functional. For example, a PEG spacer can have an amine on one end and a thiol on the other end of the molecule, or amine and hydroxyl heterobufunctional PEGs. The functionality of the spacer may be capped with protecting groups. Examples of hydroxyl protecting groups include e.g., 3-nitro-2-pyridinesulfenyl (Npys); dimethoxytrityl (DMT); monomethyoxytrityl (MMT); Acetyl; benzyl; Benzoyl; Beta-methoxyethoxymethyl ether; Methoxymethyl ether; p-methyoxybenzyl ether; methyl-thiol methyl ether; Pivaloyl (Piv); Tetrahydropyranyl (THP); Tetrahydrofuran (THF); Trityl; Silyl ether; Methyl ether; and Ethoxy ethyl ethers. Examples of amine protecting groups include, e.g., Carbobenzyloxy; p-Methoxybenzylcarbonyl; tert-Butyloxycarbonyl; 9-fluorenylmethyloxycarbonyl (FMOC); Acetyl; Benzoyl; Benzyl; Carbamate; p-Methyoxybenzyl; 3,4-dimethoxybenzyl; p-methoxyphenyl; Tosyl; TROC; NOSL; and NPS. Examples of carboxylic acid protecting groups include, e.g., Methyl Esters; Benzyl Esters; Tert-butyl esters; Esters of 2,6 disubstituted phenyls; Silyl esters; Orthoesters; and Oxazoline. Examples of phosphate protecting groups include, e.g., 2-cyanoethyl and methyl. Examples of terminal alkyne protecting groups include, e.g., propargyl alcohols and silyl groups.

In another example, a label can be attached to an agent via a linker or in another indirect manner. Examples of linkers, include, but are not limited to, carbon-containing chains, polyethylene glycol (PEG), nucleic acids, monosaccharide units, and peptides. The linkers may be cleavable under certain conditions.

Cleavable linkers are known in the art and include, but are not limited to, TEV, trypsin, thrombin, cathepsin B, cathespin D, cathepsin K, caspase 1, matrix metalloproteinase sequences, phosphodiester, phospholipid, ester, β-galactose, dialkyl dialkoxysilane, cyanoethyl group, sulfone, ethylene glycolyl disuccinate, 2-N-acyl nitrobenzenesulfonamide, a-thiophenylester, unsaturated vinyl sulfide, sulfonamide after activation, malondialdehyde (MDA)-indole derivative, levulinoyl ester, hydrazone, acylhydrazone, alkyl thioester, disulfide bridges, azo compounds, 2-Nitrobenzyl derivatives, phenacyl ester, 8-quinolinyl benzenesulfonate, coumarin, phosphotriester, bis-arylhydrazone, bimane bi-thiopropionic acid derivative, paramethoxybenzyl derivative, tert-butylcarbamate analogue, dialkyl or diaryl dialkoxysilane, orthoester, acetal, aconityl, hydrazone, b-thiopropionate, phosphoramidate, imine, trityl, vinyl ether, polyketal, alkyl 2-(diphenylphosphino)benzoate derivatives, allyl ester, 8-hydroxyquinoline ester, picolinate ester, vicinal diols, and selenium compounds (see, e.g. Leriche G, Chisholm L, Wagner A. Cleavable Linkers in Chemical Biology. Bioorg Med Chem. 15; 20(2):571-82. 2012, which is incorporated herein by reference). Cleavage conditions and reagents include, but are not limited to, enzymes, nucleophilic/basic reagents, reducing agents, photo-irradiation, electrophilic/acidic reagents, organometallic and metal reagents, and oxidizing reagents.

Examples of linkers include a succinyl linker, o-Nitrobenzyl carbonate photolabile linker arm (Greenberg, M. M. and Gilmore, J. L. 1994. Cleavage of oligonucleotides from solid-phase supports using O-nitrobenzyl photochemistry. J. Org. Chem. 59:746-753.), 5-Methoxy-2-nitrobenzyl carbonate photolabile linker arms (Venkatesan, H. and Greenberg, M. M. 1996. Improved utility of photolabile solid phase synthe-sis supports for the synthesis of oligonucleotides containing 3′-hydroxyl termini. J. Org. Chem. 61:525-529.), o-Nitrophenyl-1,3-propanediol base photolabile linker for 3′-phosphorylated oligonucleotides (Dell' Aquila, C., Imbach, J. L., and Rayner, B. 1997. Photolabile linker for the solid-phase synthesis of base-sensitive oligonucleotides. Tetrahedron Lett. 38:5289-5292.), Fluoride ion labile diisopropylsilyl linker arm (Routledge, A., Wallis, M. P., Ross, K. C., and Fraser, W. 1995. A new deprotection strategy for automated oligonucleotide synthesis using a novel silyl-linked solid support. Bioorgan. Med. Chem. Lett. 5:2059-2064.), Fluoride ion labile disiloxyl phosphoramidite linker arm (Kwiatkowski, M., Nilsson, M., and Landegren, U. 1996. Synthesis of full-length oligonucleotides: Cleavage of apurinic molecules on a novel sup-port. Nucl. Acids Res. 24:4632-4638.), Benzenesulfonylethyl linker arm cleavable with triethylamine/dioxane (Efimov, V. A., Buryakova, A. A., Reverdatto, S. V., Chakhmakhcheva, O. G., and Ovchinnikov, Y. A. 1983. Rapid synthesis of long-chain deoxyribooligonucleotides by the N-methylimidazole phosphotriester method. Nucl. Acids Res. 11:8369-8387.), NPE carbonate linker arm cleavable with DBU/pyridine (Eritja, R., Robles, J., Fernandezforner, D., Albericio, F., Giralt, E., and Pedroso, E. 1991. NPE-resin, a new approach to the solid-phase synthesis of protected peptides and oligonucleotides. 1. Synthesis of the supports and their application to oligonucleotide synthesis. Tetrahedron Lett. 32:1511-1514.), 9-Fluorenylmethyl linker or phthaloyl linker arm cleavable with DBU (Avino, A., Garcia, R. G., Diaz, A., Albericio, F., and Eritja, R. 1996. A comparative study of supports for the synthesis of oligonucleotides without using ammonia. Nucleos. Nucleot. 15:1871-1889; Brown, T., Pritchard, C. E., Turner, G., and Salisbury, S. A. 1989. A new base-stable linker for solid-phase oligonucleotide synthesis. J. Chem. Soc. Chem. Commun. 891-893.), Oxalyl linker, cleavable under very mild conditions (Alul, R. H., Singman, C. N., Zhang, G. R., and Letsinger, R. L. 1991. Oxalyl-CPG—A labile support for synthesis of sensitive oligonucleotide derivatives. Nucl. Acids Res. 19:1527-1532.), Malonic acid linker for the synthesis of 3′-phosphorylated oligonucleotides (Guzaev, A. and Lonnberg, H. 1997. A novel solid support for synthesis of 3′-phosphorylated chimeric oligonucleotides containing internucleosidic methyl phosphotriester and methyl-phosphonate linkages. Tetrahedron Lett. 38:3989-3992.), Diglycolic acid linker used to make 3′-TAMRA dye-labeled oligonucleotides (Mullah, B., Livak, K., Andrus, A., and Kenney, P. 1998. Efficient synthesis of double dye-labeled oligodeoxyribonucleotide probes and their application in a real time PCR assay. Nucl. Acids Res. 26:1026-1031.), Hydroquinone-O,O′-diacetic acid (Q-linker), which can be used for routine oligonucleotides to improve synthesis productivity or to synthesize base-labile products (Pon, R. T. and Yu, S. 1997a. Hydroquinone-O,O′-diacetic acid (‘Q-linker’) as a replacement for succinyl and oxalyl linker arms in solid phase oligonucleotide synthesis. Nucl. Acids Res. 25:3629-3635.).

In some instances, spacer comprises a polyethylene glycol polymer (PEG) having a molecular weight range of about 300 daltons, about 400 daltons, about 500 daltons, about 600 daltons, about 700 daltons, about 800 daltons, about 900 daltons, about 1,000 daltons to 8,000 daltons. The PEG may have a molecular weight of about 300 daltons, about 500 daltons, about 800 daltons, about 1000 daltons, about 1500 daltons, about 2000 daltons, about 3500 daltons, about 5000 daltons, or about 8000 daltons. In some embodiments, the PEG can range from about 5 repeats to about 150 repeats, about 8 repeats up to about 125 repeats.

In some embodiments, the spacer comprises a photolabile linker, a fluoride ion labile lin https://app.box.com/s/ses1hgk1ffpf1yff3s2u5fvor7xr3ecx ker, or a cleavable linker. In embodiments, the spacer may comprise a benzenesulfonylethyl linker, an o-nitrobenzyl carbonate photolabile linker, a 5-methoxy-2-nitrobenzyl carbonate photolabile linker, an o-nitrophenyl-1,3-propanediol base photolabile linker, a fluoride ion labile diisopropylsilyl linker, a fluoride ion labile disiloxyl phosphoramidite linker, a NPE carbonate linker, a 9-fluorenylmethyl linker, a phthaloyl linker, an oxalyl linker, a malonic acid linker, a succinyl linker, a diglycolic acid linker, a hydroquinone-O,O′-diacetic acid (Q-linker), or a thiophospate linker. In particular embodiments, the spacer comprises a benzenesulfonylethyl linker cleavable with triethylamine/dioxane, or a spacer comprises a nonyl phenol ethoxylate (NPE) carbonate linker cleavable with 1,8-Diazabicyclo[5.4.0]undec-7-ene (DBU)/pyridine. In particular embodiments, the spacer comprises a 9-fluorenylmethyl linker or a phthaloyl linker cleavable with DBU. In some embodiments, the spacer comprises a succinic acid linked to an N-methylglycine (sarcosine) derivatized support, a succinic acid linked to 1,6-bis methylaminohexane spacer, a succinic acid linked to N-propyl polyethylene glycol Tentagel support, or a succinyl-sarcosine linkage.

The spacer may be grafted onto the solid support via an amine linkage, a secondary amine linkage, a thioether linkage, an ether linkage, a carbamate linkage, or an amide linkage, using the methods as disclosed herein.

### Agents/Labels

The solid support further comprises an agent. The agent may be attached to a solid support using a cleavable linker. In preferred embodiments, the solid support may be functionalized to permit covalent attachment of the agent and/or label. In some instances, a label (or multiple copies of the same label) and the agent are attached to the same solid support. Labels and/or agents may be attached to each other or to solid supports using cleavable linkers.

An agent can be any moiety or entity that can be associated with, including attached to, a unique label. An agent may be a single entity or it may be a plurality of entities. An agent may be a nucleic acid, a peptide, a protein, a cell, a cell lysate, a solid support, a polymer, a chemical, and the like, or an agent may be a plurality of any of the foregoing, or it may be a mixture of any of the foregoing. As an example, an agent may be nucleic acids (e.g., mRNA transcripts and/or genomic DNA fragments), solid supports such as beads or polymers, and/or proteins from a single cell or from a single cell population (e.g., a tumor or non-tumor tissue sample). An agent may also comprise a spacer as described herein.

In some embodiments, an agent is a nucleic acid. The nucleic acid agent may be single-stranded (ss) or double-stranded (ds), or it may be partially single-stranded and partially double-stranded. Nucleic acid agents include but are not limited to DNA such as genomic DNA fragments, PCR and other amplification products, RNA, cDNA, and the like. Nucleic acid agents may be fragments of larger nucleic acids such as but not limited to genomic DNA fragments. Accordingly, a portion or fragment when used in reference to a nucleotide sequence typically refers to smaller subsets of that nucleotide sequence. For example, such portions or fragments may range in size from 5 nucleotide residues to the entire nucleotide sequence minus one nucleic acid residue.

Nucleic acid sequence, nucleotide sequence, and nucleic acid molecule as used herein may interchangeably refer to an oligonucleotide or polynucleotide, and fragments or portions thereof, and to DNA or RNA of genomic or synthetic origin which may be single- or double-stranded, and represent the sense or antisense strand.

An isolated nucleic acid can refer to any nucleic acid molecule that has been removed from its natural state (e.g., removed from a cell and is, in a preferred embodiment, free of other genomic nucleic acid).

In some particular embodiments, the agent is a surface reactive nucleic acid molecule. The one or more surface reactive nucleic acid molecules may comprise an ISPCR Primer, a Barcode, a Unique Molecular Identifier (UMI) and/or a Universal Sequence. The surface reactive nucleic acid molecule may include oligonucleotides, nucleotides, analogs thereof, a molecular barcode, a Unique Molecular Identifier, a oligodT, an amplification primer, a cell type specific sequence, a pathogen-specific sequence, or a TCR specific sequence.

In some aspects, the invention provides a solid support, a population of solid support, or a composition comprising a solid support or a composition comprising a population of solid support having a surface reactive nucleic acid molecule, wherein the surface reactive nucleic acid molecule(s) comprise sequences of an ISPCR primer, a barcode, a unique molecular identifier (UMI), and a universal sequence. In some aspects, the surface reactive nucleic acid molecule(s) comprises one or more of the following: oligonucleotides, nucleotides, analogs thereof; a molecular barcode; a unique molecular identifier; a oligodT; an amplification primer; a cell type specific sequence; a pathogen-specific sequence; or a TCR specific sequence. In some aspects, the molecular barcode comprises approximately 12, 15, or 21 base pairs. In some aspects, the UMI comprises approximately 8 base pairs. In another aspect, the oligodT comprises approximately 30 base pairs.

### Barcodes and Unique Molecular Identifiers

A barcode as used herein refers to a short sequence of nucleotides (for example, DNA or RNA) that is used as an identifier for an associated molecule, such as a target molecule and/or target nucleic acid, or as an identifier of the source of an associated molecule, such as a cell-of-origin. A barcode may also refer to any unique, non-naturally occurring, nucleic acid sequence that may be used to identify the originating source of a nucleic acid fragment. Although it is not necessary to understand the mechanism of an invention, it is believed that the barcode sequence provides a high-quality individual read of a barcode associated with a single cell, a viral vector, labeling ligand (e.g., an aptamer), protein, shRNA, sgRNA or cDNA such that multiple species can be sequenced together.

In preferred embodiments, sequencing is performed using unique molecular identifiers (UMI) which may be associated with the solid supports disclosed herein. The term “unique molecular identifiers” (UMI) as used herein refers to a sequencing linker or a subtype of nucleic acid barcode used in a method that uses molecular tags to detect and quantify unique amplified products. A UMI is used to distinguish effects through a single clone from multiple clones. A clone may refer to a single mRNA or target nucleic acid to be sequenced. The UMI may also be used to determine the number of transcripts that gave rise to an amplified product, or in the case of target barcodes as described herein, the number of binding events. In preferred embodiments, the amplification is by PCR or multiple displacement amplification (MDA).

In certain embodiments, an UMI with a random sequence of between 4 and 20 base pairs is added to a template, which is amplified and sequenced. In preferred embodiments, the UMI is added to the 5′ end of the template. Sequencing allows for high resolution reads, enabling accurate detection of true variants. As used herein, a true variant will be present in every amplified product originating from the original clone as identified by aligning all products with a UMI. Each clone amplified will have a different random UMI that will indicate that the amplified product originated from that clone. Not being bound by a theory, the UMI' s are designed such that assignment to the original can take place despite up to 4-7 errors during amplification or sequencing. Not being bound by a theory, an UMI may be used to discriminate between true barcode sequences.

Unique molecular identifiers can be used, for example, to normalize samples for variable amplification efficiency. For example, in various embodiments, featuring a solid or semisolid support (for example a hydrogel bead), to which nucleic acid barcodes (for example a plurality of barcodes sharing the same sequence) are attached, each of the barcodes may be further coupled to a unique molecular identifier, such that every barcode on the particular solid or semisolid support receives a distinct unique molecule identifier. A unique molecular identifier can then be, for example, transferred to a target molecule with the associated barcode, such that the target molecule receives not only a nucleic acid barcode, but also an identifier unique among the identifiers originating from that solid or semisolid support.

A nucleic acid barcode or UMI can have a length of at least, for example, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, 60, 70, 80, 90, or 100 nucleotides, and can be in single- or double-stranded form. Target molecule and/or target nucleic acids can be labeled with multiple nucleic acid barcodes in combinatorial fashion, such as a nucleic acid barcode concatemer. Typically, a nucleic acid barcode is used to identify a target molecule and/or target nucleic acid as being from a particular discrete volume, having a particular physical property (for example, affinity, length, sequence, etc.), or having been subject to certain treatment conditions. Target molecule and/or target nucleic acid can be associated with multiple nucleic acid barcodes to provide information about all of these features (and more). Each member of a given population of UMIs, on the other hand, is typically associated with (for example, covalently bound to or a component of the same molecule as) individual members of a particular set of identical, specific (for example, discrete volume-, physical property-, or treatment condition-specific) nucleic acid barcodes. Thus, for example, each member of a set of origin-specific nucleic acid barcodes, or other nucleic acid identifier or connector oligonucleotide, having identical or matched barcode sequences, may be associated with (for example, covalently bound to or a component of the same molecule as) a distinct or different UMI.

As disclosed herein, unique nucleic acid identifiers are used to label the target molecules and/or target nucleic acids, for example origin-specific barcodes and the like. The nucleic acid identifiers, nucleic acid barcodes, can include a short sequence of nucleotides that can be used as an identifier for an associated molecule, location, or condition. In certain embodiments, the nucleic acid identifier further includes one or more unique molecular identifiers and/or barcode receiving adapters. A nucleic acid identifier can have a length of about, for example, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, 60, 70, 80, 90, or 100 base pairs (bp) or nucleotides (nt). In certain embodiments, a nucleic acid identifier can be constructed in combinatorial fashion by combining randomly selected indices (for example, about 1, 2, 3, 4, 5, 6, 7, 8, 9, or 10 indexes). Each such index is a short sequence of nucleotides (for example, DNA, RNA, or a combination thereof) having a distinct sequence. An index can have a length of about, for example, 4, 5, 6, 7, 8, 9, 10, 11, 12, 13, 14, 15, 16, 17, 18, 19, 20, 21, 22, 23, 24, or 25 bp or nt. Nucleic acid identifiers can be generated, for example, by split-pool synthesis methods, such as those described, for example, in International Patent Publication Nos. WO 2014/047556 and WO 2014/143158, each of which is incorporated by reference herein in its entirety.

One or more nucleic acid identifiers (for example a nucleic acid barcode) can be attached, or “tagged,” to a target molecule. This attachment can be direct (for example, covalent or noncovalent binding of the nucleic acid identifier to the target molecule) or indirect (for example, via an additional molecule). Such indirect attachments may, for example, include a barcode bound to a specific-binding agent that recognizes a target molecule. In certain embodiments, a barcode is attached to protein G and the target molecule is an antibody or antibody fragment. Attachment can be performed according to methods described herein.

Target molecules can be optionally labeled with multiple barcodes in combinatorial fashion (for example, using multiple barcodes bound to one or more specific binding agents that specifically recognizing the target molecule), thus greatly expanding the number of unique identifiers possible within a particular barcode pool.

In some embodiments, a nucleic acid identifier (for example, a nucleic acid barcode) may be attached to sequences that allow for amplification and sequencing (for example, SBS3 and P5 elements for Illumina sequencing). In certain embodiments, a nucleic acid barcode can further include a hybridization site for a primer (for example, a single-stranded DNA primer) attached to the end of the barcode. For example, an origin-specific barcode may be a nucleic acid including a barcode and a hybridization site for a specific primer. In particular embodiments, a set of origin-specific barcodes includes a unique primer specific barcode made, for example, using a randomized oligo type

A nucleic acid identifier can further include a unique molecular identifier and/or additional barcodes specific to, for example, a common support to which one or more of the nucleic acid identifiers are attached. Thus, a pool of target molecules can be added, for example, to a discrete volume containing multiple solid or semisolid supports (for example, beads) representing distinct treatment conditions (and/or, for example, one or more additional solid or semisolid support can be added to the discreet volume sequentially after introduction of the target molecule pool), such that the precise combination of conditions to which a given target molecule was exposed can be subsequently determined by sequencing the unique molecular identifiers associated with it.

Labeled target molecules and/or target nucleic acids associated origin-specific nucleic acid barcodes (optionally in combination with other nucleic acid barcodes as described herein) can be amplified by methods known in the art, such as polymerase chain reaction (PCR). For example, the nucleic acid barcode can contain universal primer recognition sequences that can be bound by a PCR primer for PCR amplification and subsequent high-throughput sequencing. In certain embodiments, the nucleic acid barcode includes or is linked to sequencing adapters (for example, universal primer recognition sequences) such that the barcode and sequencing adapter elements are both coupled to the target molecule. In particular examples, the sequence of the origin specific barcode is amplified, for example using PCR. In some embodiments, an origin-specific barcode further comprises a sequencing adaptor. In some embodiments, an origin-specific barcode further comprises universal priming sites.

### Barcodes Reversibly Coupled to Solid Substrate

In some embodiments, the origin-specific barcodes are reversibly coupled to a solid or semisolid substrate. In some embodiments, the origin-specific barcodes further comprise a nucleic acid capture sequence that specifically binds to the target nucleic acids and/or a specific binding agent that specifically binds to the target molecules. In specific embodiments, the origin-specific barcodes include two or more populations of origin-specific barcodes, wherein a first population comprises the nucleic acid capture sequence and a second population comprises the specific binding agent that specifically binds to the target molecules. In some examples, the first population of origin-specific barcodes further comprises a target nucleic acid barcode, wherein the target nucleic acid barcode identifies the population as one that labels nucleic acids. In some examples, the second population of origin-specific barcodes further comprises a target molecule barcode, wherein the target molecule barcode identifies the population as one that labels target molecules.

Barcode with Cleavage Sites

A nucleic acid barcode may be cleavable from a specific binding agent, for example, after the specific binding agent has bound to a target molecule. In some embodiments, the origin-specific barcode further comprises one or more cleavage sites. In some examples, at least one cleavage site is oriented such that cleavage at that site releases the origin-specific barcode from a substrate, such as a bead, for example a hydrogel bead, to which it is coupled. In some examples, at least one cleavage site is oriented such that the cleavage at the site releases the origin-specific barcode from the target molecule specific binding agent. In some examples, a cleavage site is an enzymatic cleavage site, such an endonuclease site present in a specific nucleic acid sequence. In other embodiments, a cleavage site is a peptide cleavage site, such that a particular enzyme can cleave the amino acid sequence. In still other embodiments, a cleavage site is a site of chemical cleavage.

### Barcode Adapters

In some embodiments, the target molecule is attached to an origin-specific barcode receiving adapter, such as a nucleic acid. In some examples, the origin-specific barcode receiving adapter comprises an overhang and the origin-specific barcode comprises a sequence capable of hybridizing to the overhang. A barcode receiving adapter is a molecule configured to accept or receive a nucleic acid barcode, such as an origin-specific nucleic acid barcode. For example, a barcode receiving adapter can include a single-stranded nucleic acid sequence (for example, an overhang) capable of hybridizing to a given barcode (for example, an origin-specific barcode), for example, via a sequence complementary to a portion or the entirety of the nucleic acid barcode. In certain embodiments, this portion of the barcode is a standard sequence held constant between individual barcodes. The hybridization couples the barcode receiving adapter to the barcode. In some embodiments, the barcode receiving adapter may be associated with (for example, attached to) a target molecule. As such, the barcode receiving adapter may serve as the means through which an origin-specific barcode is attached to a target molecule. A barcode receiving adapter can be attached to a target molecule according to methods known in the art. For example, a barcode receiving adapter can be attached to a polypeptide target molecule at a cysteine residue (for example, a C-terminal cysteine residue). A barcode receiving adapter can be used to identify a particular condition related to one or more target molecules, such as a cell of origin or a discreet volume of origin. For example, a target molecule can be a cell surface protein expressed by a cell, which receives a cell-specific barcode receiving adapter. The barcode receiving adapter can be conjugated to one or more barcodes as the cell is exposed to one or more conditions, such that the original cell of origin for the target molecule, as well as each condition to which the cell was exposed, can be subsequently determined by identifying the sequence of the barcode receiving adapter/barcode concatemer.

Barcode with Capture Moiety

In some embodiments, an origin-specific barcode further includes a capture moiety, covalently or non-covalently linked. In specific embodiments, a targeting probe is labeled with biotin, for instance by incorporation of biotin-16-UTP during in vitro transcription, allowing later capture by streptavidin. Other means for labeling, capturing, and detecting an origin-specific barcode include: incorporation of aminoallyl-labeled nucleotides, incorporation of sulfhydryl-labeled nucleotides, incorporation of allyl- or azide-containing nucleotides.

### Other Barcoding Embodiments

DNA barcoding is based on a relatively simple concept. For example, most eukaryote cells contain mitochondria, and mitochondrial DNA (mtDNA) has a relatively fast mutation rate, which results in significant variation in mtDNA sequences between species and, in principle, a comparatively small variance within species. A 648-bp region of the mitochondrial cytochrome c oxidase subunit 1 (CO1) gene was proposed as a potential ‘barcode’. As of 2009, databases of CO1 sequences included at least 620,000 specimens from over 58,000 species of animals, larger than databases available for any other gene. Ausubel, J., “A botanical macroscope” Proceedings of the National Academy of Sciences 106(31):12569 (2009).

Additionally, other barcoding designs and tools have been described (see e.g., Birrell et al., (2001) Proc. Natl Acad. Sci. USA 98, 12608-12613; Giaever, et al., (2002) Nature 418, 387-391; Winzeler et al., (1999) Science 285, 901-906; and Xu et al., (2009) Proc Natl Acad Sci U S A. February 17; 106(7):2289-94).

In some embodiment, the agent is associated with a unique label of the functionalized solid support or the population of functionalized solid support. Associated can refer to a relationship between the agent and the unique label such that the unique label may be used to identify the agent, identify the source or origin of the agent, identify one or more conditions to which the agent has been exposed, etc. A label that is associated with an agent may be, for example, physically attached to the agent, either directly or indirectly, or it may be in the same defined, typically physically separate, volume as the agent. A defined volume may be an emulsion droplet, a well (of for example a multiwell plate), a tube, a container, and the like. It is to be understood that the defined volume will typically contain only one agent and the label with which it is associated, although a volume containing multiple agents with multiple copies of the label is also contemplated depending on the application.

An agent may be associated with a single copy of a unique label or it may be associated with multiple copies of the same unique label including for example 2, 3, 4, 5, 6, 7, 8, 9, 10, 100, 1000, 10,000, 100,000 or more copies of the same unique label. In this context, the label is considered unique because it is different from labels associated with other, different agents.

Attachment of labels to agents may be direct or indirect. The attachment chemistry will depend on the nature of the agent and/or any derivatization or functionalization applied to the agent. For example, labels can be directly attached through covalent attachment. The label may include a moiety, which may be a non-nucleotide chemical modification, to facilitate attachment. By way of non-limiting example, the label may include methylated nucleotides, uracil bases, phosphorothioate groups, ribonucleotides, diol linkages, disulfide linkages, etc., to enable covalent attachment to an agent.

The terms coupled, connected, attached, linked, or conjugated are used interchangeably herein and encompass direct as well as indirect connection, attachment, linkage, or conjugation unless the context clearly dictates otherwise. The attachment of a ligand to a bead may be covalent or non-covalent. Attachment may be reversible or irreversible. Such attachment includes, but is not limited to, covalent bonding, ionic bonding, Van der Waals forces or friction, and the like. Methods for attaching nucleic acids to each other, as for example attaching nucleic acid labels to nucleic acid agents, are known in the art. Such methods include but are not limited to ligation, such as blunt end ligation or cohesive overhang ligation, and polymerase-mediated attachment methods (see, e.g., U.S. Pat. Nos. 7,863,058 and 7,754,429; Green and Sambrook. Molecular Cloning: A Laboratory Manual, Fourth Edition, 2012; Current Protocols in Molecular Biology, and Current Protocols in Nucleic Acid Chemistry, all of which are incorporated herein by reference).

### Adapter

In some embodiments, oligonucleotide adapters are used to attach a unique label to an agent or to a solid support. In some embodiments, an oligonucleotide adapter comprises one or more known sequences, e.g., an amplification sequence, a capture sequence, a primer sequence, and the like. In some embodiments, the adapter comprises a thymidine (T) tail overhang. Methods for producing a thymidine tail overhang are known in the art, e.g., using terminal deoxynucleotide transferase (TdT) or a polymerase that adds a thymidine overhang at the termination of polymerization. In some embodiments, the oligonucleotide adapter comprises a region that is forked.

In some embodiments, the adapter comprises a capture or detection moiety. Examples of such moieties include, but are not limited to, fluorophores, microparticles such as quantum dots (Empodocles, et al., Nature 399:126-130, 1999), gold nanoparticles (Reichert et al., Anal. Chem. 72:6025-6029, 2000), microbeads (Lacoste et al., Proc. Natl. Acad. Sci. USA 97(17):9461-9466, 2000), biotin, DNP (dinitrophenyl), fucose, digoxigenin, and other moieties known to those skilled in the art. In some embodiments, the moiety is biotin.

### Detectable Oligonucleotide Tags

The unique labels of the invention are, at least in part, nucleic acid in nature, and are generated by sequentially attaching two or more detectable oligonucleotide tags to each other. As used herein, a detectable oligonucleotide tag is an oligonucleotide that can be detected by sequencing of its nucleotide sequence and/or by detecting non-nucleic acid detectable moieties it may be attached to.

The oligonucleotide tags are typically randomly selected from a diverse plurality of oligonucleotide tags. In some instances, an oligonucleotide tag may be present once in a plurality or it may be present multiple times in a plurality. In the latter instance, the plurality of tags may be comprised of a number of subsets each comprising a plurality of identical tags. In some important embodiments, these subsets are physically separate from each other. Physical separation may be achieved by providing the subsets in separate wells of a multiwell plate or separate droplets from an emulsion. It is the random selection and thus combination of oligonucleotide tags that results in a unique label. Accordingly, the number of distinct (i.e., different) oligonucleotide tags required to uniquely label a plurality of agents can be far less than the number of agents being labeled. This is particularly advantageous when the number of agents is large (e.g., when the agents are members of a library).

The oligonucleotide tags may be detectable by virtue of their nucleotide sequence, or by virtue of a non-nucleic acid detectable moiety that is attached to the oligonucleotide such as but not limited to a fluorophore, or by virtue of a combination of their nucleotide sequence and the non-nucleic acid detectable moiety.

An oligonucleotide may be a nucleic acid such as deoxyribonucleic acid (DNA), ribonucleic acid (RNA), or DNA/RNA hybrids and includes analogs of either DNA or RNA made from nucleotide analogs known in the art (see, e.g. U.S. Patent or Patent Application Publications: U.S. Pat. Nos 7,399,845, 7,741,457, 8,022,193, 7,569,686, 7,335,765, 7,314,923, 7,335,765, and 7,816,333, US 20110009471, the entire contents of each of which are incorporated herein by reference). Oligonucleotides may be single-stranded (such as sense or antisense oligonucleotides), double-stranded, or partially single-stranded and partially double-stranded.

In some embodiments, a detectable oligonucleotide tag comprises one or more non-oligonucleotide detectable moieties. Examples of detectable moieties include, but are not limited to, fluorophores, microparticles including quantum dots (Empodocles, et al., Nature 399:126-130, 1999), gold nanoparticles (Reichert et al., Anal. Chem. 72:6025-6029, 2000), microbeads (Lacoste et al., Proc. Natl. Acad. Sci. USA 97(17):9461-9466, 2000), biotin, DNP (dinitrophenyl), fucose, digoxigenin, haptens, and other detectable moieties known to those skilled in the art. In some embodiments, the detectable moieties are quantum dots. Methods for detecting such moieties are described herein and/or are known in the art.

Thus, detectable oligonucleotide tags may be, but are not limited to, oligonucleotides comprising unique nucleotide sequences, oligonucleotides comprising detectable moieties, and oligonucleotides comprising both unique nucleotide sequences and detectable moieties.

A unique nucleotide sequence may be a nucleotide sequence that is different (and thus distinguishable) from the sequence of each detectable oligonucleotide tag in a plurality of detectable oligonucleotide tags. A unique nucleotide sequence may also be a nucleotide sequence that is different (and thus distinguishable) from the sequence of each detectable oligonucleotide tag in a first plurality of detectable oligonucleotide tags but identical to the sequence of at least one detectable oligonucleotide tag in a second plurality of detectable oligonucleotide tags. A unique sequence may differ from other sequences by multiple bases (or base pairs). The multiple bases may be contiguous or non-contiguous. Methods for obtaining nucleotide sequences (e.g., sequencing methods) are described herein and/or are known in the art.

In embodiments, detectable oligonucleotide tags comprise one or more of a ligation sequence, a priming sequence, a capture sequence, and a unique sequence. A ligation sequence is a sequence complementary to a second nucleotide sequence which allows for ligation of the detectable oligonucleotide tag to another entity comprising the second nucleotide sequence, e.g., another detectable oligonucleotide tag or an oligonucleotide adapter. A priming sequence is a sequence complementary to a primer, e.g., an oligonucleotide primer used for an amplification reaction such as but not limited to PCR. A capture sequence is a sequence capable of being bound by a capture entity. A capture entity may be an oligonucleotide comprising a nucleotide sequence complementary to a capture sequence, e.g. a second detectable oligonucleotide tag or an oligonucleotide attached to a bead. A capture entity may also be any other entity capable of binding to the capture sequence, e.g. an antibody or peptide. An index sequence is a sequence comprising a unique nucleotide sequence and/or a detectable moiety as described above. A capture entity can therefore be any molecule capable of attaching and/or binding to a nucleic acid (i.e., for example, a barcode nucleic acid). For example, a capture probe may be an oligonucleotide attached to a bead, wherein the oligonucleotide is at least partially complementary to another oligonucleotide. Alternatively, a capture probe may comprise a polyethylene glycol linker, an antibody, a polyclonal antibody, a monoclonal antibody, a Fab fragment, a biological receptor complex, an enzyme, a hormone, an antigen, and/or a fragment or portion thereof.

The nucleic acids may be bound to the support by hybridizing the capture sequence to a complementary sequence covalently attached to the support. The capture sequence (also referred to as a universal capture sequence) is a nucleic acid sequence complementary to a sequence attached to a support that may dually serve as a universal primer. In some aspects, the universal primer sequence is synthesized at the 3′ end of the oligonucleotide sequences bound to the solid support to enable stoichiometric addition of diverse oligonucleotide capture sequences to mRNA capture beads. In an aspect, photocleavable linkers may be incorporated within the oligonucleotide sequences bound to the solid support.

A label or detectable label can refer to any composition detectable by spectroscopic, photochemical, biochemical, immunochemical, electrical, optical or chemical means. Such labels include biotin for staining with labeled streptavidin conjugate, magnetic beads (e.g., Dynabeads®), fluorescent dyes (e.g., fluorescein, texas red, rhodamine, green fluorescent protein, and the like), radiolabels (e.g., 3H, 125I, 35S, 14C, or 32P), enzymes (e.g., horse radish peroxidase, alkaline phosphatase and others commonly used in an ELISA), and calorimetric labels such as colloidal gold or colored glass or plastic (e.g., polystyrene, polypropylene, latex, etc.) beads. Patents teaching the use of such labels include, but are not limited to, U.S. Pat. Nos. 3,817,837; 3,850,752; 3,939,350; 3,996,345; 4,277,437; 4,275,149; and 4,366,241 (all herein incorporated by reference). The labels contemplated in the present invention may be detected by many methods. For example, radiolabels may be detected using photographic film or scintillation counters, fluorescent markers may be detected using a photodetector to detect emitted light. Enzymatic labels are typically detected by providing the enzyme with a substrate and detecting, the reaction product produced by the action of the enzyme on the substrate, and calorimetric labels are detected by simply visualizing the colored label. Examples of the labeling substance which may be employed include labeling substances known to those skilled in the art, such as fluorescent dyes, enzymes, coenzymes, chemiluminescent substances, and radioactive substances. Specific examples include radioisotopes (e.g., 32P, 14C, 125I, 3H, and 131I), fluorescein, rhodamine, dansyl chloride, umbelliferone, luciferase, peroxidase, alkaline phosphatase, β-galactosidase, β-glucosidase, horseradish peroxidase, glucoamylase, lysozyme, saccharide oxidase, microperoxidase, biotin, and ruthenium. In the case where biotin is employed as a labeling substance, preferably, after addition of a biotin-labeled antibody, streptavidin bound to an enzyme (e.g., peroxidase) is further added. Advantageously, the label is a fluorescent label. Examples of fluorescent labels include, but are not limited to, Atto dyes, 4-acetamido-4′-isothiocyanatostilbene-2,2′disulfonic acid; acridine and derivatives: acridine, acridine isothiocyanate; 5-(2′-aminoethyl)aminonaphthalene-1-sulfonic acid (EDANS); 4-amino-N-[3-vinyl sulfonyl)phenyl]naphthalimide-3 ,5 disulfonate; N-(4-anilino-1-naphthyl)maleimide; anthranilamide; BODIPY; Brilliant Yellow; coumarin and derivatives; coumarin, 7-amino-4-methylcoumarin (AMC, Coumarin 120), 7-amino-4-trifluoromethylcouluarin (Coumaran 151); cyanine dyes; cyanosine; 4′,6-diaminidino-2-phenylindole (DAPI); 5′5″-dibromopyrogallol-sulfonaphthalein (Bromopyrogallol Red); 7-diethylamino-3-(4′-isothiocyanatophenyl)-4-methylcoumarin; diethylenetriamine pentaacetate; 4,4′-diisothiocyanatodihydro-stilbene-2,2′-disulfonic acid; 4,4′-diisothiocyanatostilbene-2,2′-disulfonic acid; 5-[dimethylamino]naphthalene-1-sulfonyl chloride (DNS, dansylchloride); 4-dimethylaminophenylazophenyl-4′-isothiocyanate (DABITC); eosin and derivatives; eosin, eosin isothiocyanate, erythrosin and derivatives; erythrosin B, erythrosin, isothiocyanate; ethidium; fluorescein and derivatives; 5-carboxyfluorescein (FAM), 5-(4,6-dichlorotriazin-2-yl)aminofluorescein (DTAF), 2′,7′-dimethoxy-4′5′-dichloro-6-carboxyfluorescein, fluorescein, fluorescein isothiocyanate, QFITC, (XRITC); fluorescamine; IR144; IR1446; Malachite Green isothiocyanate; 4-methylumbelliferoneortho cresolphthalein; nitrotyrosine; pararosaniline; Phenol Red; B-phycoerythrin; o-phthaldialdehyde; pyrene and derivatives: pyrene, pyrene butyrate, succinimidyl 1-pyrene; butyrate quantum dots; Reactive Red 4 (Cibacron™ Brilliant Red 3B-A) rhodamine and derivatives: 6-carboxy-X-rhodamine (ROX), 6-carboxyrhodamine (R6G), lissamine rhodamine B sulfonyl chloride rhodamine (Rhod), rhodamine B, rhodamine 123, rhodamine X isothiocyanate, sulforhodamine B, sulforhodamine 101, sulfonyl chloride derivative of sulforhodamine 101 (Texas Red); N,N,N′,N′ tetramethyl-6-carboxyrhodamine (TAN/IRA); tetramethyl rhodamine; tetramethyl rhodamine isothiocyanate (TRITC); riboflavin; rosolic acid; terbium chelate derivatives; Cy3; Cy5; Cy5.5; Cy7; IRD 700; IRD 800; La Jolta Blue; phthalo cyanine; and naphthalo cyanine. A fluorescent label may be a fluorescent protein, such as blue fluorescent protein, cyan fluorescent protein, green fluorescent protein, red fluorescent protein, yellow fluorescent protein or any photoconvertible protein. Colorimetric labeling, bioluminescent labeling and/or chemiluminescent labeling may further accomplish labeling. Labeling further may include energy transfer between molecules in the hybridization complex by perturbation analysis, quenching, or electron transport between donor and acceptor molecules, the latter of which may be facilitated by double stranded match hybridization complexes. The fluorescent label may be a perylene or a terrylen. In the alternative, the fluorescent label may be a fluorescent bar code. Advantageously, the label may be light sensitive, wherein the label is light-activated and/or light cleaves the one or more linkers to release the molecular cargo. The light-activated molecular cargo may be a major light-harvesting complex (LHCII). In another embodiment, the fluorescent label may induce free radical formation.

Advantageously, agents may be uniquely labeled in a dynamic manner (see, e.g., U.S. provisional patent application Ser. No. 61/703,884 filed Sep. 21, 2012). The unique labels are, at least in part, nucleic acid in nature, and may be generated by sequentially attaching two or more detectable oligonucleotide tags to each other and each unique label may be associated with a separate agent. A detectable oligonucleotide tag may be an oligonucleotide that may be detected by sequencing of its nucleotide sequence and/or by detecting non-nucleic acid detectable moieties to which it may be attached. Oligonucleotide tags may be detectable by virtue of their nucleotide sequence, or by virtue of a non-nucleic acid detectable moiety that is attached to the oligonucleotide such as but not limited to a fluorophore, or by virtue of a combination of their nucleotide sequence and the non-nucleic acid detectable moiety. A detectable oligonucleotide tag may comprise one or more non-oligonucleotide detectable moieties. Examples of detectable moieties may include, but are not limited to, fluorophores, microparticles including quantum dots (Empodocles, et al., Nature 399:126-130, 1999), gold nanoparticles (Reichert et al., Anal. Chem. 72:6025-6029, 2000), microbeads (Lacoste et al., Proc. Natl. Acad. Sci. USA 97(17):9461-9466, 2000), biotin, DNP (dinitrophenyl), fucose, digoxigenin, haptens, and other detectable moieties known to those skilled in the art.

In some embodiments, the detectable moieties may be quantum dots. Methods for detecting such moieties are described herein and/or are known in the art. Thus, detectable oligonucleotide tags may be, but are not limited to, oligonucleotides which may comprise unique nucleotide sequences, oligonucleotides which may comprise detectable moieties, and oligonucleotides which may comprise both unique nucleotide sequences and detectable moieties. A unique label may be produced by sequentially attaching two or more detectable oligonucleotide tags to each other. The detectable tags may be present or provided in a plurality of detectable tags. The same or a different plurality of tags may be used as the source of each detectable tag may be part of a unique label. In other words, a plurality of tags may be subdivided into subsets and single subsets may be used as the source for each tag. One or more other species may be associated with the tags. In particular, nucleic acids released by a lysed cell may be ligated to one or more tags. These may include, for example, chromosomal DNA, RNA transcripts, tRNA, mRNA, mitochondrial DNA, or the like. Such nucleic acids may be sequenced or further processed according to methods disclosed herein, in addition to sequencing the tags themselves, which may yield information about the nucleic acid profile of the cells, which can be associated with the tags, or the conditions that the corresponding droplet or cell was exposed to.

### Methods for Functionalized Solid Support Synthesis

Methods of functionalizing the surface of a solid support are also provided, and include reacting a solid support having surface bearing reacting groups with an activator, so as to obtain a solid support with an activated surface comprising an activating moiety, and reacting the activated surface with a spacer compound having a first moiety that reacts with the activating moiety and optionally a second moiety comprising a functional group whereby the reacting of this step b) obtains, on the solid support, a spacer grafted-thereon, whereby the surface of the solid support is functionalized. The method of functionalization of the surface can depend on the reacting groups on the surface of the solid support. In a preferred embodiment, the solid support surface bears hydroxyl reacting groups. Regardless of the reacting groups, activating the surface reacting groups should be performed in a manner to allow the functionalization of the solid support.

Following the step of activating of the surface of the solid support, the activated surface is reacted with a spacer compound comprising a first moiety that reacts with the activating moiety on the activated surface of the solid support. The spacer may also optionally contain a second moiety comprising a functional group that upon the reacting of the activated surface with the spacer compound having a first and second moiety, the solid support comprises a spacer grated-thereon, the solid support functionalized, with the second moiety comprising a functional group that can be exposed for further reaction.

### Activating the Surface of the Solid Support

In an embodiment of the invention, a solid support, e.g., toyopearl beads or other resin containing secondary hydroxyls as well as a varying amount of primary alcohols, can be functionalized according to the methods described herein by activating the reacting groups on the surface of the solid support. Successful activation of surface reacting groups allows functionalization and synthesis of capture beads. In one embodiment, the solid support may contain surface hydroxyls, e.g., primary and/or secondary hydroxyls. The surface hydroxyls of the solid support can be activated using common reagents, for example, CNBr, CDI, tresyl chloride, and divinylsulfone.

In some preferred embodiments, activating is performed under dry conditions or non-aqueous conditions or solid phase synthesis conditions.

### Activation Using Carbonyl Diimidazole (CDI)

In one embodiment, the surface hydroxyls are activated using CDI, which is a carbonylating compound originally developed for peptide synthesis. However, it is also capable of activating carboxylates and hydroxyls on supports for the immobilization of amine-containing ligands. CDI is extremely sensitive to hydrolysis in water; so all activation procedures must be done in nonaqueous conditions using dry solvents. Typically, water-miscible solvents are used like acetone, DMSO, DMF, and DMAC. Importantly, CDI can activate supports containing both primary and secondary alcohols, making it an ideal activating reagent for toyopearl supports. When CDI is used to activate hydroxyl supports, it generates imidazolyl carbamates; these activated resins are then used to immobilize amino ligands through displacement of the imidazole leaving group on the carbonyl, resulting in the formation of an alkyl carbamate linkage. The carbamate linkage is an uncharged bond that is chemical stable. The CDI-activated hydroxyl support will react nearly exclusively with primary amines, even in the presence of secondary amines, which contrasts with other activation methods like N-hydroxysuccinimide (NETS) ester or NETS carbonate-activated supports.

### Activation Using 2-Fluoro-1-Methylpyridinium (FMP)

In one embodiment, the surface hydroxyls are activated using FMP. This is a simple process for activating primary and secondary hydroxyls on supports for coupling amine- or thiol-containing ligands. Using FMP, one can generate an intermediate reactive methylpyridinium ether group, which can be coupled to ligands in aqueous or nonaqueous conditions to give secondary amines or thioether linkages. Importantly, the leaving group in this reaction is the chromophore 1-methyl-2-pyridone which can be used to determine the activation level of an FMP-activate support. Once the activated support is generated, amine or thiol-containing ligands will react in the first several hours, resulting in at least 90% yields in terms of the amount of reactive groups that can react. Residual reactive groups can be blocked using ethanolamine, which will result in numerous hydrophilic hydroxyls being created in their place. This method is useful for our application because (1) we can monitor activation levels of the FMP-activated support via the chromophore released, and (2) there is an extensive protocol already available for activating toyopearl supports, simplifying the process.

### Activation Using Organic Sulfonyl Chlorides, Toluenesulfonyls

In one embodiment, the surface hydroxyls are activated using an organic sulfonyl chloride. In one instant, sulfonate esters are formed from the activation of hydroxyl groups on supports with tosyl chloride (TsCl) to create good leaving groups, which get displaced by amine-containing ligands. Importantly, these tosyl-activated supports can react with amines, thiols, and hydroxyls by adjusting the pH, making them useful for a variety of synthesis strategies. This strategy has been used extensively in the context of antibody chemistry, specifically to couple antibodies onto PEG-modified particles for use in immunoassays (Chen A., Kozak D., Battersby B., Forrest R., Scholler N., Urban N., Trau M. 2009 Langmuir 25(23): 13510-13515.

Reacting with a Spacer

In an embodiment of the invention, the activated solid support is subsequently functionalized by attachment to a spacer, e.g., PEG spacer. Homo or hetero-functional PEG spacer can be used. Homo-functional PEG is when a PEG molecule contains the same functional group on both ends, e.g., both ends contain an amine group or both ends contain a thiol group. Hetero-functional PEG is when a PEG molecule contains different functional groups on each end, e.g., one end contains a primary amine and the other end contain a thiol.

In an exemplary embodiment, the spacer may be a primary amine containing ligand that can react readily with the activated solid support. In some embodiments, the solid support is activated in a manner such that the addition of the primary amine-containing spacer will react almost exclusively with primary amines to produce a ligand immobilized on the solid support. The ligand can be the amine functionalized PEG spacer. The step of reacting with a spacer can be performed under aqueous or nonaqueous conditions. In a preferred embodiment, the spacer is covalently bound to the solid support. When the solid support has been tosyl-activated, in some embodiments, the support is reacted with a thiol-containing ligand, an amine-containing ligand, or a hydroxyl containing ligand to provide a solid support with a spacer grafted thereon by a thioether linkage, a secondary amine linkage, or an ether linkage, respectively.

In some embodiments, the first moiety comprises NH₂, the second moiety comprises SH, OH or phenyl, and the spacer comprises a PEG; or the first moiety-spacer-second moiety comprises: X—(Y)ₙ—Z, wherein X is a thiol, a hydroxyl, an amine, or a carboxyl, Y is PEG or a methylene group, and Z is a thiol, a hydroxyl, an amine, or a carboxyl, and wherein n is an integer between 1 to 30.

### Attaching a Linker

In an aspect of the embodiment, a linker may optionally be attached to the PEG before or after attaching the spacer to the solid support. In some aspects, the linker comprises acrydite. In some aspects, the linker comprises a photolabile, a halide ion labile, or a cleavable linker described herein. The initial synthesis step (attachment of PEG spacer and optionally acrydite) is optimized to ensure that errors do not propagate in the downstream processes and that maximum bead surface area is available for a robust capture efficiency.

Reacting with a Nucleic Acid Molecule

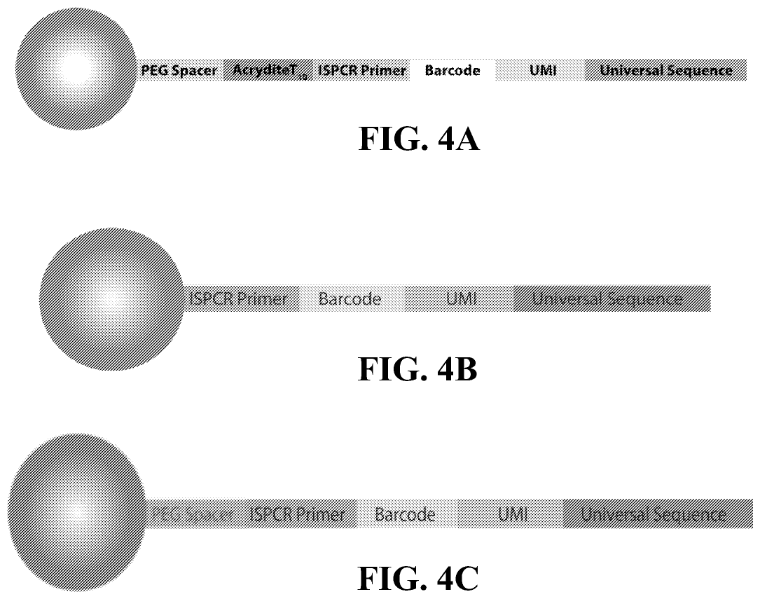

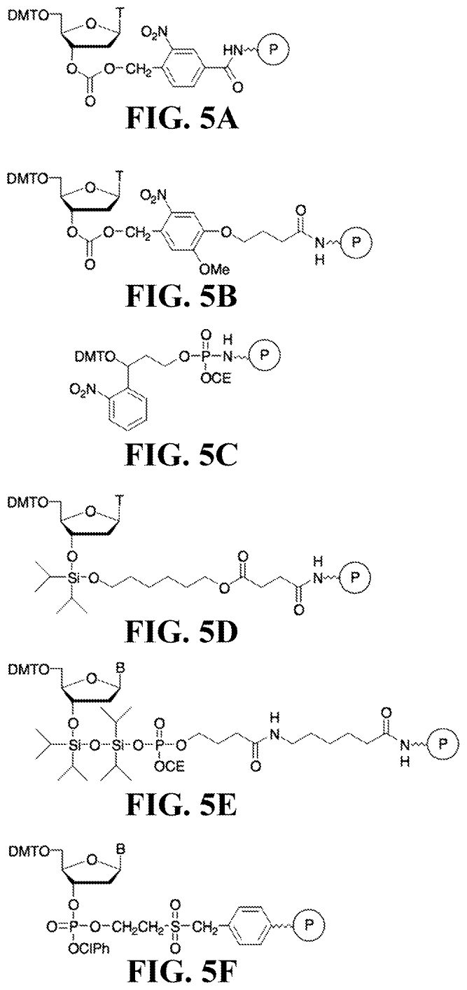

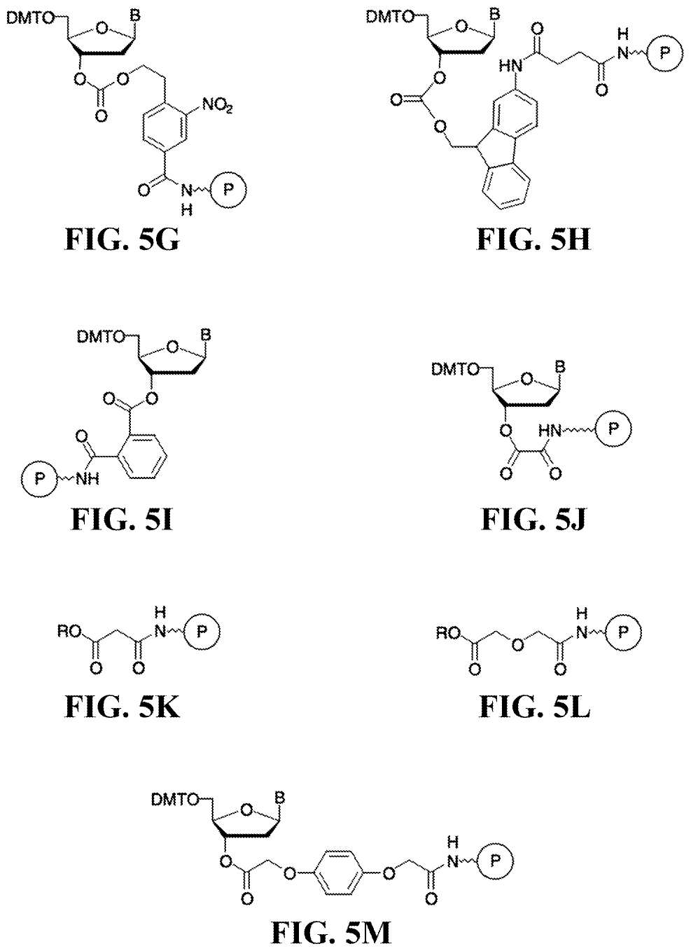

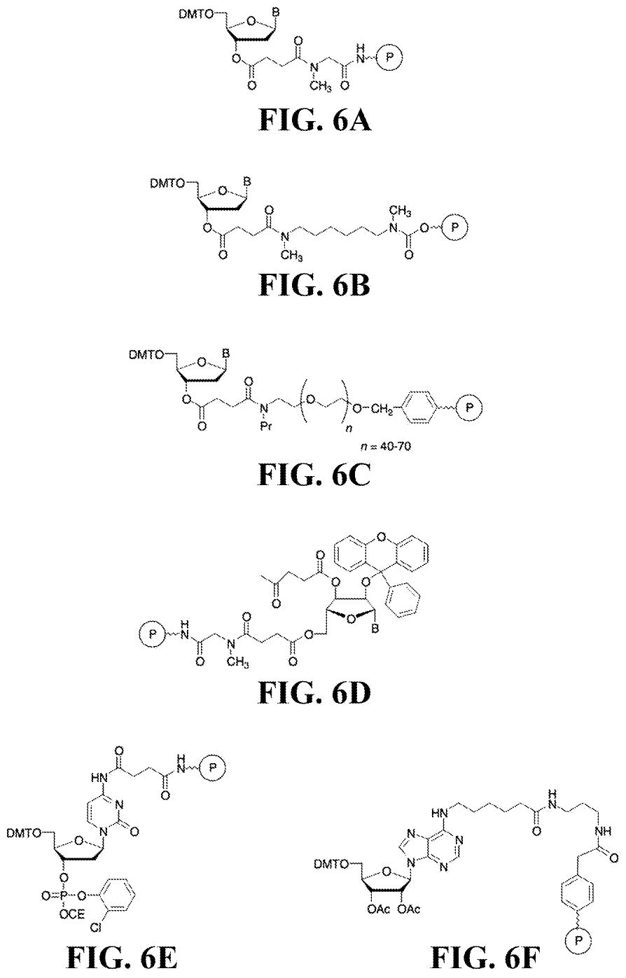

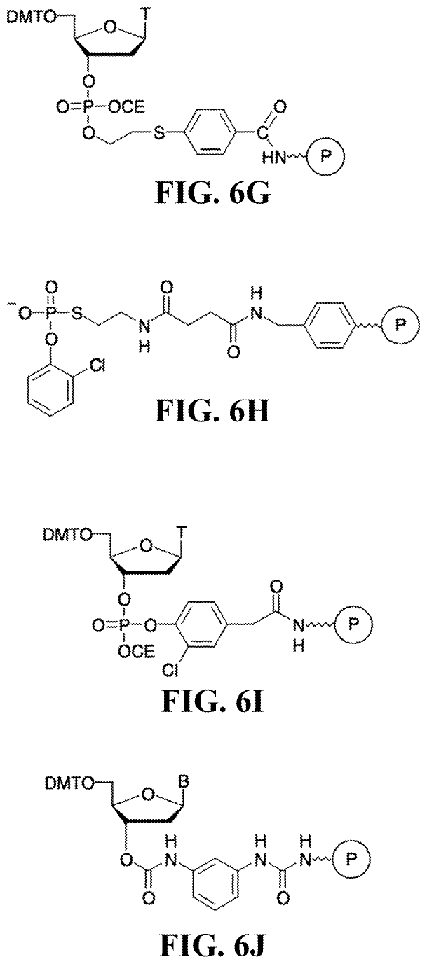

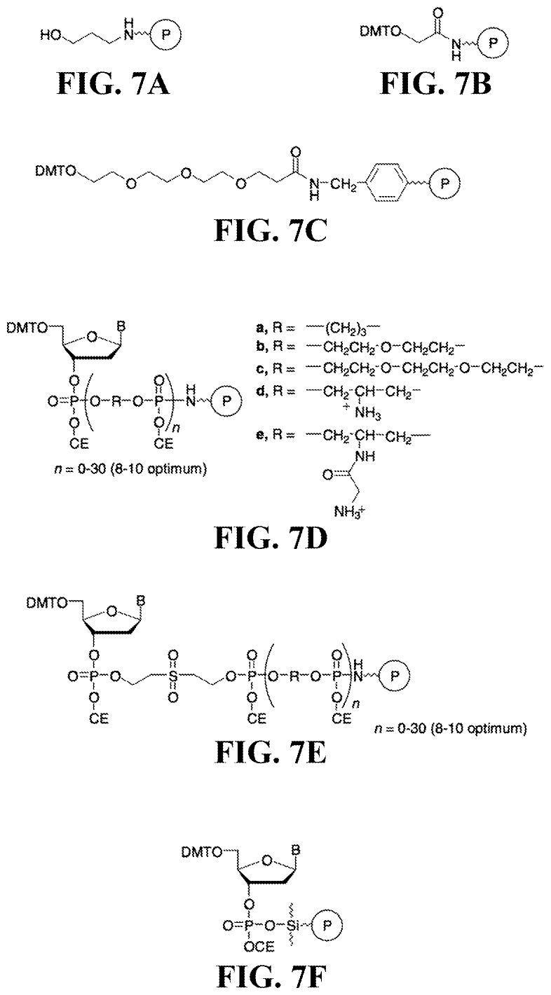

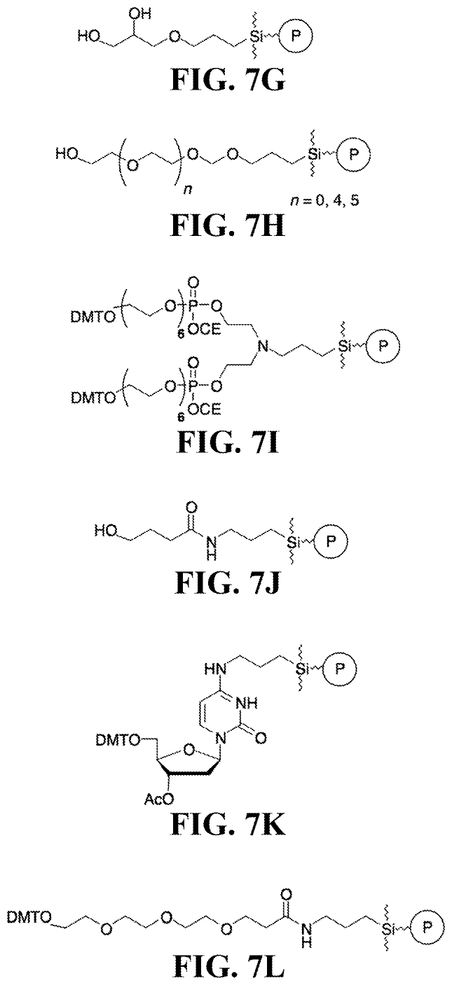

After successful attachment of the spacer, and optionally attachment of an acrydite linker to the solid support, synthesis of the ISPCR primer, barcode, and UMI, and universal sequence can be performed (FIG. 4).

The libraries of unique labels may be synthesized separately from an agent and then associated with the agent post-synthesis. Alternatively, unique labels may be synthesized in real-time, e.g., while associated with the agent. This means, in some instances, that synthesis of the label may occur while an agent is being exposed to one or more conditions such that one or more steps of the syntheses disclosed herein may be performed in different order, or simultaneously. In an embodiment of the invention, the bead synthesized by the method described herein has a higher capture efficiency compared to the capture efficiency of a commercially available bead.

### Oligonucleotide Syntheses

In some preferred embodiments, an oligonucleotide is preferably attached as one or more agents on the solid support. Oligonucleotide synthesis can be performed by one of the exemplary methods discussed herein, and may be synthesized in the 5′ to 3′ direction or the 3′ to 5′ direction. Amplification can be any suitable production of additional copies of a nucleic acid sequence and is generally carried out using polymerase chain reaction. Dieffenbach C. W. and G. S. Dveksler (1995) In: PCR Primer, a Laboratory Manual, Cold Spring Harbor Press, Plainview, N.Y. Polymerase chain reaction (“PCR”) refers to the method of K. B. Mullis U.S. Pat. Nos. 4,683,195 and 4,683,202, herein incorporated by reference, which describe a method for increasing the concentration of a segment of a target sequence in a mixture of genomic DNA without cloning or purification. The length of the amplified segment of the desired target sequence is determined by the relative positions of two oligonucleotide primers with respect to each other, and therefore, this length is a controllable parameter. By virtue of the repeating aspect of the process, the method is referred to as the “polymerase chain reaction” (hereinafter “PCR”). Because the desired amplified segments of the target sequence become the predominant sequences (in terms of concentration) in the mixture, they are said to be “PCR amplified”. With PCR, it is possible to amplify a single copy of a specific target sequence in genomic DNA to a level detectable by several different methodologies (e.g., hybridization with a labeled probe; incorporation of biotinylated primers followed by avidin-enzyme conjugate detection; incorporation of 32P-labeled deoxynucleotide triphosphates, such as dCTP or dATP, into the amplified segment). In addition to genomic DNA, any oligonucleotide sequence can be amplified with the appropriate set of primer molecules. In particular, the amplified segments created by the PCR process itself are, themselves, efficient templates for subsequent PCR amplifications.

A probe typically refers to an oligonucleotide (i.e., a sequence of nucleotides), whether occurring naturally as in a purified restriction digest or produced synthetically, recombinantly or by PCR amplification, which is capable of hybridizing to another oligonucleotide of interest. A probe may be single-stranded or double-stranded. Probes are useful in the detection, identification and isolation of particular gene sequences. It is contemplated that any probe used in the present invention will be labeled with any reporter molecule, so that it is detectable in any detection system, including, but not limited to enzyme (e.g., ELISA, as well as enzyme-based histochemical assays), fluorescent, radioactive, and luminescent systems. It is not intended that the present invention be limited to any particular detection system or label.

Purified or isolated may refer to a component of a composition that has been subjected to treatment (i.e., for example, fractionation) to remove various other components. Where the term substantially purified is used, this designation will refer to a composition in which a nucleic acid sequence forms the major component of the composition, such as constituting about 50%, about 60%, about 70%, about 80%, about 90%, about 95% or more of the composition (i.e., for example, weight/weight and/or weight/volume). Purified to homogeneity is used to include compositions that have been purified to apparent homogeneity such that there is single nucleic acid species (i.e., for example, based upon SDS-PAGE or HPLC analysis). A purified composition is not intended to mean that some trace impurities may remain. Substantially purified refers to molecules, such as nucleic acid sequences, that are removed from their natural environment, isolated or separated, and are at least 60% free, preferably 75% free, and more preferably 90% free from other components with which they are naturally associated. An isolated polynucleotide is therefore a substantially purified polynucleotide.

DNA molecules are said to have “5′ ends” and “3′ ends” because mononucleotides are reacted to make oligonucleotides in a manner such that the 5′ phosphate of one mononucleotide pentose ring is attached to the 3′ oxygen of its neighbor in one direction via a phosphodiester linkage. Therefore, an end of an oligonucleotide is referred to as the “5′ end” if its 5′ phosphate is not linked to the 3′ oxygen of a mononucleotide pentose ring. An end of an oligonucleotide is referred to as the “3′ end” if its 3′ oxygen is not linked to a 5′ phosphate of another mononucleotide pentose ring. As used herein, a nucleic acid sequence, even if internal to a larger oligonucleotide, also may be said to have 5′ and 3′ ends. In either a linear or circular DNA molecule, discrete elements are referred to as being “upstream” or 5′ of the “downstream” or 3′ elements. This terminology reflects the fact that transcription proceeds in a 5′ to 3′ fashion along the DNA strand. The promoter and enhancer elements which direct transcription of a linked gene are generally located 5′ or upstream of the coding region. However, enhancer elements can exert their effect even when located 3′ of the promoter element and the coding region. Transcription termination and polyadenylation signals are located 3′ or downstream of the coding region. Advantageously, the methods disclosed herein allow for the production of solid supports comprising nucleic acid molecules in a 5′ to 3′ orientation or in a 3′ to 5′ orientation.

A poly A site or poly A sequence as used herein denotes a DNA sequence which directs both the termination and polyadenylation of the nascent RNA transcript. Efficient polyadenylation of the recombinant transcript is desirable as transcripts lacking a poly A tail are unstable and are rapidly degraded. The poly A signal utilized in an expression vector may be heterologous or endogenous. An endogenous poly A signal is one that is found naturally at the 3′ end of the coding region of a given gene in the genome. A heterologous poly A signal is one which is isolated from one gene and placed 3′ of another gene. Efficient expression of recombinant DNA sequences in eukaryotic cells involves expression of signals directing the efficient termination and polyadenylation of the resulting transcript. Transcription termination signals are generally found downstream of the polyadenylation signal and are a few hundred nucleotides in length.

Nucleic acid molecule encoding, DNA sequence encoding, and DNA encoding may interchangeably refer to the order or sequence of deoxyribonucleotides along a strand of deoxyribonucleic acid. The order of these deoxyribonucleotides determines the order of amino acids along the polypeptide (protein) chain. The DNA sequence thus codes for the amino acid sequence.

### Solid Phase Phosphoramidite Chemistry

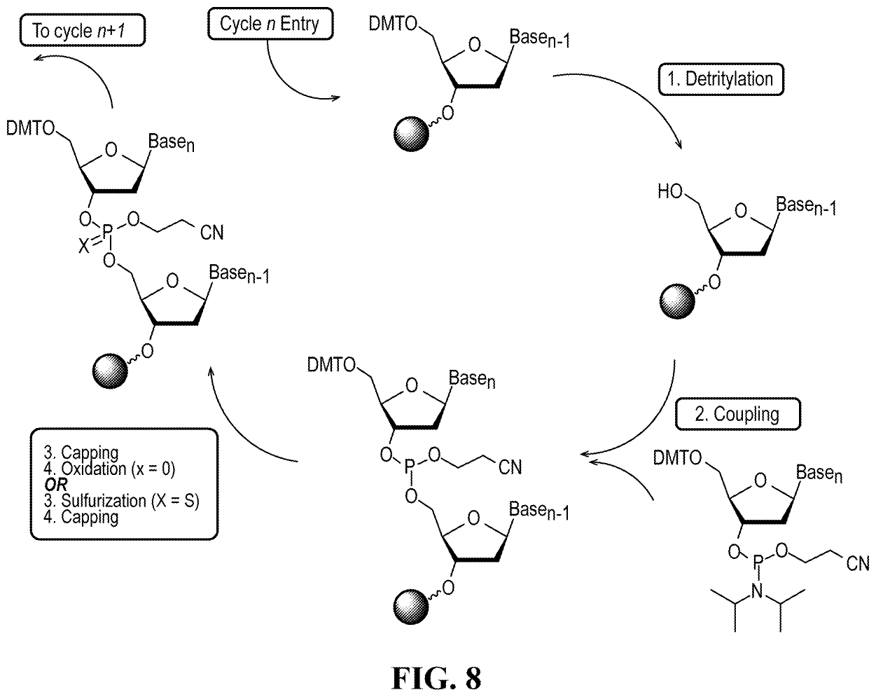

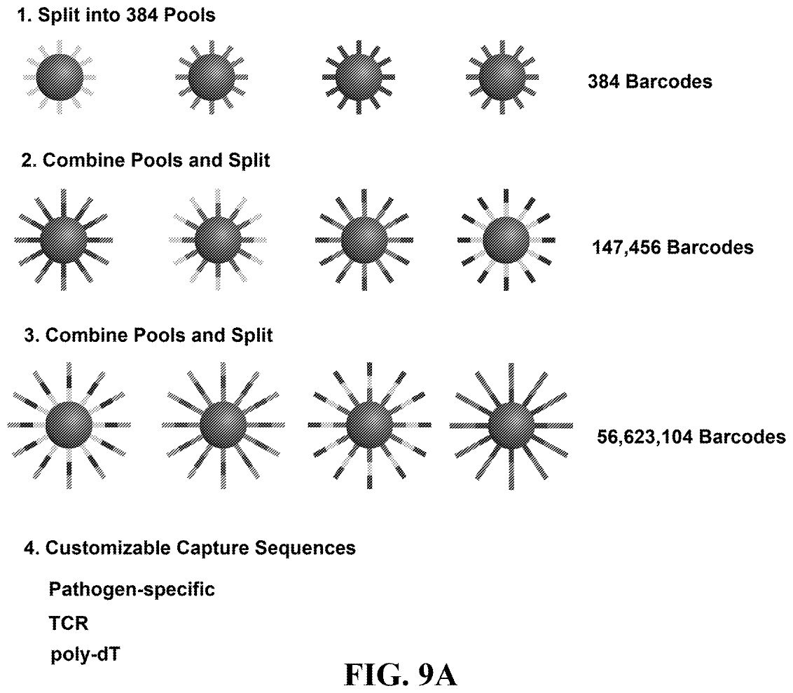

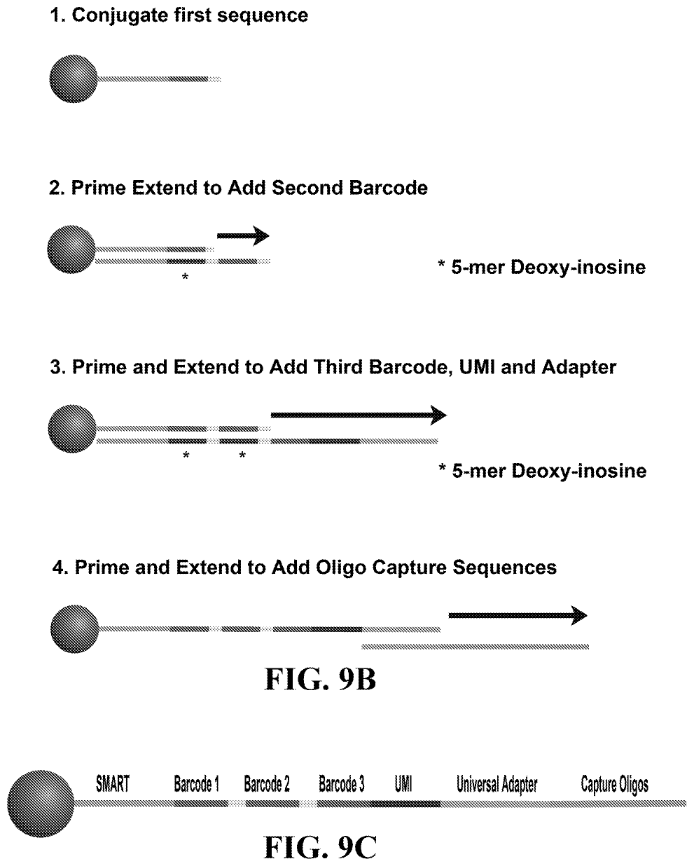

The invention provides a method for synthesizing oligonucleotides using the functionalized solid support described herein. In some aspects of the invention, the invention provides a method for synthesizing oligonucleotides by solid-phase phosphoramidite chemistry, analogous to the reaction described in Kosuri S, Church G M. Large-scale de novo DNA synthesis: technologies and applications. Nat Methods. 2014 May; 11(5):499⁻507. The method involves elongating a chain of nucleotide from 3′ end to the 5′ end. (See FIG. 8). Phosphoramidite chemistry can be used to synthesize oligos up to 100 nucleotides. The synthesized oligonucleotide may serve as a primer for amplification or an oligonucleotide probe. In some aspects, the synthesized oligonucleotide hybridizes to a primer sequence on the functionalized solid support for sequence extension reaction. In an embodiment, the oligonucleotide is synthesized in the 5′ to 3′ direction. In some aspects, single-cell transcriptomes attached to microparticles (STAMPS) are generated.

### Ligation-Mediated Method

The invention provides a method for synthesizing oligonucleotide using the functionalized solid support by a ligation method. In some aspects, the functionalized solid support is coupled with streptavidin to bind to biotinylated oligos. Fragments of oligos are ligated together using ligase, e.g., T4 DNA ligase. The ligation method is analogous to the method described in Borodina et al., Ligation-based synthesis of oligonucleotides with block structure. Anal Biochem. 2003 Jul. 15; 318(2):309-13; Kuhn et al., Template-independent ligation of single-stranded DNA by T4 DNA ligase. FEBS J. 2005 December; 272(23):5991-6000; Pengpumkiat et al., Rapid Synthesis of a Long Double-Stranded Oligonucleotide from a Single-Stranded Nucleotide Using Magnetic Beads and an Oligo Library, Plos One, 2016, 11(3):e0149774.

### Hybridization and Extension Method

The invention provides a method for synthesizing oligonucleotide using the functionalized solid support by hybridization and extension method. In some aspects, the method comprises adapter-tagged competitive PCR (ATAC PCR).

The term “tagmentation” refers to a step in the Assay for Transposase Accessible Chromatin using sequencing (ATAC-seq) as described. (See, Buenrostro, J. D., Giresi, P. G., Zaba, L. C., Chang, H. Y., Greenleaf, W. J., Transposition of native chromatin for fast and sensitive epigenomic profiling of open chromatin, DNA-binding proteins and nucleosome position. Nature methods 2013; 10 (12): 1213-1218). Specifically, a hyperactive Tn5 transposase loaded in vitro with adapters for high-throughput DNA sequencing, can simultaneously fragment and tag a genome with sequencing adapters. In one embodiment the adapters are compatible with the methods described herein.

In certain embodiments, tagmentation is used to introduce adaptor sequences to genomic DNA in regions of accessible chromatin (e.g., between individual nucleosomes) (see, e.g., US20160208323A1; US20160060691A1; WO2017156336A1; and Cusanovich, D. A., Daza, R., Adey, A., Pliner, H., Christiansen, L., Gunderson, K. L., Steemers, F. J., Trapnell, C. & Shendure, J. Multiplex single-cell profiling of chromatin accessibility by combinatorial cellular indexing. Science. 2015 May 22; 348(6237):910-4. doi: 10.1126/science.aab1601. Epub 2015 May 7). In certain embodiments, tagmentation is applied to bulk samples or to single cells in discrete volumes. In an embodiment, the functionalized solid support comprises a primer specific to a DNA target sequence. The DNA target sequence is then hybridized to the primer and one or more DNA polymerases is used for complementary strand extension. After completion of PCR, the solid supports are separated and recovered, and DNA fragment species trapped on the surfaces of the solid supports are also separated and recovered.

As used herein, the terms complementary or complementarity are used in reference to polynucleotides and oligonucleotides (which are interchangeable terms that refer to a sequence of nucleotides) related by the base-pairing rules. For example, the sequence “C-A-G-T,” is complementary to the sequence “G-T-C-A.” Complementarity can be partial or total, with partial complementarity where one or more nucleic acid bases is not matched according to the base pairing rules, and total or complete complementarity between nucleic acids is where each and every nucleic acid base is matched with another base under the base pairing rules. The degree of complementarity between nucleic acid strands has significant effects on the efficiency and strength of hybridization between nucleic acid strands. This is of particular importance in amplification reactions, as well as detection methods which depend upon binding between nucleic acids.

Hybridization is used in reference to the pairing of complementary nucleic acids using any process by which a strand of nucleic acid joins with a complementary strand through base pairing to form a hybridization complex. Hybridization and the strength of hybridization (i.e., the strength of the association between the nucleic acids) is impacted by such factors as the degree of complementarity between the nucleic acids, stringency of the conditions involved, the Tm of the formed hybrid, and the G:C ratio within the nucleic acids. Hybridization complex refers to a complex formed between two nucleic acid sequences by virtue of the formation of hydrogen bonds between complementary G and C bases and between complementary A and T bases; these hydrogen bonds may be further stabilized by base stacking interactions. The two complementary nucleic acid sequences hydrogen bond in an antiparallel configuration. A hybridization complex may be formed in solution (e.g., C0 t or R0 t analysis) or between one nucleic acid sequence present in solution and another nucleic acid sequence immobilized to a solid support (e.g., a nylon membrane or a nitrocellulose filter as employed in Southern and Northern blotting, dot blotting or a glass slide as employed in in situ hybridization, including FISH (fluorescent in situ hybridization)).

An amplifiable nucleic acid is used in reference to nucleic acids which may be amplified by any amplification method, which will usually comprise sample template. A sample template can refer to nucleic acid originating from a sample which is analyzed for the presence of a target sequence of interest. In contrast, a background template is used in reference to nucleic acid other than sample template which may or may not be present in a sample. Background template is most often inadvertent. It may be the result of carryover, or it may be due to the presence of nucleic acid contaminants sought to be purified away from the sample. For example, nucleic acids from organisms other than those to be detected may be present as background in a test sample. The present invention may include barcoding with the functionalized solid support, as discussed herein. Barcoding may be performed based on any of the compositions or methods disclosed in patent publication WO 2014047561 A1, Compositions and methods for labeling of agents, incorporated herein in its entirety. In certain embodiments barcoding uses an error correcting scheme (T. K. Moon, Error Correction Coding: Mathematical Methods and Algorithms (Wiley, New York, ed. 1, 2005)). Not being bound by a theory, amplified sequences from single cells can be sequenced together and resolved based on the barcode associated with each cell.

In some preferred embodiments, the UMI is added to the 5′ end of the template. When nucleic acid barcodes (for example a plurality of barcodes sharing the same sequence) are attached to the solid support, each of the barcodes may be further coupled to a unique molecular identifier, such that every barcode on the particular solid or semisolid support receives a distinct unique molecule identifier. A unique molecular identifier can then be, for example, transferred to a target molecule with the associated barcode, such that the target molecule receives not only a nucleic acid barcode, but also an identifier unique among the identifiers originating from that solid or semisolid support.

Target molecule and/or target nucleic acids can be labeled with multiple nucleic acid barcodes in combinatorial fashion, such as a nucleic acid barcode concatemer. Typically, a nucleic acid barcode is used to identify a target molecule and/or target nucleic acid as being from a particular discrete volume, having a particular physical property (for example, affinity, length, sequence, etc.), or having been subject to certain treatment conditions. Target molecule and/or target nucleic acid can be associated with multiple nucleic acid barcodes to provide information about all of these features (and more). Each member of a given population of UMIs, on the other hand, is typically associated with (for example, covalently bound to or a component of the same molecule as) individual members of a particular set of identical, specific (for example, discreet volume-, physical property-, or treatment condition-specific) nucleic acid barcodes. Thus, for example, each member of a set of origin-specific nucleic acid barcodes, or other nucleic acid identifier or connector oligonucleotide, having identical or matched barcode sequences, may be associated with (for example, covalently bound to or a component of the same molecule as) a distinct or different UMI.

As disclosed herein, unique nucleic acid identifiers are used to label the target molecules and/or target nucleic acids, for example origin-specific barcodes and the like. Nucleic acid identifiers can be generated, for example, by split-pool synthesis methods, such as those described, for example, in International Patent Publication Nos. WO 2014/047556 and WO 2014/143158, each of which is incorporated by reference herein in its entirety.

One or more nucleic acid identifiers (for example a nucleic acid barcode) can be attached, or “tagged,” to a target molecule. This attachment can be direct (for example, covalent or noncovalent binding of the nucleic acid identifier to the target molecule) or indirect (for example, via an additional molecule). Such indirect attachments may, for example, include a barcode bound to a specific-binding agent that recognizes a target molecule. In certain embodiments, a barcode is attached to protein G and the target molecule is an antibody or antibody fragment. Attachment of a barcode to target molecules (for example, proteins and other biomolecules) can be performed using standard methods well known in the art. For example, barcodes can be linked via cysteine residues (for example, C-terminal cysteine residues). In other examples, barcodes can be chemically introduced into polypeptides (for example, antibodies) via a variety of functional groups on the polypeptide using appropriate group-specific reagents (see for example www.drmr.com/abcon). In certain embodiments, barcode tagging can occur via a barcode receiving adapter associate with (for example, attached to) a target molecule, as described herein.

Target molecules can be optionally labeled with multiple barcodes in combinatorial fashion (for example, using multiple barcodes bound to one or more specific binding agents that specifically recognizing the target molecule), thus greatly expanding the number of unique identifiers possible within a particular barcode pool. In certain embodiments, barcodes are added to a growing barcode concatemer attached to a target molecule, for example, one at a time. In other embodiments, multiple barcodes are assembled prior to attachment to a target molecule. Compositions and methods for concatemerization of multiple barcodes are described, for example, in International Patent Publication No. WO 2014/047561, which is incorporated herein by reference in its entirety.

In some embodiments, a nucleic acid identifier (for example, a nucleic acid barcode) may be attached to sequences that allow for amplification and sequencing (for example, SBS3 and P5 elements for Illumina sequencing). In certain embodiments, a nucleic acid barcode can further include a hybridization site for a primer (for example, a single-stranded DNA primer) attached to the end of the barcode. For example, an origin-specific barcode may be a nucleic acid including a barcode and a hybridization site for a specific primer. In particular embodiments, a set of origin-specific barcodes includes a unique primer specific barcode made, for example, using a randomized oligo type

A nucleic acid identifier can further include a unique molecular identifier and/or additional barcodes specific to, for example, a common support to which one or more of the nucleic acid identifiers are attached. Thus, a pool of target molecules can be added, for example, to a discrete volume containing multiple solid or semisolid supports (for example, beads) representing distinct treatment conditions (and/or, for example, one or more additional solid or semisolid support can be added to the discreet volume sequentially after introduction of the target molecule pool), such that the precise combination of conditions to which a given target molecule was exposed can be subsequently determined by sequencing the unique molecular identifiers associated with it.

Labeled target molecules and/or target nucleic acids associated origin-specific nucleic acid barcodes (optionally in combination with other nucleic acid barcodes as described herein) can be amplified by methods known in the art, such as polymerase chain reaction (PCR). For example, the nucleic acid barcode can contain universal primer recognition sequences that can be bound by a PCR primer for PCR amplification and subsequent high-throughput sequencing. In certain embodiments, the nucleic acid barcode includes or is linked to sequencing adapters (for example, universal primer recognition sequences) such that the barcode and sequencing adapter elements are both coupled to the target molecule. In particular examples, the sequence of the origin specific barcode is amplified, for example using PCR. In some embodiments, an origin-specific barcode further comprises a sequencing adaptor. In some embodiments, an origin-specific barcode further comprises universal priming sites. A nucleic acid barcode (or a concatemer thereof), a target nucleic acid molecule (for example, a DNA or RNA molecule), a nucleic acid encoding a target peptide or polypeptide, and/or a nucleic acid encoding a specific binding agent may be optionally sequenced by any method known in the art, for example, methods of high-throughput sequencing, also known as next generation sequencing or deep sequencing. A nucleic acid target molecule labeled with a barcode (for example, an origin-specific barcode) can be sequenced with the barcode to produce a single read and/or contig containing the sequence, or portions thereof, of both the target molecule and the barcode. Exemplary next generation sequencing technologies include, for example, Illumina sequencing, Ion Torrent sequencing, 454 sequencing, SOLiD sequencing, and nanopore sequencing amongst others. In some embodiments, the sequence of labeled target molecules is determined by non-sequencing based methods. For example, variable length probes or primers can be used to distinguish barcodes (for example, origin-specific barcodes) labeling distinct target molecules by, for example, the length of the barcodes, the length of target nucleic acids, or the length of nucleic acids encoding target polypeptides. In other instances, barcodes can include sequences identifying, for example, the type of molecule for a particular target molecule (for example, polypeptide, nucleic acid, small molecule, or lipid). For example, in a pool of labeled target molecules containing multiple types of target molecules, polypeptide target molecules can receive one identifying sequence, while target nucleic acid molecules can receive a different identifying sequence. Such identifying sequences can be used to selectively amplify barcodes labeling particular types of target molecules, for example, by using PCR primers specific to identifying sequences specific to particular types of target molecules. For example, barcodes labeling polypeptide target molecules can be selectively amplified from a pool, thereby retrieving only the barcodes from the polypeptide subset of the target molecule pool.

A nucleic acid barcode can be sequenced, for example, after cleavage, to determine the presence, quantity, or other feature of the target molecule. In certain embodiments, a nucleic acid barcode can be further attached to a further nucleic acid barcode. For example, a nucleic acid barcode can be cleaved from a specific-binding agent after the specific-binding agent binds to a target molecule or a tag (for example, an encoded polypeptide identifier element cleaved from a target molecule), and then the nucleic acid barcode can be ligated to an origin-specific barcode. The resultant nucleic acid barcode concatemer can be pooled with other such concatemers and sequenced. The sequencing reads can be used to identify which target molecules were originally present in which discrete volumes.

### Barcodes Reversibly Coupled to Solid Substrate

In some embodiments, the origin-specific barcodes are reversibly coupled to a solid or semisolid substrate. In some embodiments, the origin-specific barcodes further comprise a nucleic acid capture sequence that specifically binds to the target nucleic acids and/or a specific binding agent that specifically binds to the target molecules. In specific embodiments, the origin-specific barcodes include two or more populations of origin-specific barcodes, wherein a first population comprises the nucleic acid capture sequence and a second population comprises the specific binding agent that specifically binds to the target molecules. In some examples, the first population of origin-specific barcodes further comprises a target nucleic acid barcode, wherein the target nucleic acid barcode identifies the population as one that labels nucleic acids. In some examples, the second population of origin-specific barcodes further comprises a target molecule barcode, wherein the target molecule barcode identifies the population as one that labels target molecules.

Barcode with Cleavage Sites

A nucleic acid barcode may be designed to be cleavable from a specific binding agent, for example, after the specific binding agent has bound to a target molecule. In some embodiments, the origin-specific barcode further comprises one or more cleavage sites. In some examples, at least one cleavage site is oriented such that cleavage at that site releases the origin-specific barcode from a substrate, such as a bead, for example a hydrogel bead, to which it is coupled. In some examples, at least one cleavage site is oriented such that the cleavage at the site releases the origin-specific barcode from the target molecule specific binding agent. In some examples, a cleavage site is an enzymatic cleavage site, such an endonuclease site present in a specific nucleic acid sequence. In other embodiments, a cleavage site is a peptide cleavage site, such that a particular enzyme can cleave the amino acid sequence. In still other embodiments, a cleavage site is a site of chemical cleavage.

### Barcode Adapters

In some embodiments, the target molecule is attached to an origin-specific barcode receiving adapter, such as a nucleic acid. In some examples, the origin-specific barcode receiving adapter comprises an overhang and the origin-specific barcode comprises a sequence capable of hybridizing to the overhang. A barcode receiving adapter is a molecule configured to accept or receive a nucleic acid barcode, such as an origin-specific nucleic acid barcode. For example, a barcode receiving adapter can include a single-stranded nucleic acid sequence (for example, an overhang) capable of hybridizing to a given barcode (for example, an origin-specific barcode), for example, via a sequence complementary to a portion or the entirety of the nucleic acid barcode. In certain embodiments, this portion of the barcode is a standard sequence held constant between individual barcodes. The hybridization couples the barcode receiving adapter to the barcode. In some embodiments, the barcode receiving adapter may be associated with (for example, attached to) a target molecule. As such, the barcode receiving adapter may serve as the means through which an origin-specific barcode is attached to a target molecule. A barcode receiving adapter can be attached to a target molecule according to methods known in the art. For example, a barcode receiving adapter can be attached to a polypeptide target molecule at a cysteine residue (for example, a C-terminal cysteine residue). A barcode receiving adapter can be used to identify a particular condition related to one or more target molecules, such as a cell of origin or a discreet volume of origin. For example, a target molecule can be a cell surface protein expressed by a cell, which receives a cell-specific barcode receiving adapter. The barcode receiving adapter can be conjugated to one or more barcodes as the cell is exposed to one or more conditions, such that the original cell of origin for the target molecule, as well as each condition to which the cell was exposed, can be subsequently determined by identifying the sequence of the barcode receiving adapter/barcode concatemer.

Barcode with Capture Moiety

In some embodiments, an origin-specific barcode further includes a capture moiety, covalently or non-covalently linked. Thus, in some embodiments the origin-specific barcode, and anything bound or attached thereto, that include a capture moiety are captured with a specific binding agent that specifically binds the capture moiety. In some embodiments, the capture moiety is adsorbed or otherwise captured on a surface. In specific embodiments, incorporation of biotin-16-UTP during in vitro transcription may be included; other means for labeling, capturing, and detecting an origin-specific barcode include: incorporation of aminoallyl-labeled nucleotides, incorporation of sulfhydryl-labeled nucleotides, incorporation of allyl- or azide-containing nucleotides, and many other methods described in Bioconjugate Techniques (2^{nd} Ed), Greg T. Hermanson, Elsevier (2008), which is specifically incorporated herein by reference. In some embodiments, the targeting probes are covalently coupled to a solid support or other capture device prior to contacting the sample, using methods such as incorporation of aminoallyl-labeled nucleotides followed by 1-Ethyl-3-(3-dimethylaminopropyl)carbodiimide (EDC) coupling to a carboxy-activated solid support, or other methods described in Bioconjugate Techniques. In some embodiments, the specific binding agent has been immobilized for example on a solid support, thereby isolating the origin-specific barcode.

### Other Barcoding Embodiments

DNA barcoding is also a taxonomic method that uses a short genetic marker in an organism's DNA to identify it as belonging to a particular species. It differs from molecular phylogeny in that the main goal is not to determine classification but to identify an unknown sample in terms of a known classification. Kress et al., “Use of DNA barcodes to identify flowering plants” Proc. Natl. Acad. Sci. U.S.A. 102(23):8369-8374 (2005). Barcodes are sometimes used in an effort to identify unknown species or assess whether species should be combined or separated. Koch H., “Combining morphology and DNA barcoding resolves the taxonomy of Western Malagasy Liotrigona Moure, 1961” African Invertebrates 51(2): 413-421 (2010); and Seberg et al., “How many loci does it take to DNA barcode a crocus?” PLoS One 4(2):e4598 (2009). Barcoding has been used, for example, for identifying plant leaves even when flowers or fruit are not available, identifying the diet of an animal based on stomach contents or feces, and/or identifying products in commerce (for example, herbal supplements or wood). Soininen et al., “Analysing diet of small herbivores: the efficiency of DNA barcoding coupled with high-throughput pyrosequencing for deciphering the composition of complex plant mixtures” Frontiers in Zoology 6:16 (2009).

It has been suggested that a desirable locus for DNA barcoding should be standardized so that large databases of sequences for that locus can be developed. Most of the taxa of interest have loci that are sequencable without species-specific PCR primers. CBOL Plant Working Group, “A DNA barcode for land plants” PNAS 106(31):12794-12797 (2009). Further, these putative barcode loci are believed short enough to be easily sequenced with current technology. Kress et al., “DNA barcodes: Genes, genomics, and bioinformatics” PNAS 105(8):2761-2762 (2008). Consequently, these loci would provide a large variation between species in combination with a relatively small amount of variation within a species. Lahaye et al., “DNA barcoding the floras of biodiversity hotspots” Proc Natl Acad Sci USA 105(8):2923-2928 (2008).

DNA barcoding is based on a relatively simple concept. For example, most eukaryote cells contain mitochondria, and mitochondrial DNA (mtDNA) has a relatively fast mutation rate, which results in significant variation in mtDNA sequences between species and, in principle, a comparatively small variance within species. A 648-bp region of the mitochondrial cytochrome c oxidase subunit 1 (CO1) gene was proposed as a potential ‘barcode’. As of 2009, databases of CO1 sequences included at least 620,000 specimens from over 58,000 species of animals, larger than databases available for any other gene. Ausubel, J., “A botanical macroscope” Proceedings of the National Academy of Sciences 106(31):12569 (2009).

Software for DNA barcoding requires integration of a field information management system (FIMS), laboratory information management system (LIMS), sequence analysis tools, workflow tracking to connect field data and laboratory data, database submission tools and pipeline automation for scaling up to eco-system scale projects. Geneious Pro can be used for the sequence analysis components, and the two plugins made freely available through the Moorea Biocode Project, the Biocode LIMS and Genbank Submission plugins handle integration with the FIMS, the LIMS, workflow tracking and database submission.

Additionally, other barcoding designs and tools have been described (see e.g., Birrell et al., (2001) Proc. Natl Acad. Sci. USA 98, 12608-12613; Giaever, et al., (2002) Nature 418, 387-391; Winzeler et al., (1999) Science 285, 901-906; and Xu et al., (2009) Proc Natl Acad Sci U S A. February 17; 106(7):2289-94).

The present invention is useful in a scalable analysis of DNA sequencing products from the DNA microscopy amplification reaction. The scalable analysis involves two challenges, one being UMI/UEI error correction, the second being UMI position inference. UMI/UEI error correction involves sequence-clustering of N UMI or UEI sequences, where N is large. If comparisons of these N sequences were computed directly, this would require N² comparisons. A solution involves (1) reducing error correction to a clustering problem requiring only knowledge of nearest neighbors and (2) performing local similarity hashing to align UMI/UEI sequences to each other.

UMI position inference involves searching for an optimal positioning of N UMI's: a relative positioning problem that would similarly require N² comparisons per iteration of the optimization algorithm if this were computed directly. A solution involves using the Barnes-Hut algorithm [A hierarchical o (n log n) force-calculation algorithm. Nature, 324:446{449, December 1986] for reducing the N² comparisons to N log N comparisons.

Error correction relies on errors being infrequent and sequencing coverage being sufficiently high so that errors can be corrected based on a collection of reads at the same genomic locus. k-mer-based correction is an overlap-based method which can overcome base substitution errors and can also correct insertions and deletions. The present invention includes k-mer based sequencing methods, including, but not limited to a 5-mer, 15 bp barcoding strategy, a 5-mer, 20 base pair barcoding strategy, and a 3-mer, 21 base pair barcoding strategy.

In an embodiment of the invention, the functionalized solid support comprises a barcode and error-correcting barcode, which can be generated through sequential synthesis of 3 or 4 rounds of 5-mer sequences (Hamming distance=2) across 188 synthesis columns to minimize barcode collision rate.

In an embodiment of the invention, the incorporation of 2 V bases at the 3′ end of the unique molecular identifier can be used to identify sequences with truncations during synthesis. For example: UMI Sequence NNNNNNVV-polydT (SEQ ID NO:4) (30) or NNNNNNVV-TT-(SEQ ID NO:5) Universal Primer Sequence.

### Universal Priming Strategies

In some embodiments, a plurality of nucleic acid molecules being sequenced is bound to a support (e.g., functionalized solid support described herein). To immobilize the nucleic acid on a support, a capture sequence/universal priming site can be added at the 3′ and/or 5′ end of the template. The nucleic acids may be bound to the support by hybridizing the capture sequence to a complementary sequence covalently attached to the support. The capture sequence (also referred to as a universal capture sequence) is a nucleic acid sequence complementary to a sequence attached to a support that may dually serve as a universal primer. In some aspects, the universal primer sequence is synthesized at the 3′ end of the oligonucleotide sequences bound to the solid support to enable stoichiometric addition of diverse oligonucleotide capture sequences to mRNA capture beads. Subsequent priming and extension of the oligonucleotide sequence can be done using a range of suitable polymerases, e.g., Klenow, BST, T4 polymerase, etc. In some aspects, a T7 promoter sequence can be added to the priming site of the oligonucleotide sequence. In an aspect, photocleavable linkers may be incorporated within the oligonucleotide sequences bound to the solid support.

### Single Cell Profiling

Single-cell profiling is a technique that exposes inherent responses which otherwise are unable to be studied in the context of a complex, and non-uniform environment. Biological samples, such as tissue, are broken down in order to study cell types and reveal pertinent cell expression profiles. Currently, techniques used for single-cell profiling entail quantitative reverse transcription PCR (RT-qPCR) and single-cell RNA-Seq, as well as other single-cell genomic techniques. RT-qPCR provides a highly sensitive, high-throughput single-cell profiling technique with multiplexing developed to target mRNA, microRNA, non-coding RNA, and proteins. (Stahlberg, A; Kubista, M. Expert Rev. Mol. Diagn. 14(3), 323-331 (2014).

Performing studies that require data resolution at the single cell (or single molecule) level can be challenging or cost prohibitive under the best circumstances. Although techniques or instruments for single molecule or single cell analysis exist (e.g., digital polymerase chain reactions (PCR) or Fluidigm C1, respectively), none currently allows a scalable method for dynamically delivering reagents and/or appending molecular “information” to individual reactions such that a large population of reactions/assays can be processed and analyzed en masse while still maintaining the ability to partition results by individual reactions/assays. (mention is made of Mazutis, L., Gilbert, J., Ung, W. L., Weitz, D. A., Griffiths, A. D., and Heyman, J. A. (2013). Single-cell analysis and sorting using droplet-based microfluidics. Nature protocols 8, 870-891.)

Microfluidics involves micro-scale devices that handle small volumes of fluids. Because microfluidics may accurately and reproducibly control and dispense small fluid volumes, in particular volumes less than 1 μl, application of microfluidics provides significant cost-savings. The use of microfluidics technology reduces cycle times, shortens time-to-results, and increases throughput. Furthermore, incorporation of microfluidics technology enhances system integration and automation. Microfluidic reactions are generally conducted in microdroplets. The ability to conduct reactions in microdroplets depends on being able to merge different sample fluids and different microdroplets. See, e.g., US Patent Publication No. 20120219947.

Droplet microfluidics offers significant advantages for performing high-throughput screens and sensitive assays. Droplets allow sample volumes to be significantly reduced, leading to concomitant reductions in cost. Manipulation and measurement at kilohertz speeds enable up to 10⁸ discrete biological entities (including, but not limited to, individual cells or organelles) to be screened in a single day. Compartmentalization in droplets increases assay sensitivity by increasing the effective concentration of rare species and decreasing the time required to reach detection thresholds. Droplet microfluidics combines these powerful features to enable currently inaccessible high-throughput screening applications, including single-cell and single-molecule assays. See, e.g., Guo et al., Lab Chip, 2012,12, 2146-2155.

Drop-Sequence methods and apparatus provides a high-throughput single-cell RNA-Seq and/or targeted nucleic acid profiling (for example, sequencing, quantitative reverse transcription polymerase chain reaction, and the like) where the RNAs from different cells are tagged individually, allowing a single library to be created while retaining the cell identity of each read. A combination of molecular barcoding and emulsion-based microfluidics to isolate, lyse, barcode, and prepare nucleic acids from individual cells in high-throughput is used. Microfluidic devices (for example, fabricated in polydimethylsiloxane), sub-nanoliter reverse emulsion droplets. These droplets are used to co-encapsulate nucleic acids with a barcoded capture bead. Each bead, for example, is uniquely barcoded so that each drop and its contents are distinguishable. The nucleic acids may come from any source known in the art, such as for example, those which come from a single cell, a pair of cells, a cellular lysate, or a solution. The cell is lysed as it is encapsulated in the droplet. To load single cells and barcoded beads into these droplets with Poisson statistics, 100,000 to 10 million such beads are needed to barcode ˜10,000-100,000 cells.

In some aspects of the invention, the present application enables one to functionalize capture beads or prepare a population of capture beads that can be used to capture mRNA transcripts from single cells using the methods described. Mention is made of mRNA transcript capture from single cells using microfluidic devices. In a report by Walsh et al. Lab Chip, 2015, 15, 2968-2980, the disclosure relates to a microfluidic device for automatic hydrodynamic capture of single mammalian cells and subsequent immobilization and digital counting of polyadenylated mRNA molecules released from individual cells. Single HeLA cells are captured by hybridization to oligonucleotides attached on the glass surface in the device, which is visually monitored using single-molecule fluorescence imaging. Macromolecule capture

Macromolecular capture is accomplished by co-loading cells of interest and functionalized solid support for the molecule of interest into functionalized arrays. The preferred functionalized solid support for mRNA is barcoded, poly(dT) beads, for protein is base-activated such as NHS or glyoxal activated agarose beads and for genome is weak anionic exchange resins. When multiple macromolecular capture is desired, it is critical that the beads are specific to their intended target which is accomplished either by the specificity of the bead or the sequence in which the beads are activated for binding—i.e, anionic exchange resin when active will bind both DNA and RNA and some protein when active at low pH and low ionic strength, but can be held in the well in an inactive form by high pH buffer while RNA and protein bind their respective resins. Typically, the functionalized solid support or functionalized beads are loaded into the nanowell array first due to buffer requirements for efficient resin loading (low pH for protein resin and high pH for poly(dT) beads) that are toxic to cells. Once the desired combination of resins is loaded, buffer is switched to tissue culture media and cells are loaded. The nano-liter scale wells are then sealed using a track-etched polycarbonate ultrafiltration membrane using a clamp. After 30 min the clamp is removed. In normal tissue media, wells can remain sealed for >24 hours retaining any macromolecule within the volume of the nanowell while allowing exchange of small molecules from bulk solutions in which the sealed array is submerged. Crucially, the buffers can be easily changed, enabling control over the reaction conditions within the sealed nano-liter scale wells. The sealed array goes through a series of buffer exchanges and hybridizations depending on the identity and number of macromolecules that are to be captured. Once all molecules are secured to a surface, the membrane is peeled off the array. Typically, reverse transcription is the first step performed after membrane removal as mRNA is the most labile of the macromolecules. Protein is the next to be analyzed which can be accomplished directly with fluorescent antibodies. Applicants envision ultimately using DNA-barcoded antibodies to label proteins and post transcriptional modifications. The protein DNA barcodes will be released from the antibody, captured on the barcoded poly(dT) bead and reading out protein content with sequencing using the bead barcode to match it to the transcript but this has not been fully implemented yet. Captured genomic content can be queried through on array PCR, amplified on array use WGA or recovered through micromanipulation for bulk processes.

### Seq-Well

Once all desired analysis of macromolecules on the array has been completed, the barcoded beads can be recovered from the array through centrifugation or scraping them off the surface. The barcoded cDNA can undergo whole transcriptome amplification and then be sequenced in bulk. Each sequencing read can be traced back to a single cell using the bead barcode attached to each transcript during the RT reaching identical to published protocols such as DropSeq. In other instances, reverse transcription can be performed on the array. In an embodiment, the functionalized solid support or a population of functionalized solid support can be used in a high-throughput parallel single cell biochemical analysis in an array of wells or containers. (See WO/2017124101, the content of which is incorporated herein).

### Drop-Seq

The functionalized solid support described herein, including the cellulose-based solid support or bead, can be used in a droplet-based method (Drop-Seq) for single-cell nucleic acid analysis as described in WO2016/040476, the content of which is incorporated herein. Notably, the use of cellulose beads allow for super-poisson load Drop-seq devices, which is a great improvement of previous Drop-seq configuration. In one embodiment, the functionalized solid support comprises beads having a spacer, a sequence for use as a sequencing priming site, a uniform or near-uniform nucleotide or oligonucleotide sequence, a Unique Molecular Identifier which differs for each priming site; optionally an oligonucleotide redundant sequence for capturing polyadenylated mRNAs and priming reverse transcription; and optionally at least one other oligonucleotide barcode which provides an additional substrate for identification or for downstream molecular-biological reactions; wherein the uniform or near-uniform nucleotide or oligonucleotide sequence is the same across all the priming sites on any one bead, but varies among the oligonucleotides on an individual bead. The downstream molecular biological reactions are for reverse transcription of mature mRNAs; capturing specific portions of the transcriptome, priming for DNA polymerases and/or similar enzymes; or priming throughout the transcriptome or genome.

In certain embodiments, the invention involves plate based single cell RNA sequencing (see, e.g., Picelli, S. et al., 2014, “Full-length RNA-seq from single cells using Smart-seq2” Nature protocols 9, 171-181, doi:10.1038/nprot.2014.006).

In certain embodiments, the solid supports can be used in high-throughput single-cell RNA-seq and/or targeted nucleic acid profiling (for example, sequencing, quantitative reverse transcription polymerase chain reaction, and the like) where the RNAs from different cells are tagged individually, allowing a single library to be created while retaining the cell identity of each read. In this regard reference is made to Macosko et al., 2015, “Highly Parallel Genome-wide Expression Profiling of Individual Cells Using Nanoliter Droplets” Cell 161, 1202-1214; International patent application number PCT/US2015/049178, published as WO2016/040476 on Mar. 17, 2016; Klein et al., 2015, “Droplet Barcoding for Single-Cell Transcriptomics Applied to Embryonic Stem Cells” Cell 161, 1187-1201; International patent application number PCT/US2016/027734, published as WO2016168584A1 on Oct. 20, 2016; Zheng, et al., 2016, “Haplotyping germline and cancer genomes with high-throughput linked-read sequencing” Nature Biotechnology 34, 303-311; Zheng, et al., 2017, “Massively parallel digital transcriptional profiling of single cells” Nat. Commun. 8, 14049 doi: 10.1038/ncomms14049; International patent publication number WO2014210353A2; Zilionis, et al., 2017, “Single-cell barcoding and sequencing using droplet microfluidics” Nat Protoc. January; 12(1):44-73; Cao et al., 2017, “Comprehensive single cell transcriptional profiling of a multicellular organism by combinatorial indexing” bioRxiv preprint first posted online Feb. 2, 2017, doi: dx.doi.org/10.1101/104844; Rosenberg et al., 2017, “Scaling single cell transcriptomics through split pool barcoding” bioRxiv preprint first posted online Feb. 2, 2017, doi: dx.doi.org/10.1101/105163; Vitak, et al., “Sequencing thousands of single-cell genomes with combinatorial indexing” Nature Methods, 14(3):302-308, 2017; Cao, et al., Comprehensive single-cell transcriptional profiling of a multicellular organism. Science, 357(6352):661-667, 2017; and Gierahn et al., “Seq-Well: portable, low-cost RNA sequencing of single cells at high throughput” Nature Methods 14, 395-398 (2017), all the contents and disclosure of each of which are herein incorporated by reference in their entirety.

The invention involves a high-throughput single-cell RNA-Seq and/or targeted nucleic acid profiling (for example, sequencing, quantitative reverse transcription polymerase chain reaction, and the like) where the RNAs from different cells are tagged individually, allowing a single library to be created while retaining the cell identity of each read. In this regard, technology of WO2016/040476, the disclosure of which is incorporated by reference, may be used in or as to the invention. A combination of molecular barcoding and emulsion-based microfluidics to isolate, lyse, barcode, and prepare nucleic acids from individual cells in high-throughput is used. Microfluidic devices (for example, fabricated in polydimethylsiloxane), sub-nanoliter reverse emulsion droplets. These droplets are used to co-encapsulate nucleic acids with a functionalized solid support, e.g., a barcoded capture bead described herein. Each bead, for example, is uniquely barcoded so that each drop and its contents are distinguishable. The nucleic acids may come from any source known in the art, such as for example, those which come from a single cell, a pair of cells, a cellular lysate, or a solution. The cell is lysed as it is encapsulated in the droplet. To load single cells and barcoded beads into these droplets with Poisson statistics, 100,000 to 10 million such beads are needed to barcode ˜10,000-100,000 cells. In this regard there can be a single-cell sequencing library which may comprise: merging one uniquely barcoded mRNA capture microbead with a single-cell in an emulsion droplet having a diameter of 75-125 μm; lysing the cell to make its RNA accessible for capturing by hybridization onto RNA capture microbead; performing a reverse transcription either inside or outside the emulsion droplet to convert the cell's mRNA to a first strand cDNA that is covalently linked to the mRNA capture microbead; pooling the cDNA-attached microbeads from all cells; and preparing and sequencing a single composite RNA-Seq library. Accordingly, it is envisioned as to or in the practice of the invention provides that there can be a method for preparing uniquely barcoded mRNA capture microbeads, which has a unique barcode and diameter suitable for microfluidic devices which may comprise: 1) performing reverse phosphoramidite synthesis on the surface of the bead in a pool-and-split fashion, such that in each cycle of synthesis the beads are split into four reactions with one of the four canonical nucleotides (T, C, G, or A) or unique oligonucleotides of length two or more bases; 2) repeating this process a large number of times, at least six, and optimally more than twelve, such that, in the latter, there are more than 16 million unique barcodes on the surface of each bead in the pool. (See www.ncbi.nlm.nih.gov/pmc/articles/PMC206447). Likewise, in or as to the instant invention there can be an apparatus for creating a single-cell sequencing library via a microfluidic system, which may comprise: an oil-surfactant inlet which may comprise a filter and a carrier fluid channel, wherein said carrier fluid channel further may comprise a resistor; an inlet for an analyte which may comprise a filter and a carrier fluid channel, wherein said carrier fluid channel may further comprise a resistor; an inlet for mRNA capture microbeads and lysis reagent which may comprise a filter and a carrier fluid channel, wherein said carrier fluid channel may further comprise a resistor; said carrier fluid channels have a carrier fluid flowing therein at an adjustable or predetermined flow rate; wherein each said carrier fluid channels merge at a junction; and said junction being connected to a mixer, which contains an outlet for drops. Similarly, as to or in the practice of the instant invention there can be a method for creating a single-cell sequencing library which may comprise: merging one uniquely barcoded RNA capture microbead with a single-cell in an emulsion droplet having a diameter of 125 μm lysing the cell thereby capturing the RNA on the RNA capture microbead; performing a reverse transcription either after breakage of the droplets and collection of the microbeads; or inside the emulsion droplet to convert the cell's RNA to a first strand cDNA that is covalently linked to the RNA capture microbead; pooling the cDNA-attached microbeads from all cells; and preparing and sequencing a single composite RNA-Seq library; and, the emulsion droplet can be between 50-210 μm. In a further embodiment, the method wherein the diameter of the mRNA capture microbeads is from 10 μm to 95 μm.

Thus, the practice of the instant invention comprehends preparing uniquely barcoded mRNA capture microbeads, which has a unique barcode and diameter suitable for microfluidic devices which may comprise: 1) performing reverse phosphoramidite synthesis on the surface of the bead in a pool-and-split fashion, such that in each cycle of synthesis the beads are split into four reactions with one of the four canonical nucleotides (T,C,G,or A); 2) repeating this process a large number of times, at least six, and optimally more than twelve, such that, in the latter, there are more than 16 million unique barcodes on the surface of each bead in the pool. The covalent bond can be polyethylene glycol. The diameter of the mRNA capture microbeads can be from 10 p.m to 95 μm. Accordingly, it is also envisioned as to or in the practice of the invention that there can be a method for preparing uniquely barcoded mRNA capture microbeads, which has a unique barcode and diameter suitable for microfluidic devices which may comprise: 1) performing reverse phosphoramidite synthesis on the surface of the bead in a pool-and-split fashion, such that in each cycle of synthesis the beads are split into four reactions with one of the four canonical nucleotides (T, C, G, or A); 2) repeating this process a large number of times, at least six, and optimally more than twelve, such that, in the latter, there are more than 16 million unique barcodes on the surface of each bead in the pool. And, the diameter of the mRNA capture microbeads can be from 10 μm to 95 μm.

Being able to dynamically track individual cells and droplet treatments/combinations during life cycle experiments, and having an ability to create a library of emulsion droplets on demand with the further capability of manipulating the droplets through the disclosed process(es) are advantageous. The solid support compositions, including mRNA capture microbeads, described herein can be used in dynamic tracking of the droplets and create a history of droplet deployment and application in a single cell based environment.

The practice of the invention may include an apparatus for creating a composite single-cell sequencing library via a microfluidic system, which may comprise: an oil-surfactant inlet which may comprise a filter and two carrier fluid channels, wherein said carrier fluid channel further may comprise a resistor; an inlet for an analyte which may comprise a filter and two carrier fluid channels, wherein said carrier fluid channel further may comprise a resistor; an inlet for mRNA capture microbeads and lysis reagent which may comprise a carrier fluid channel; said carrier fluid channels have a carrier fluid flowing therein at an adjustable and predetermined flow rate; wherein each said carrier fluid channels merge at a junction; and said junction being connected to a constriction for droplet pinch-off followed by a mixer, which connects to an outlet for drops. The analyte may comprise a chemical reagent, a genetically perturbed cell, a protein, a drug, an antibody, an enzyme, a nucleic acid, an organelle like the mitochondrion or nucleus, a cell or any combination thereof. In an embodiment of the apparatus the analyte is a cell. In an embodiment of the apparatus the lysis reagent may comprise an anionic surfactant such as sodium lauroyl sarcosinate, or a chaotropic salt such as guanidinium thiocyanate. The filter can involve square PDMS posts; e.g., with the filter on the cell channel of such posts with sides ranging between 125-135 μm with a separation of 70-100 mm between the posts. The filter on the oil-surfactant inlet may comprise square posts of two sizes; one with sides ranging between 75-100 μm and a separation of 25-30 μm between them and the other with sides ranging between 40-50 μm and a separation of 10-15 μm. The apparatus can involve a resistor, e.g., a resistor that is serpentine having a length of 7000-9000 μm, width of 50-75 μm and depth of 100-150 mm. The apparatus can have channels having a length of 8000-12,000 μm for oil-surfactant inlet, 5000-7000 for analyte (cell) inlet, and 900-1200 μm for the inlet for microbead and lysis agent; and/or all channels having a width of 125-250 mm, and depth of 100-150 mm. The width of the cell channel can be 125-250 μm and the depth 100-150 μm. The apparatus can include a mixer having a length of 7000-9000 μm, and a width of 110-140 μm with 35-45o zig-zigs every 150 μm. The width of the mixer can be about 125 μm. The oil-surfactant can be a PEG Block Polymer, such as BIORAD™ QX200 Droplet Generation Oil. The carrier fluid can be a water-glycerol mixture.

Mixtures for use in described technologies may comprise a plurality of solid support compositions, for example, microbeads, adorned with combinations of the following elements: bead-specific oligonucleotide barcodes; additional oligonucleotide barcode sequences which vary among the oligonucleotides on an individual bead and can therefore be used to differentiate or help identify those individual oligonucleotide molecules; additional oligonucleotide sequences that create substrates for downstream molecular-biological reactions, such as oligo-dT (for reverse transcription of mature mRNAs), specific sequences (for capturing specific portions of the transcriptome, or priming for DNA polymerases and similar enzymes), or random sequences (for priming throughout the transcriptome or genome). The individual oligonucleotide molecules on the surface of any individual microbead may contain all three of these elements, and the third element may include both oligo-dT and a primer sequence. A mixture may comprise a plurality of microbeads, wherein said microbeads may comprise the following elements: at least one bead-specific oligonucleotide barcode; at least one additional identifier oligonucleotide barcode sequence, which varies among the oligonucleotides on an individual bead, and thereby assisting in the identification and of the bead specific oligonucleotide molecules; optionally at least one additional oligonucleotide sequences, which provide substrates for downstream molecular-biological reactions. A mixture may comprise at least one oligonucleotide sequence(s), which provide for substrates for downstream molecular-biological reactions. In a further embodiment the downstream molecular biological reactions are for reverse transcription of mature mRNAs; capturing specific portions of the transcriptome, priming for DNA polymerases and/or similar enzymes; or priming throughout the transcriptome or genome. The mixture may involve additional oligonucleotide sequence(s) which may comprise a oligio-dT sequence. The mixture further may comprise the additional oligonucleotide sequence which may comprise a primer sequence. The mixture may further comprise the additional oligonucleotide sequence which may comprise a oligo-dT sequence and a primer sequence.

Examples of the labeling substance are described herein and include labeling substances known to those skilled in the art, such as fluorescent dyes, enzymes, coenzymes, chemiluminescent substances, and radioactive substances. Labeling further may include energy transfer between molecules in the hybridization complex by perturbation analysis, quenching, or electron transport between donor and acceptor molecules, the latter of which may be facilitated by double stranded match hybridization complexes. In the alternative, the fluorescent label may be a fluorescent bar code. Advantageously, the label may be light sensitive, wherein the label is light-activated and/or light cleaves the one or more linkers to release the molecular cargo.

The unique labels are, at least in part, nucleic acid in nature, and may be generated by sequentially attaching two or more detectable oligonucleotide tags to each other and each unique label may be associated with a separate agent. A detectable oligonucleotide tag may be an oligonucleotide that may be detected by sequencing of its nucleotide sequence and/or by detecting non-nucleic acid detectable moieties to which it may be attached. Oligonucleotide tags may be detectable by virtue of their nucleotide sequence, or by virtue of a non-nucleic acid detectable moiety that is attached to the oligonucleotide such as but not limited to a fluorophore, or by virtue of a combination of their nucleotide sequence and the non-nucleic acid detectable moiety. A detectable oligonucleotide tag may comprise one or more non-oligonucleotide detectable moieties. Examples of detectable moieties may include, but are not limited to, fluorophores, microparticles including quantum dots (Empodocles, et al., Nature 399:126-130, 1999), gold nanoparticles (Reichert et al., Anal. Chem. 72:6025-6029, 2000), microbeads (Lacoste et al., Proc. Natl. Acad. Sci. USA 97(17):9461-9466, 2000), biotin, DNP (dinitrophenyl), fucose, digoxigenin, haptens, and other detectable moieties known to those skilled in the art. In some embodiments, the detectable moieties may be quantum dots. Methods for detecting such moieties are described herein and/or are known in the art.

A unique label may be produced by sequentially attaching two or more detectable oligonucleotide tags to each other. The detectable tags may be present or provided in a plurality of detectable tags. The same or a different plurality of tags may be used as the source of each detectable tag may be part of a unique label. In other words, a plurality of tags may be subdivided into subsets and single subsets may be used as the source for each tag. One or more other species may be associated with the tags. In particular, nucleic acids released by a lysed cell may be ligated to one or more tags. These may include, for example, chromosomal DNA, RNA transcripts, tRNA, mRNA, mitochondrial DNA, or the like. Such nucleic acids may be sequenced, in addition to sequencing the tags themselves, which may yield information about the nucleic acid profile of the cells, which can be associated with the tags, or the conditions that the corresponding droplet or cell was exposed to.

### Embodiment 3: Methods of Using Solid Supports

A method of nucleic acid analysis is provided utilizing the solid support compositions and methods as taught herein. The method may comprise a variety of nucleic acid analyses, for example, the method can comprise single cell analysis, or the method comprises RNA analysis, DNA analysis, chromatin analysis or RNA-SEQ, ATAC PCR, processing an analyte comprising a protein, a peptide, an antibody, an organelle, a cell, a cellular fraction, processing a clinical sample, single cell microfluidics analysis or DROP-SEQ, single cell microwell array analysis or SEQ-WELL, or a single cell microwell platform analysis.

The invention accordingly may involve or be practiced as to high throughput and high resolution delivery of reagents to individual emulsion droplets that may contain cells, organelles, nucleic acids, proteins, etc. through the use of monodisperse aqueous droplets that are generated by a microfluidic device as a water-in-oil emulsion. The droplets are carried in a flowing oil phase and stabilized by a surfactant. In one aspect single cells or single organelles or single molecules (proteins, RNA, DNA) are encapsulated into uniform droplets from an aqueous solution/dispersion. In a related aspect, multiple cells or multiple molecules may take the place of single cells or single molecules. The aqueous droplets of volume ranging from 1 pL to 10 nL work as individual reactors. 10⁴ to 10⁵ single cells in droplets may be processed and analyzed in a single run. To utilize microdroplets for rapid large-scale chemical screening or complex biological library identification, different species of microdroplets, each containing the specific chemical compounds or biological probes cells or molecular barcodes of interest, have to be generated and combined at the preferred conditions, e.g., mixing ratio, concentration, and order of combination. Each species of droplet is introduced at a confluence point in a main microfluidic channel from separate inlet microfluidic channels. Preferably, droplet volumes are chosen by design such that one species is larger than others and moves at a different speed, usually slower than the other species, in the carrier fluid, as disclosed in U.S. Publication No. US 2007/0195127 and International Publication No. WO 2007/089541, each of which are incorporated herein by reference in their entirety. The channel width and length is selected such that faster species of droplets catch up to the slowest species. Size constraints of the channel prevent the faster moving droplets from passing the slower moving droplets resulting in a train of droplets entering a merge zone. Multi-step chemical reactions, biochemical reactions, or assay detection chemistries often require a fixed reaction time before species of different type are added to a reaction. Multi-step reactions are achieved by repeating the process multiple times with a second, third or more confluence points each with a separate merge point. Highly efficient and precise reactions and analysis of reactions are achieved when the frequencies of droplets from the inlet channels are matched to an optimized ratio and the volumes of the species are matched to provide optimized reaction conditions in the combined droplets. Fluidic droplets may be screened or sorted within a fluidic system of the invention by altering the flow of the liquid containing the droplets. For instance, in one set of embodiments, a fluidic droplet may be steered or sorted by directing the liquid surrounding the fluidic droplet into a first channel, a second channel, etc. In another set of embodiments, pressure within a fluidic system, for example, within different channels or within different portions of a channel, can be controlled to direct the flow of fluidic droplets. For example, a droplet can be directed toward a channel junction including multiple options for further direction of flow (e.g., directed toward a branch, or fork, in a channel defining optional downstream flow channels). Pressure within one or more of the optional downstream flow channels can be controlled to direct the droplet selectively into one of the channels, and changes in pressure can be effected on the order of the time required for successive droplets to reach the junction, such that the downstream flow path of each successive droplet can be independently controlled. In one arrangement, the expansion and/or contraction of liquid reservoirs may be used to steer or sort a fluidic droplet into a channel, e.g., by causing directed movement of the liquid containing the fluidic droplet. In another, the expansion and/or contraction of the liquid reservoir may be combined with other flow-controlling devices and methods, e.g., as described herein. Non-limiting examples of devices able to cause the expansion and/or contraction of a liquid reservoir include pistons. Key elements for using microfluidic channels to process droplets include: (1) producing droplet of the correct volume, (2) producing droplets at the correct frequency and (3) bringing together a first stream of sample droplets with a second stream of sample droplets in such a way that the frequency of the first stream of sample droplets matches the frequency of the second stream of sample droplets. Preferably, bringing together a stream of sample droplets with a stream of premade library droplets in such a way that the frequency of the library droplets matches the frequency of the sample droplets. Methods for producing droplets of a uniform volume at a regular frequency are well known in the art. One method is to generate droplets using hydrodynamic focusing of a dispersed phase fluid and immiscible carrier fluid, such as disclosed in U.S. Publication No. US 2005/0172476 and International Publication No. WO 2004/002627. It is desirable for one of the species introduced at the confluence to be a pre-made library of droplets where the library contains a plurality of reaction conditions, e.g., a library may contain plurality of different compounds at a range of concentrations encapsulated as separate library elements for screening their effect on cells or enzymes, alternatively a library could be composed of a plurality of different primer pairs encapsulated as different library elements for targeted amplification of a collection of loci, alternatively a library could contain a plurality of different antibody species encapsulated as different library elements to perform a plurality of binding assays. The introduction of a library of reaction conditions onto a substrate is achieved by pushing a premade collection of library droplets out of a vial with a drive fluid. The drive fluid is a continuous fluid. The drive fluid may comprise the same substance as the carrier fluid (e.g., a fluorocarbon oil). For example, if a library consists of ten pico-liter droplets is driven into an inlet channel on a microfluidic substrate with a drive fluid at a rate of 10,000 pico-liters per second, then nominally the frequency at which the droplets are expected to enter the confluence point is 1000 per second. However, in practice droplets pack with oil between them that slowly drains. Over time the carrier fluid drains from the library droplets and the number density of the droplets (number/mL) increases. Hence, a simple fixed rate of infusion for the drive fluid does not provide a uniform rate of introduction of the droplets into the microfluidic channel in the substrate. Moreover, library-to-library variations in the mean library droplet volume result in a shift in the frequency of droplet introduction at the confluence point. Thus, the lack of uniformity of droplets that results from sample variation and oil drainage provides another problem to be solved. For example if the nominal droplet volume is expected to be 10 pico-liters in the library, but varies from 9 to 11 pico-liters from library-to-library then a 10,000 pico-liter/second infusion rate will nominally produce a range in frequencies from 900 to 1,100 droplet per second. In short, sample to sample variation in the composition of dispersed phase for droplets made on chip, a tendency for the number density of library droplets to increase over time and library-to-library variations in mean droplet volume severely limit the extent to which frequencies of droplets may be reliably matched at a confluence by simply using fixed infusion rates. In addition, these limitations also have an impact on the extent to which volumes may be reproducibly combined. Combined with typical variations in pump flow rate precision and variations in channel dimensions, systems are severely limited without a means to compensate on a run-to-run basis. The foregoing facts not only illustrate a problem to be solved, but also demonstrate a need for a method of instantaneous regulation of microfluidic control over microdroplets within a microfluidic channel. Combinations of surfactant(s) and oils must be developed to facilitate generation, storage, and manipulation of droplets to maintain the unique chemical/biochemical/biological environment within each droplet of a diverse library. Therefore, the surfactant and oil combination must (1) stabilize droplets against uncontrolled coalescence during the drop forming process and subsequent collection and storage, (2) minimize transport of any droplet contents to the oil phase and/or between droplets, and (3) maintain chemical and biological inertness with contents of each droplet (e.g., no adsorption or reaction of encapsulated contents at the oil-water interface, and no adverse effects on biological or chemical constituents in the droplets). In addition to the requirements on the droplet library function and stability, the surfactant-in-oil solution must be coupled with the fluid physics and materials associated with the platform. Specifically, the oil solution must not swell, dissolve, or degrade the materials used to construct the microfluidic chip, and the physical properties of the oil (e.g., viscosity, boiling point, etc.) must be suited for the flow and operating conditions of the platform. Droplets formed in oil without surfactant are not stable to permit coalescence, so surfactants must be dissolved in the oil that is used as the continuous phase for the emulsion library. Surfactant molecules are amphiphilic—part of the molecule is oil soluble, and part of the molecule is water soluble. When a water-oil interface is formed at the nozzle of a microfluidic chip for example in the inlet module described herein, surfactant molecules that are dissolved in the oil phase adsorb to the interface. The hydrophilic portion of the molecule resides inside the droplet and the fluorophilic portion of the molecule decorates the exterior of the droplet. The surface tension of a droplet is reduced when the interface is populated with surfactant, so the stability of an emulsion is improved. In addition to stabilizing the droplets against coalescence, the surfactant should be inert to the contents of each droplet and the surfactant should not promote transport of encapsulated components to the oil or other droplets. A droplet library may be made up of a number of library elements that are pooled together in a single collection (see, e.g., US Patent Publication No. 2010002241). Libraries may vary in complexity from a single library element to 1015 library elements or more. Each library element may be one or more given components at a fixed concentration. The element may be, but is not limited to, cells, organelles, virus, bacteria, yeast, beads, amino acids, proteins, polypeptides, nucleic acids, polynucleotides or small molecule chemical compounds. The element may contain an identifier such as a label. The terms “droplet library” or “droplet libraries” are also referred to herein as an “emulsion library” or “emulsion libraries.” These terms are used interchangeably throughout the specification. A cell library element may include, but is not limited to, hybridomas, B-cells, primary cells, cultured cell lines, cancer cells, stem cells, cells obtained from tissue, or any other cell type. Cellular library elements are prepared by encapsulating a number of cells from one to hundreds of thousands in individual droplets. The number of cells encapsulated is usually given by Poisson statistics from the number density of cells and volume of the droplet. However, in some cases the number deviates from Poisson statistics as described in Edd et al., “Controlled encapsulation of single-cells into monodisperse picolitre drops.” Lab Chip, 8(8): 1262-1264, 2008. The discrete nature of cells allows for libraries to be prepared in mass with a plurality of cellular variants all present in a single starting media and then that media is broken up into individual droplet capsules that contain at most one cell. These individual droplets capsules are then combined or pooled to form a library consisting of unique library elements. Cell division subsequent to, or in some embodiments following, encapsulation produces a clonal library element. A bead based library element may contain one or more beads, of a given type and may also contain other reagents, such as antibodies, enzymes or other proteins. In the case where all library elements contain different types of beads, but the same surrounding media, the library elements may all be prepared from a single starting fluid or have a variety of starting fluids. In the case of cellular libraries prepared in mass from a collection of variants, such as genomically modified, yeast or bacteria cells, the library elements will be prepared from a variety of starting fluids. Often it is desirable to have exactly one cell per droplet with only a few droplets containing more than one cell when starting with a plurality of cells or yeast or bacteria, engineered to produce variants on a protein. In some cases, variations from Poisson statistics may be achieved to provide an enhanced loading of droplets such that there are more droplets with exactly one cell per droplet and few exceptions of empty droplets or droplets containing more than one cell. Examples of droplet libraries are collections of droplets that have different contents, ranging from beads, cells, small molecules, DNA, primers, antibodies. Smaller droplets may be in the order of femtoliter (fL) volume drops, which are especially contemplated with the droplet dispensors. The volume may range from about 5 to about 600 fL. The larger droplets range in size from roughly 0.5 micron to 500 micron in diameter, which corresponds to about 1 pico liter to 1 nano liter. However, droplets may be as small as 5 microns and as large as 500 microns. Preferably, the droplets are at less than 100 microns, about 1 micron to about 100 microns in diameter. The most preferred size is about 20 to 40 microns in diameter (10 to 100 picoliters). The preferred properties examined of droplet libraries include osmotic pressure balance, uniform size, and size ranges. The droplets within the emulsion libraries of the present invention may be contained within an immiscible oil which may comprise at least one fluorosurfactant. In some embodiments, the fluorosurfactant within the immiscible fluorocarbon oil may be a block copolymer consisting of one or more perfluorinated polyether (PFPE) blocks and one or more polyethylene glycol (PEG) blocks. In other embodiments, the fluorosurfactant is a triblock copolymer consisting of a PEG center block covalently bound to two PFPE blocks by amide linking groups. The presence of the fluorosurfactant (similar to uniform size of the droplets in the library) is critical to maintain the stability and integrity of the droplets and is also essential for the subsequent use of the droplets within the library for the various biological and chemical assays described herein. Fluids (e.g., aqueous fluids, immiscible oils, etc.) and other surfactants that may be utilized in the droplet libraries of the present invention are described in greater detail herein. The present invention can accordingly involve an emulsion library which may comprise a plurality of aqueous droplets within an immiscible oil (e.g., fluorocarbon oil) which may comprise at least one fluorosurfactant, wherein each droplet is uniform in size and may comprise the same aqueous fluid and may comprise a different library element. The present invention also provides a method for forming the emulsion library which may comprise providing a single aqueous fluid which may comprise different library elements, encapsulating each library element into an aqueous droplet within an immiscible fluorocarbon oil which may comprise at least one fluorosurfactant, wherein each droplet is uniform in size and may comprise the same aqueous fluid and may comprise a different library element, and pooling the aqueous droplets within an immiscible fluorocarbon oil which may comprise at least one fluorosurfactant, thereby forming an emulsion library. For example, in one type of emulsion library, all different types of elements (e.g., cells or beads), may be pooled in a single source contained in the same medium. After the initial pooling, the cells or beads are then encapsulated in droplets to generate a library of droplets wherein each droplet with a different type of bead or cell is a different library element. The dilution of the initial solution enables the encapsulation process. In some embodiments, the droplets formed will either contain a single cell or bead or will not contain anything, i.e., be empty. In other embodiments, the droplets formed will contain multiple copies of a library element. The cells or beads being encapsulated are generally variants on the same type of cell or bead. In another example, the emulsion library may comprise a plurality of aqueous droplets within an immiscible fluorocarbon oil, wherein a single molecule may be encapsulated, such that there is a single molecule contained within a droplet for every 20-60 droplets produced (e.g., 20, 25, 30, 35, 40, 45, 50, 55, 60 droplets, or any integer in between). Single molecules may be encapsulated by diluting the solution containing the molecules to such a low concentration that the encapsulation of single molecules is enabled. Formation of these libraries may rely on limiting dilutions.

The present invention also provides an emulsion library which may comprise at least a first aqueous droplet and at least a second aqueous droplet within an oil, in one embodiment a fluorocarbon oil, which may comprise at least one surfactant, in one embodiment a fluorosurfactant, wherein the at least first and the at least second droplets are uniform in size and comprise a different aqueous fluid and a different library element. The present invention also provides a method for forming the emulsion library which may comprise providing at least a first aqueous fluid which may comprise at least a first library of elements, providing at least a second aqueous fluid which may comprise at least a second library of elements, encapsulating each element of said at least first library into at least a first aqueous droplet within an immiscible fluorocarbon oil which may comprise at least one fluorosurfactant, encapsulating each element of said at least second library into at least a second aqueous droplet within an immiscible fluorocarbon oil which may comprise at least one fluorosurfactant, wherein the at least first and the at least second droplets are uniform in size and may comprise a different aqueous fluid and a different library element, and pooling the at least first aqueous droplet and the at least second aqueous droplet within an immiscible fluorocarbon oil which may comprise at least one fluorosurfactant thereby forming an emulsion library. One of skill in the art will recognize that methods and systems of the invention need not be limited to any particular type of sample, and methods and systems of the invention may be used with any type of organic, inorganic, or biological molecule (see, e.g, US Patent Publication No. 20120122714). In particular embodiments the sample may include nucleic acid target molecules. Nucleic acid molecules may be synthetic or derived from naturally occurring sources. In one embodiment, nucleic acid molecules may be isolated from a biological sample containing a variety of other components, such as proteins, lipids and non-template nucleic acids. Nucleic acid target molecules may be obtained from any cellular material, obtained from an animal, plant, bacterium, fungus, or any other cellular organism. In certain embodiments, the nucleic acid target molecules may be obtained from a single cell. Biological samples for use in the present invention may include viral particles or preparations. Nucleic acid target molecules may be obtained directly from an organism or from a biological sample obtained from an organism, e.g., from blood, urine, cerebrospinal fluid, seminal fluid, saliva, sputum, stool and tissue. Any tissue or body fluid specimen may be used as a source for nucleic acid for use in the invention. Nucleic acid target molecules may also be isolated from cultured cells, such as a primary cell culture or a cell line. The cells or tissues from which target nucleic acids are obtained may be infected with a virus or other intracellular pathogen. A sample may also be total RNA extracted from a biological specimen, a cDNA library, viral, or genomic DNA. Generally, nucleic acid may be extracted from a biological sample by a variety of techniques such as those described by Maniatis, et al., Molecular Cloning: A Laboratory Manual, Cold Spring Harbor, N.Y., pp. 280-281 (1982). Nucleic acid molecules may be single-stranded, double-stranded, or double-stranded with single-stranded regions (for example, stem- and loop-structures). Nucleic acid obtained from biological samples typically may be fragmented to produce suitable fragments for analysis. Target nucleic acids may be fragmented or sheared to desired length, using a variety of mechanical, chemical and/or enzymatic methods. DNA may be randomly sheared via sonication, e.g. Covaris method, brief exposure to a DNase, or using a mixture of one or more restriction enzymes, or a transposase or nicking enzyme. RNA may be fragmented by brief exposure to an RNase, heat plus magnesium, or by shearing. The RNA may be converted to cDNA. If fragmentation is employed, the RNA may be converted to cDNA before or after fragmentation. In one embodiment, nucleic acid from a biological sample is fragmented by sonication. In another embodiment, nucleic acid is fragmented by a hydroshear instrument. Generally, individual nucleic acid target molecules may be from about 40 bases to about 40 kb. Nucleic acid molecules may be single-stranded, double-stranded, or double-stranded with single-stranded regions (for example, stem- and loop-structures). A biological sample as described herein may be homogenized or fractionated in the presence of a detergent or surfactant. The concentration of the detergent in the buffer may be about 0.05% to about 10.0%. The concentration of the detergent may be up to an amount where the detergent remains soluble in the solution. In one embodiment, the concentration of the detergent is between 0.1% to about 2%. The detergent, particularly a mild one that is nondenaturing, may act to solubilize the sample. Detergents may be ionic or nonionic. Examples of nonionic detergents include triton, such as the Triton™ X series (Triton™ X-100 t-Oct-C6H4-(OCH2-CH2)xOH, x=9-10, Triton™ X-100R, Triton™ X-114 x=7-8), octyl glucoside, polyoxyethylene(9)dodecyl ether, digitonin, IGEPAL™ CA630 octylphenyl polyethylene glycol, n-octyl-beta-D-glucopyranoside (betaOG), n-dodecyl-beta, Tween™. 20 polyethylene glycol sorbitan monolaurate, Tween™ 80 polyethylene glycol sorbitan monooleate, polidocanol, n-dodecyl beta-D-maltoside (DDM), NP-40 nonylphenyl polyethylene glycol, C12E8 (octaethylene glycol n-dodecyl monoether), hexaethyleneglycol mono-n-tetradecyl ether (C14E06), octyl-beta-thioglucopyranoside (octyl thioglucoside, OTG), Emulgen, and polyoxyethylene 10 lauryl ether (C12E10). Examples of ionic detergents (anionic or cationic) include deoxycholate, sodium dodecyl sulfate (SDS), N-lauroylsarcosine, and cetyltrimethylammoniumbromide (CTAB). A zwitterionic reagent may also be used in the purification schemes of the present invention, such as Chaps, zwitterion 3-14, and 3-[(3-cholamidopropyl)dimethylammonio]-1-propanesulf-onate. It is contemplated also that urea may be added with or without another detergent or surfactant. Lysis or homogenization solutions may further contain other agents, such as reducing agents. Examples of such reducing agents include dithiothreitol (DTT), β-mercaptoethanol, DTE, GSH, cysteine, cysteamine, tricarboxyethyl phosphine (TCEP), or salts of sulfurous acid. Size selection of the nucleic acids may be performed to remove very short fragments or very long fragments. The nucleic acid fragments may be partitioned into fractions which may comprise a desired number of fragments using any suitable method known in the art. Suitable methods to limit the fragment size in each fragment are known in the art. In various embodiments of the invention, the fragment size is limited to between about 10 and about 100 Kb or longer. A sample in or as to the instant invention may include individual target proteins, protein complexes, proteins with translational modifications, and protein/nucleic acid complexes. Protein targets include peptides, and also include enzymes, hormones, structural components such as viral capsid proteins, and antibodies. Protein targets may be synthetic or derived from naturally-occurring sources. The invention protein targets may be isolated from biological samples containing a variety of other components including lipids, non-template nucleic acids, and nucleic acids. Protein targets may be obtained from an animal, bacterium, fungus, cellular organism, and single cells. Protein targets may be obtained directly from an organism or from a biological sample obtained from the organism, including bodily fluids such as blood, urine, cerebrospinal fluid, seminal fluid, saliva, sputum, stool and tissue. Protein targets may also be obtained from cell and tissue lysates and biochemical fractions. An individual protein is an isolated polypeptide chain. A protein complex includes two or polypeptide chains. Samples may include proteins with post translational modifications including but not limited to phosphorylation, methionine oxidation, deamidation, glycosylation, ubiquitination, carbamylation, s-carboxymethylation, acetylation, and methylation. Protein/nucleic acid complexes include cross-linked or stable protein-nucleic acid complexes. Extraction or isolation of individual proteins, protein complexes, proteins with translational modifications, and protein/nucleic acid complexes is performed using methods known in the art.

The invention can thus involve forming sample droplets. The droplets are aqueous droplets that are surrounded by an immiscible carrier fluid. Methods of forming such droplets are shown for example in Link et al. (U.S. patent application numbers 2008/0014589, 2008/0003142, and 2010/0137163), Stone et al. (U.S. Pat. No. 7,708,949 and U.S. patent application number 2010/0172803), Anderson et al. (U.S. Pat. No. 7,041,481 and which reissued as RE41,780) and European publication number EP2047910 to Raindance Technologies Inc. The content of each of which is incorporated by reference herein in its entirety. The present invention may relate to systems and methods for manipulating droplets within a high throughput microfluidic system. A microfluid droplet may encapsulate a differentiated cell, the cell is lysed and its mRNA is hybridized onto a capture bead containing barcoded oligo dT primers on the surface, all inside the droplet. The barcode is covalently attached to the capture bead via a flexible multi-atom linker like PEG. In a preferred embodiment, the droplets are broken by addition of a fluorosurfactant (like perfluorooctanol), washed, and collected. A reverse transcription (RT) reaction is then performed to convert each cell's mRNA into a first strand cDNA that is both uniquely barcoded and covalently linked to the mRNA capture bead. Subsequently, a universal primer via a template switching reaction is amended using conventional library preparation protocols to prepare an RNA-Seq library. Since all of the mRNA from any given cell is uniquely barcoded, a single library is sequenced and then computationally resolved to determine which mRNAs came from which cells. In this way, through a single sequencing run, tens of thousands (or more) of distinguishable transcriptomes can be simultaneously obtained. The oligonucleotide sequence may be generated on the bead surface. During these cycles, beads were removed from the synthesis column, pooled, and aliquoted into four equal portions by mass; these bead aliquots were then placed in a separate synthesis column and reacted with either dG, dC, dT, or dA phosphoramidite. In other instances, dinucleotide, trinucleotides, or oligonucleotides that are greater in length are used, in other instances, the oligo-dT tail is replaced by gene specific oligonucleotides to prime specific targets (singular or plural), random sequences of any length for the capture of all or specific RNAs. This process was repeated 12 times for a total of 4¹²=16,777,216 unique barcode sequences. Upon completion of these cycles, 8 cycles of degenerate oligonucleotide synthesis were performed on all the beads, followed by 30 cycles of dT addition. In other embodiments, the degenerate synthesis is omitted, shortened (less than 8 cycles), or extended (more than 8 cycles); in others, the 30 cycles of dT addition are replaced with gene specific primers (single target or many targets) or a degenerate sequence. The aforementioned microfluidic system is regarded as the reagent delivery system microfluidic library printer or droplet library printing system of the present invention. Droplets are formed as sample fluid flows from droplet generator which contains lysis reagent and barcodes through microfluidic outlet channel which contains oil, towards junction. Defined volumes of loaded reagent emulsion, corresponding to defined numbers of droplets, are dispensed on-demand into the flow stream of carrier fluid. The sample fluid may typically comprise an aqueous buffer solution, such as ultrapure water (e.g., 18 mega-ohm resistivity, obtained, for example by column chromatography), 10 mM Tris HCl and 1 mM EDTA (TE) buffer, phosphate buffer saline (PBS) or acetate buffer. Any liquid or buffer that is physiologically compatible with nucleic acid molecules can be used. The carrier fluid may include one that is immiscible with the sample fluid. The carrier fluid can be a non-polar solvent, decane (e.g., tetradecane or hexadecane), fluorocarbon oil, silicone oil, an inert oil such as hydrocarbon, or another oil (for example, mineral oil). The carrier fluid may contain one or more additives, such as agents which reduce surface tensions (surfactants). Surfactants can include Tween, Span, fluorosurfactants, and other agents that are soluble in oil relative to water. In some applications, performance is improved by adding a second surfactant to the sample fluid. Surfactants can aid in controlling or optimizing droplet size, flow and uniformity, for example by reducing the shear force needed to extrude or inject droplets into an intersecting channel. This can affect droplet volume and periodicity, or the rate or frequency at which droplets break off into an intersecting channel. Furthermore, the surfactant can serve to stabilize aqueous emulsions in fluorinated oils from coalescing. Droplets may be surrounded by a surfactant which stabilizes the droplets by reducing the surface tension at the aqueous oil interface. Preferred surfactants that may be added to the carrier fluid include, but are not limited to, surfactants such as sorbitan-based carboxylic acid esters (e.g., the “Span” surfactants, Fluka Chemika), including sorbitan monolaurate (Span 20), sorbitan monopalmitate (Span 40), sorbitan monostearate (Span 60) and sorbitan monooleate (Span 80), and perfluorinated polyethers (e.g., DuPont Krytox 157 FSL, FSM, and/or FSH). Other non-limiting examples of non-ionic surfactants which may be used include polyoxyethylenated alkylphenols (for example, nonyl-, p-dodecyl-, and dinonylphenols), polyoxyethylenated straight chain alcohols, polyoxyethylenated polyoxypropylene glycols, polyoxyethylenated mercaptans, long chain carboxylic acid esters (for example, glyceryl and polyglyceryl esters of natural fatty acids, propylene glycol, sorbitol, polyoxyethylenated sorbitol esters, polyoxyethylene glycol esters, etc.) and alkanolamines (e.g., diethanolamine-fatty acid condensates and isopropanolamine-fatty acid condensates). In some cases, an apparatus for creating a single-cell sequencing library via a microfluidic system provides for volume-driven flow, wherein constant volumes are injected over time. The pressure in fluidic cannels is a function of injection rate and channel dimensions. In one embodiment, the device provides an oil/surfactant inlet; an inlet for an analyte; a filter, an inlet for mRNA capture microbeads and lysis reagent; a carrier fluid channel which connects the inlets; a resistor; a constriction for droplet pinch-off; a mixer; and an outlet for drops. In an embodiment the invention provides apparatus for creating a single-cell sequencing library via a microfluidic system, which may comprise: an oil-surfactant inlet which may comprise a filter and a carrier fluid channel, wherein said carrier fluid channel may further comprise a resistor; an inlet for an analyte which may comprise a filter and a carrier fluid channel, wherein said carrier fluid channel may further comprise a resistor; an inlet for mRNA capture microbeads and lysis reagent which may comprise a filter and a carrier fluid channel, wherein said carrier fluid channel further may comprise a resistor; said carrier fluid channels have a carrier fluid flowing therein at an adjustable or predetermined flow rate; wherein each said carrier fluid channels merge at a junction; and said junction being connected to a mixer, which contains an outlet for drops. Accordingly, an apparatus for creating a single-cell sequencing library via a microfluidic system microfluidic flow scheme for single-cell RNA-seq is envisioned. Two channels, one carrying cell suspensions, and the other carrying uniquely barcoded mRNA capture bead, lysis buffer and library preparation reagents meet at a junction and is immediately co-encapsulated in an inert carrier oil, at the rate of one cell and one bead per drop. In each drop, using the bead's barcode tagged oligonucleotides as cDNA template, each mRNA is tagged with a unique, cell-specific identifier. The invention also encompasses use of a Drop-Seq library of a mixture of mouse and human cells. The carrier fluid may be caused to flow through the outlet channel so that the surfactant in the carrier fluid coats the channel walls. The fluorosurfactant can be prepared by reacting the perflourinated polyether DuPont Krytox 157 FSL, FSM, or FSH with aqueous ammonium hydroxide in a volatile fluorinated solvent. The solvent and residual water and ammonia can be removed with a rotary evaporator. The surfactant can then be dissolved (e.g., 2.5 wt %) in a fluorinated oil (e.g., Flourinert (3M)), which then serves as the carrier fluid. Activation of sample fluid reservoirs to produce regent droplets is based on the concept of dynamic reagent delivery (e.g., combinatorial barcoding) via an on-demand capability. The on-demand feature may be provided by one of a variety of technical capabilities for releasing delivery droplets to a primary droplet, as described herein.

From this disclosure and herein cited documents and knowledge in the art, it is within the ambit of the skilled person to develop flow rates, channel lengths, and channel geometries; and establish droplets containing random or specified reagent combinations can be generated on demand and merged with the “reaction chamber” droplets containing the samples/cells/substrates of interest. By incorporating a plurality of unique tags into the additional droplets and joining the tags to a solid support designed to be specific to the primary droplet, the conditions that the primary droplet is exposed to may be encoded and recorded. For example, nucleic acid tags can be sequentially ligated to create a sequence reflecting conditions and order of same. Alternatively, the tags can be added independently appended to solid support. Non-limiting examples of a dynamic labeling system that may be used to bioinformatically record information can be found at US Provisional Patent Application entitled “Compositions and Methods for Unique Labeling of Agents” filed Sep. 21, 2012 and Nov. 29, 2012. In this way, two or more droplets may be exposed to a variety of different conditions, where each time a droplet is exposed to a condition, a nucleic acid encoding the condition is added to the droplet each ligated together or to a unique solid support associated with the droplet such that, even if the droplets with different histories are later combined, the conditions of each of the droplets are remain available through the different nucleic acids. Non-limiting examples of methods to evaluate response to exposure to a plurality of conditions can be found at US Provisional Patent Application filed Sep. 21, 2012, and U.S. patent application Ser. No. 15/303874 filed Apr. 17, 2015 entitled “Systems and Methods for Droplet Tagging.” Accordingly, in or as to the invention it is envisioned that there can be the dynamic generation of molecular barcodes (e.g., DNA oligonucleotides, fluorophores, etc.) either independent from or in concert with the controlled delivery of various compounds of interest (drugs, small molecules, siRNA, CRISPR guide RNAs, reagents, etc.). For example, unique molecular barcodes can be created in one array of nozzles while individual compounds or combinations of compounds can be generated by another nozzle array. Barcodes/compounds of interest can then be merged with cell-containing droplets. An electronic record in the form of a computer log file is kept to associate the barcode delivered with the downstream reagent(s) delivered. This methodology makes it possible to efficiently screen a large population of cells for applications such as single-cell drug screening, controlled perturbation of regulatory pathways, etc. The device and techniques of the disclosed invention facilitate efforts to perform studies that require data resolution at the single cell (or single molecule) level and in a cost-effective manner. The invention envisions a high throughput and high-resolution delivery of reagents to individual emulsion droplets that may contain cells, nucleic acids, proteins, etc. through the use of monodisperse aqueous droplets that are generated one by one in a microfluidic chip as a water-in-oil emulsion.

Being able to dynamically track individual cells and droplet treatments/combinations during life cycle experiments, and having an ability to create a library of emulsion droplets on demand with the further capability of manipulating the droplets through the disclosed process(es) are advantageous. In the practice of the invention there can be dynamic tracking of the droplets and create a history of droplet deployment and application in a single cell based environment. Droplet generation and deployment is produced via a dynamic indexing strategy and in a controlled fashion in accordance with disclosed embodiments of the present invention. Microdroplets can be processed, analyzed and sorted at a highly efficient rate of several thousand droplets per second, providing a powerful platform which allows rapid screening of millions of distinct compounds, biological probes, proteins or cells either in cellular models of biological mechanisms of disease, or in biochemical, or pharmacological assays. A plurality of biological assays as well as biological synthesis are contemplated.

Polymerase chain reactions (PCR) are contemplated (see, e.g., US Patent Publication No. 20120219947). Methods of the invention may be used for merging sample fluids for conducting any type of chemical reaction or any type of biological assay. There may be merging sample fluids for conducting an amplification reaction in a droplet. Amplification refers to production of additional copies of a nucleic acid sequence and is generally carried out using polymerase chain reaction or other technologies well known in the art (e.g., Dieffenbach and Dveksler, PCR Primer, a Laboratory Manual, Cold Spring Harbor Press, Plainview, N.Y. [1995]). The amplification reaction may be any amplification reaction known in the art that amplifies nucleic acid molecules, such as polymerase chain reaction, nested polymerase chain reaction, polymerase chain reaction-single strand conformation polymorphism, ligase chain reaction (Barany F. (1991) PNAS 88:189-193; Barany F. (1991) PCR Methods and Applications 1:5-16), ligase detection reaction (Barany F. (1991) PNAS 88:189-193), strand displacement amplification and restriction fragments length polymorphism, transcription based amplification system, nucleic acid sequence-based amplification, rolling circle amplification, and hyper-branched rolling circle amplification. In certain embodiments, the amplification reaction is the polymerase chain reaction. Polymerase chain reaction (PCR) refers to methods by K. B. Mullis (U.S. Pat. Nos. 4,683,195 and 4,683,202, hereby incorporated by reference) for increasing concentration of a segment of a target sequence in a mixture of genomic DNA without cloning or purification. The process for amplifying the target sequence includes introducing an excess of oligonucleotide primers to a DNA mixture containing a desired target sequence, followed by a precise sequence of thermal cycling in the presence of a DNA polymerase. The primers are complementary to their respective strands of the double stranded target sequence. To effect amplification, primers are annealed to their complementary sequence within the target molecule. Following annealing, the primers are extended with a polymerase so as to form a new pair of complementary strands. The steps of denaturation, primer annealing and polymerase extension may be repeated many times (i.e., denaturation, annealing and extension constitute one cycle; there may be numerous cycles) to obtain a high concentration of an amplified segment of a desired target sequence. The length of the amplified segment of the desired target sequence is determined by relative positions of the primers with respect to each other, and therefore, this length is a controllable parameter. Methods for performing PCR in droplets are shown for example in Link et al. (U.S. Patent application numbers 2008/0014589, 2008/0003142, and 2010/0137163), Anderson et al. (U.S. Pat. No. 7,041,481 and which reissued as RE41,780) and European publication number EP2047910 to Raindance Technologies Inc. The content of each of which is incorporated by reference herein in its entirety.

The primer may be an oligonucleotide, whether occurring naturally as in a purified restriction digest or produced synthetically, which is capable of acting as a point of initiation of synthesis when placed under conditions in which synthesis of a primer extension product which is complementary to a nucleic acid strand is induced, (i.e., in the presence of nucleotides and an inducing agent such as DNA polymerase and at a suitable temperature and pH). The primer is preferably single stranded for maximum efficiency in amplification, but may alternatively be double stranded. If double stranded, the primer is first treated to separate its strands before being used to prepare extension products. Preferably, the primer is an oligodeoxy-ribonucleotide. The primer must be sufficiently long to prime the synthesis of extension products in the presence of the inducing agent. The exact lengths of the primers will depend on many factors, including temperature, source of primer and the use of the method.

The first sample fluid in certain embodiment contains nucleic acid templates. Droplets of the first sample fluid are formed as described above. Those droplets will include the nucleic acid templates. In certain embodiments, the droplets will include only a single nucleic acid template, and thus digital PCR may be conducted. The second sample fluid contains reagents for the PCR reaction. Such reagents generally include Taq polymerase, deoxynucleotides of type A, C, G and T, magnesium chloride, and forward and reverse primers, all suspended within an aqueous buffer. The second fluid also includes detectably labeled probes for detection of the amplified target nucleic acid, the details of which are discussed below. This type of partitioning of the reagents between the two sample fluids is not the only possibility. In some instances, the first sample fluid will include some or all of the reagents necessary for the PCR whereas the second sample fluid will contain the balance of the reagents necessary for the PCR together with the detection probes. Primers may be prepared by a variety of methods including but not limited to cloning of appropriate sequences and direct chemical synthesis using methods well known in the art (Narang et al., Methods Enzymol., 68:90 (1979); Brown et al., Methods Enzymol., 68:109 (1979)). Primers may also be obtained from commercial sources such as Operon Technologies, Amersham Pharmacia Biotech, Sigma, and Life Technologies. The primers may have an identical melting temperature. The lengths of the primers may be extended or shortened at the 5′ end or the 3′ end to produce primers with desired melting temperatures. Also, the annealing position of each primer pair may be designed such that the sequence and, length of the primer pairs yield the desired melting temperature. The simplest equation for determining the melting temperature of primers smaller than 25 base pairs is the Wallace Rule (Td=2(A+T)+4(G+C)). Computer programs may also be used to design primers, including but not limited to Array Designer Software (Arrayit Inc.), Oligonucleotide Probe Sequence Design Software for Genetic Analysis (Olympus Optical Co.), NetPrimer, and DNAsis from Hitachi Software Engineering. The TM (melting or annealing temperature) of each primer is calculated using software programs such as Oligo Design, available from Invitrogen Corp.

A droplet containing the nucleic acid is then caused to merge with the PCR reagents in the second fluid according to methods of the invention described above, producing a droplet that includes Taq polymerase, deoxynucleotides of type A, C, G and T, magnesium chloride, forward and reverse primers, detectably labeled probes, and the target nucleic acid. Once mixed droplets have been produced, the droplets are thermal cycled, resulting in amplification of the target nucleic acid in each droplet. Droplets may be flowed through a channel in a serpentine path between heating and cooling lines to amplify the nucleic acid in the droplet. The width and depth of the channel may be adjusted to set the residence time at each temperature, which may be controlled to anywhere between less than a second and minutes. The three temperature zones may be used for the amplification reaction. The three temperature zones are controlled to result in denaturation of double stranded nucleic acid (high temperature zone), annealing of primers (low temperature zones), and amplification of single stranded nucleic acid to produce double stranded nucleic acids (intermediate temperature zones). The temperatures within these zones fall within ranges well known in the art for conducting PCR reactions. See for example, Sambrook et al. (Molecular Cloning, A Laboratory Manual, 3rd edition, Cold Spring Harbor Laboratory Press, Cold Spring Harbor, N.Y., 2001). The three temperature zones can be controlled to have temperatures as follows: 95° C. (TH), 55° C. (TL), 72° C. (TM). The prepared sample droplets flow through the channel at a controlled rate. The sample droplets first pass the initial denaturation zone (TH) before thermal cycling. The initial preheat is an extended zone to ensure that nucleic acids within the sample droplet have denatured successfully before thermal cycling. The requirement for a preheat zone and the length of denaturation time required is dependent on the chemistry being used in the reaction. The samples pass into the high temperature zone, of approximately 95° C., where the sample is first separated into single stranded DNA in a process called denaturation. The sample then flows to the low temperature, of approximately 55° C., where the hybridization process takes place, during which the primers anneal to the complementary sequences of the sample. Finally, as the sample flows through the third medium temperature, of approximately 72° C., the polymerase process occurs when the primers are extended along the single strand of DNA with a thermostable enzyme. The nucleic acids undergo the same thermal cycling and chemical reaction as the droplets pass through each thermal cycle as they flow through the channel. The total number of cycles in the device is easily altered by an extension of thermal zones. The sample undergoes the same thermal cycling and chemical reaction as it passes through N amplification cycles of the complete thermal device. In other aspects, the temperature zones are controlled to achieve two individual temperature zones for a PCR reaction. In certain embodiments, the two temperature zones are controlled to have temperatures as follows: 95° C. (TH) and 60° C. (TL). The sample droplet optionally flows through an initial preheat zone before entering thermal cycling. The preheat zone may be important for some chemistry for activation and also to ensure that double stranded nucleic acid in the droplets is fully denatured before the thermal cycling reaction begins. In an exemplary embodiment, the preheat dwell length results in approximately 10 minutes preheat of the droplets at the higher temperature. The sample droplet continues into the high temperature zone, of approximately 95° C., where the sample is first separated into single stranded DNA in a process called denaturation. The sample then flows through the device to the low temperature zone, of approximately 60° C., where the hybridization process takes place, during which the primers anneal to the complementary sequences of the sample. Finally the polymerase process occurs when the primers are extended along the single strand of DNA with a thermostable enzyme. The sample undergoes the same thermal cycling and chemical reaction as it passes through each thermal cycle of the complete device. The total number of cycles in the device is easily altered by an extension of block length and tubing. After amplification, droplets may be flowed to a detection module for detection of amplification products. The droplets may be individually analyzed and detected using any methods known in the art, such as detecting for the presence or amount of a reporter. Generally, a detection module is in communication with one or more detection apparatuses. Detection apparatuses may be optical or electrical detectors or combinations thereof. Examples of suitable detection apparatuses include optical waveguides, microscopes, diodes, light stimulating devices, (e.g., lasers), photo multiplier tubes, and processors (e.g., computers and software), and combinations thereof, which cooperate to detect a signal representative of a characteristic, marker, or reporter, and to determine and direct the measurement or the sorting action at a sorting module. Further description of detection modules and methods of detecting amplification products in droplets are shown in Link et al. (U.S. patent application numbers 2008/0014589, 2008/0003142, and 2010/0137163) and European publication number EP2047910 to Raindance Technologies Inc. Examples of assays are also ELISA assays (see, e.g., US Patent Publication No. 20100022414). The present invention provides another emulsion library which may comprise a plurality of aqueous droplets within an immiscible fluorocarbon oil which may comprise at least one fluorosurfactant, wherein each droplet is uniform in size and may comprise at least a first antibody, and a single element linked to at least a second antibody, wherein said first and second antibodies are different. In one example, each library element may comprise a different bead, wherein each bead is attached to a number of antibodies and the bead is encapsulated within a droplet that contains a different antibody in solution. These antibodies may then be allowed to form “ELISA sandwiches,” which may be washed and prepared for a ELISA assay. Further, these contents of the droplets may be altered to be specific for the antibody contained therein to maximize the results of the assay. Single-cell assays are also contemplated as part of the present invention (see, e.g., Ryan et al., Biomicrofluidics 5, 021501 (2011) for an overview of applications of microfluidics to assay individual cells). A single-cell assay may be contemplated as an experiment that quantifies a function or property of an individual cell when the interactions of that cell with its environment may be controlled precisely or may be isolated from the function or property under examination. The research and development of single-cell assays is largely predicated on the notion that genetic variation causes disease and that small subpopulations of cells represent the origin of the disease. Methods of assaying compounds secreted from cells, subcellular components, cell-cell or cell-drug interactions as well as methods of patterning individual cells are also contemplated within the present invention.

Another aspect of the invention is the combination of the technologies described herein. For example, the use of a high-throughput single-cell RNA-Seq and/or targeted nucleic acid profiling (for example, sequencing, quantitative reverse transcription polymerase chain reaction, and the like) where the RNAs from different cells are tagged individually, allowing a single library to be created while retaining the cell identity of each read, as explained above. RNA-Seq profiling of single cells (e.g. single Th17 cells) may be performed on cells isolated in vivo (e.g. isolated directly from a subject/patient, preferably without further culture steps). RNA-Seq profiling of single cells may be performed on any number of cells, including tumor cells, associated infiltrating cells into a tumor, immune derived cells, microglia, astrocytes, CD4 cells, CD8 cells, most preferably Th17 cells. Computational analysis of the high-throughput single-cell RNA-Seq data allows, for example, to dissect the molecular basis of different functional cellular states. This also allows for selection of signature genes as described herein. Once selection of signature genes is performed, an optional further step is the validation of the signature genes using any number of technologies for knock-out or knock-in models. For example, as explained herein, mutations in cells and also mutated mice for use in or as to the invention can be by way of the CRISPR-Cas system or a Cas9-expressing eukaryotic cell or Cas-9 expressing eukaryote, such as a mouse.

Such a combination of technologies, e.g. in particular with direct isolation from the subject/patient, provides for more robust and more accurate data as compared to in vitro scenarios which cannot take into account the full in vivo system and networking. This combination, in several instances is thus more efficient, more specific, and faster. This combination provides for, for example, methods for identification of signature genes and validation methods of the same. Equally, screening platforms are provided for identification of effective therapeutics or diagnostics.

### Perturbation-Seq

In an embodiment of the invention, the functionalized solid support or a population of functionalized solid support described herein can be used in methods of measuring or determining or inferring RNA levels, e.g., massively parallel measuring or determining or inferring of RNA levels in a single cell or a cellular network or circuit in response to at least one perturbation parameter or advantageously a plurality of perturbation parameters or massively parallel perturbation parameters involving sequencing DNA of a perturbed cell, whereby RNA level and optionally protein level may be determined in the single cell in response to the at least one perturbation parameter or advantageously a plurality of perturbation parameters or massively parallel perturbation parameters. . The invention thus may involve a method of inferring or determining or measuring RNA in a single cell or a cellular network or circuit, e.g., massively parallel inferring or determining or measuring of RNA level in a single cell or a cellular network or circuit in response to at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98 ,99 or 100 or massively parallel perturbation parameter(s) comprising optionally so perturbing the cell or the cells or each cell of the cellular network or circuit with the perturbation parameter(s) and sequencing of the perturbed cell(s), whereby RNA level(s) and optionally protein level(s) is/are determined in the cell(s) in response to the perturbation parameter(s).

Perturb-seq, which combines single cell RNA-seq and CRISPR/Cas9 based perturbations identified by unique polyadenylated barcodes to perform many, tens of thousands in certain embodiments, of such assays in a single pooled experiment. By randomly integrating more than one sgRNA in each cell, Perturb-Seq is extended to test transcriptional phenotypes caused by genetic interactions. A computational framework, MIMOSCA (Multi-Input Multi-Output Single Cell Analysis) identifies the regulatory effects of individual perturbations and their combinations at different levels of resolution: from effects on each individual gene to functional signatures to proportional changes in cell types. Perturb-Seq accurately identifies known regulatory relations, and its individual gene target predictions can be validated by ChIP-Seq binding profiles. Using Perturb-Seq, genetic interactions including synergistic, buffering and dominant genetic interactions that could not be predicted from individual perturbations alone can be identified. Perturb-Seq can be flexibly applied to diverse cell metadata, to customize design and scope of pooled genomic assays. (See WO2017/075294, the content of which is incorporated herein).

### Drug Delivery

In one embodiment, the solid support or the population of solid support described herein can be used as a carrier or a nanocarrier for drug delivery. In some aspects, drug delivery includes DNA origami (see, e.g., Zhang et al., DNA Origami as an In Vivo Drug Delivery Vehicle for Cancer Therapy, ACS Nano, 2014, 8(7), pp 6633-6643), cellulose microparticles, and other polymer microparticle. The drug to be delivered can be a small-molecule drug reversibly attached to the solid support by a linkage analogous to, for example, Staben et al., Targeted drug delivery through the traceless release of tertiary and heteroayl amines from antibody-drug conjugates, Nature Chemistry (2016) 8:1112-1119; Gillies et al., Acetals as pH-sensitive linkages for drug delivery, Bioconjugate Chem., 2004, 15(6):1254-1263; or Paulick et al., Cleavable hydropholic linker for one-bead-one-compound sequencing of oligomer libraries ty tandem mass spectrometry, J. Comb. Chem. 2006, 8(3):417-426. In some aspects, the carrier or nanocarrier comprises a solid support having a spacer and a nucleic acid molecule. The nucleic acid molecule can be used as a capture probe for a drug of interest, e.g., a DNA or RNA binding protein, an oligonucleotide complementary or partially complementary to the capture probe.

### Use of RNA-Targeting Effector Protein in RNA or DNA Origami/In Vitro Assembly Lines Combinatorics

The functionalized solid support described herein can be used in RNA origami, which refers to nanoscale folded structures for creating two-dimensional or three-dimensional structures using RNA as integrated template. The folded structure is encoded in the RNA and the shape of the resulting RNA is thus determined by the synthesized RNA sequence (Geary, et al. 2014. Science, 345 (6198). pp. 799-804). The RNA origami may act as scaffold for arranging other components, such as proteins, into complexes. The RNA targeting effector protein of the invention can for instance be used to target proteins of interest to the RNA origami using a suitable guide RNA. These applications could also be applied in animal models for in vivo imaging of disease relevant applications or difficult-to culture cell types.

In some embodiments, the functionalized solid support can comprise an aptamer or a functional domain. An aptamer is a synthetic oligonucleotide that binds to a specific target molecule; for instance a nucleic acid molecule that has been engineered through repeated rounds of in vitro selection or SELEX (systematic evolution of ligands by exponential enrichment) to bind to various molecular targets such as small molecules, proteins, nucleic acids, and even cells, tissues and organisms. Aptamers are useful in that they offer molecular recognition properties that rival that of antibodies. In addition to their discriminate recognition, aptamers offer advantages over antibodies including that they elicit little or no immunogenicity in therapeutic applications. Accordingly, in the practice of the invention, either or both of the enzyme or the RNA can include a functional domain.

In another aspect, the invention provides a method for spatially patterning specific cells on surfaces (e.g. inorganic, organic, or biological). This is currently achievable (Todhunter et al. Nature Methods 2015), however Applicants' method has the specific advantage of enabling user-defined cellular placement that can be adjusted in real-time as opposed to pre-printing of oligonucleotides on surfaces. In an embodiment of this method, the cell functionalized barcode would be conjugated to cells using a non-specific, e.g., NETS-ester ligation or cholesterol (3′ cholesterol-TEG) or specific chemistry and the surface would be patterned with the cell functionalized probe (via photoactivation or another user-controlled activation scheme). By flowing the barcoded cells over the surface, some will be attached via the click (or other) functionalization paired on both molecules. In another embodiment of this method, the surface is patterned with the cell functionalized probe labeled with an oligonucleotide; cells can then be flowed over (streamed) and conjugated non-specifically (e.g. via NETS-ester ligation) or specifically. In an aspect of the invention, whole tissues or vibratome-sliced portions of biopsied human samples or whole mouse organ can be directly applied onto pre-barcoded surfaces. In another embodiment of this method the surface is patterned with the cell functionalized probe labeled with an oligonucleotide and cells labeled with complementary oligonucleotides can then be adhered to the surface in a specific manner.

Although the present invention and its advantages have been described in detail, it should be understood that various changes, substitutions and alterations can be made herein without departing from the spirit and scope of the invention as defined in the appended claims. The present invention will be further illustrated in the following Examples which are given for illustration purposes only and are not intended to limit the invention in any way.

### EXAMPLES

### Example 1 Methods

Activation of hydroxylated Beads Toyopearl beads (20 mL) made of methacrylic resin are washed sequentially with 20 mL of: 100% water, 75% water/acetonitrile (v/v), 50% water/acetonitrile (v/v), 25% water/acetonitrile (v/v), and dry acetonitrile (dried with 3 angstrom molecular sieves). The beads are added to a 100 mL flask along with dry acetonitrile (20 mL) and carbonyl diimidazole (CDI) (1.5 g, 9.25 mmol). The reaction is stirred at 400 rpm for 1 hour under nitrogen. After 1 hour, the beads are filtered and washed with 10 mL dry acetonitrile before being stored in dry acetonitrile at 4° C. Laboratory techniques can be found, for example, in Hermanson, et al. Biconjugate Techniques 3rd edition.

Synthesis of Thiolated Beads. After being stored overnight, a portion of the CDI-activated beads (5 mL) are warmed to room temperature, filtered by gravity and washed with dry acetonitrile. HS-PEG7.5k-NH₂ (5 mg/mL, ALDRICH #JKA5146) is dissolved in dry acetonitrile (5 mL) along with triethylamine (1.4 uL, 0.010 mmol). After being filtered and washed, the activated beads are added to the PEG solution. The reaction mixture is stirred overnight with a stir bar at 350rpm. The next day, the beads are filtered and washed with dry acetonitrile (50 mL) and stored in 10% EtOH/water (v/v).

Synthesis of Hydroxylated Beads. The CDI-activated beads (5 mL) prepared above are again warmed to room temperature, filtered, and washed with dry acetonitrile. HO-PEG1k-NH₂ (25 mg, CAS: 25322-68-3) is dissolved in dry acetonitrile (5 mL). The filtered beads are added to the flask with the PEG and the reaction is stirred overnight. After stirring overnight, the beads are filtered and washed with dry acetonitrile (50 mL) and stored in 10% EtOH/water (v/v).

Preparing Standard Curve with Phenyl PEG. To prepare beads with the phenyl-PEG-NH₂(CAS: 86770-76-5), the protocol for preparing thiolated beads is followed and the HS-PEG7.5k-NH₂ is replaced with phenyl-PEG-NH₂ (5 mg/mL). At the end of the synthesis, the filtrate is collected for UV-Vis spectroscopy. The concentration of PEG that had not reacted with the bead is determined using Beer's law.

Six solutions of phenyl-PEG-NH₂ (CAS:86770-76-5) are prepared in acetonitrile at the following concentrations: 20 mM, 10 mM, 2 mM, 1 mM, 500 uM, 250 uM. The absorbance of each solution is measured from 200-500 nm. The absorbance at 258 nm is used to determine the molar extinction coefficient.

### Quality Control Methods:

Bead digestion: Digest beads in DMSO with a base, e.g., KOH, NaOH, etc. Nucleotide bases will stay intact (though not annealed to one another) and read out the signatures on UV-Vis (longer wavelength than DMSO).

Linkage cleavage: A cleavable linkage can be added for quality control methods. Examples of linkages can include any photocleavable or acid-base labile linkage, e.g., linkage examples described herein for the spacer. A cleavable linkage can be added after the spacer molecule. For example, a disulfide linkage can be added after a PEG spacer, i.e., the order of attachment is bead-PEGspacer-(S-S)-oligonucleotides. Cleavage of this bond will allow us to look at the intact nucleotide sequence. This sequence can be run through a mass spectrometer or HPLC to get a clear picture. Alternatively, a cleavable linkage can be added on the bead side of the spacer, e.g., the disulfide linkage can be added on the bead side of the PEG or the oligonucleotide when nospacer is included, i.e., the order of attachment is bead-(S-S)-PEGspacer-oligonucleotides, or bead-(S—S)-oligonucleotides, to cleave either the PEG spacer with the sequence (after sequencing to look at both the sequence and the PEG), the sequence alone, or the PEG alone. Two linkages can be added to the functionalized solid support, i.e., a combination of the two methods above. For example, a linkage is added on the bead side of the spacer and after the spacer, i.e., the order of attachment is bead-linkage-spacer-linkage. The two linkages can be the same or different linkages and can be cleaved in the same reaction condition or different reaction conditions.

Having thus described in detail preferred embodiments of the present invention, it is to be understood that the invention defined by the above paragraphs is not to be limited to particular details set forth in the above description as many apparent variations thereof are possible without departing from the spirit or scope of the present invention.

1. A composition comprising a solid support or plurality of solid supports each solid support comprising one or more agents, and optionally a spacer.

2. The composition according to claim 2 wherein the solid support or plurality of solid supports comprises one or more beads or micro-bead or a plurality of micro-beads, micro-arrays, micro-wells, or micro-lids.

3. The composition of claim 2, wherein the solid support comprises a bead that is a silica bead, a hydrogel bead or a magnetic bead.

4. The method according to any one of claims 1-5, wherein the bead has a shape that is circular, square, star, or the bead is porous.

5. The composition according to claim 2, wherein the solid support comprises a magnetic core.

6. The composition of claim 1, wherein the solid support has an average particle size between about 10 microns to 200 microns, about 10 microns to 30 microns, about 30 microns to 50 microns, about 50 microns to100 microns, about 100 microns to 200 microns, or about 30 microns.

7. The composition according to any one of claims 1-9 wherein the bead or micro-bead has an average size, measured as average diameter of 20-40 μm.

8. The composition of claim 2, wherein the solid support comprises a polymer, optionally a hydroxylated methacrylic polymer, a hydroxylated poly(methyl methacrylate), a polystyrene polymer, a polypropylene polymer, a polyethylene polymer agarose, or cellulose.

9. The composition according to any one of claims 2-4, wherein the solid support comprises a spacer, the spacer comprising a polyethylene glycol polymer (PEG), a polysaccharide, an alkyl amine or a linker.

10. The composition according to claim 8, wherein the spacer comprises a PEG, the PEG is a hetero-functional PEG comprising two or more different functionalities, wherein at least one of the functionalities is a primary amine or a thiol.

11. The composition according to claim 9, wherein the thiol further comprises an acrydite moiety attached thereto.

12. The composition of claim 1, wherein the solid support comprises a spacer, the spacer comprising a polyethylene glycol polymer (PEG) having a molecular weight range of about 1,000 daltons to 8,000 daltons.

13. The composition of claim 12, wherein PEG has a molecular weight of about 1000 daltons, 2000 daltons, 3500 daltons, 5000 daltons, or 8000 daltons.

14. The composition according to claim 7, wherein the spacer comprises a photolabile linker, a fluoride ion labile linker, or a cleavable linker.

15. The composition of claim 8, wherein the spacer comprises a benzenesulfonylethyl linker, an o-nitrobenzyl carbonate photolabile linker, a 5-methoxy-2-nitrobenzyl carbonate photolabile linker, an o-nitrophenyl-1,3-propanediol base photolabile linker, a fluoride ion labile diisopropylsilyl linker, a fluoride ion labile disiloxyl phosphoramidite linker, a NPE carbonate linker, a 9-fluorenylmethyl linker, a phthaloyl linker, an oxalyl linker, a malonic acid linker, a succinyl linker, a diglycolic acid linker, a hydroquinone-O,O′-diacetic acid (Q-linker), or a thiophospate linker.

16. The composition of claim 14, wherein the spacer comprises a benzenesulfonylethyl linker cleavable with triethylamine/dioxane.

17. The composition of claim 14, wherein the spacer comprises a nonyl phenol ethoxylate (NPE) carbonate linker cleavable with 1,8-Diazabicyclo[5.4.0]undec-7-ene (DBU)/pyridine.

18. The composition of claim 14, wherein the spacer comprises a 9-fluorenylmethyl linker or a phthaloyl linker cleavable with DBU.

19. The composition of claim 1, wherein the spacer comprises a succinic acid linked to an N-methylglycine (sarcosine) derivatized support, a succinic acid linked to 1,6-bis methylaminohexane spacer, a succinic acid linked to N-propyl polyethylene glycol Tentagel support, or a succinyl-sarcosine linkage.

20. The composition according to claim 1, wherein the spacer is grafted onto the solid support via an amine linkage, a secondary amine linkage, a thioether linkage, an ether linkage, a carbamate linkage, or an amide linkage.

21. The composition of claim 1, wherein the one or more agents comprises one or more of oligonucleotides, nucleotides, analogs thereof; a molecular barcode; a Unique Molecular Identifier; a oligodT; an amplification primer; a cell type specific sequence; a pathogen-specific sequence, or a TCR specific sequence, and surface reactive nucleic acid molecule(s).

22. The composition of claim 1, wherein the one or more agents comprises nucleic acid sequences of an In-silico Polymerase Chain Reaction (ISPCR) Primer, a Barcode, a Unique Molecular Identifier (UMI) and a Universal Sequence.

23. The composition of claim 1 wherein the one or more agents are nucleic acid molecules in a 3′ to 5′ orientation.

24. The composition according to any one of claims 1-7 wherein the one or more agents are nucleic acid molecules in a 5′ to 3′ orientation.

25. A kit comprising:
a solid support having a surface bearing reacting groups;
one or more activator(s) selected from 2-fluoro-1-methylpyridinium (FMP), carbonyl diimidazole (CDI) and a tosyl compound (Ts);
a spacer compound; and
optionally one or more nucleic acid molecules.

26. A method for functionalizing a surface of a solid support, comprising:
a) reacting a solid support having surface bearing reacting groups with an activator, so as to obtain a solid support with an activated surface comprising an activating moiety,
b) reacting the activated surface with a spacer compound having a first moiety that reacts with the activating moiety and optionally a second moiety comprising a functional group whereby the reacting of this step b) obtains, on the solid support, a spacer grafted-thereon, whereby the surface of the solid support is functionalized.

27. The method of claim 26, wherein reacting of step b) obtains, on the solid support, a spacer grafted-thereon having the second moiety comprising the functional group exposed for reaction.

28. The method of claim 26 or claim 27, wherein the step a) reacting is under conditions comprising dry conditions or non-aqueous conditions or solid phase synthesis conditions.

29. The method according to any one of claims 26 to 28, wherein the solid support comprises a hydrogel bead or a magnetic bead.

30. The method according any one of claims 26-28, wherein the solid support is a silica bead.

31. The method according to any one of claims 26-30, wherein the bead has a shape that is circular, square, star, or the bead is porous.

32. The method according to any of claims 26 to 31, wherein the activator comprises 2-Fluoro-1-Methylpyridinium (FMP), Carbonyl Diimidazole (CDI), bis-epoxide, divinylsulfone, cyanogen bromide, or an organic sulfonyl halide, optionally wherein the organic sulfonyl halide comprises tosyl chloride or tresyl chloride.

33. The method according to claim 32, wherein the activating moiety comprises a tosyl group, imidazolyl carbamate group, or methylpyridinium group.

34. The method according to claim 26 or claim 27, wherein the reacting groups on the surface of the solid support comprise a hydroxyl, a carboxyl, a thiol, an amine, a diol, or a combination thereof.

35. The method according to any one of claims 26 to 37, wherein the solid support comprises a polymer, optionally wherein the polymer is hydroxylated methacrylic polymer or hydroxylated poly(methyl methacrylate), polystyrene polymer, polypropylene polymer, polyethylene polymer, agarose, or cellulose.

36. The method of any one of claims 26 to 35, wherein the solid support has an average particle size ranging between about 10 microns to 200 microns, about 10 microns to 30 microns, about 30 microns to 50 microns, about 50 microns to 100 microns, about 100 microns to 200 microns, or about 30 microns

37. The method according to any one of claims 26 to 36, wherein the spacer comprises a polyethylene glycol polymer (PEG), a polysaccharide, an alkyl amine, or a succinyl linker.

38. The method according to any one of claims 26 to 37, wherein the spacer comprises a photolabile linker, a fluoride ion labile linker, or a cleavable linker.

39. The method according to any one of claims 26 to 38, wherein the spacer comprises a benzenesulfonylethyl linker, an o-nitrobenzyl carbonate photolabile linker, a 5-methoxy-2-nitrobenzyl carbonate photolabile linker, an o-nitrophenyl-1,3-propanediol base photolabile linker, a fluoride ion labile diisopropylsilyl linker, a fluoride ion labile disiloxyl phosphoramidite linker, a NPE carbonate linker, a 9-fluorenylmethyl linker, a phthaloyl linker, an oxalyl linker, a malonic acid linker, a diglycolic acid linker, a hydroquinone-O,O′-diacetic acid (Q-linker), or a thiophospate linker.

40. The method of claim 39, wherein the spacer comprises a benzenesulfonylethyl linker cleavable with triethylamine/dioxane, a nonyl phenol ethoxylate (NPE) carbonate linker cleavable with 1,8-Diazabicyclo[5.4.0]undec-7-ene (DBU)/pyridine, or a 9-fluorenylmethyl linker or a phthaloyl linker cleavable with DBU.

41. The method according to any one of claims 26 to 39, wherein the spacer comprises a succinic acid linked to an N-methylglycine (sarcosine) derivatized support, a succinic acid linked to 1,6-bis methylaminohexane spacer, a succinic acid linked to N-propyl polyethylene glycol Tentagel support, or a succinyl-sarcosine linkage.

42. The method according to any one of claims 26 to 39, wherein the spacer comprises a polyethylene glycol polymer (PEG) having a molecular weight range of about 1,000 daltons to 8,000 daltons, about 1000 daltons, 2000 daltons, 3500 daltons, 5000 daltons, or 8000 daltons.

43. The method according to any one of claims 26 to 42, wherein the functional group exposed for reaction comprises a thiol group, a disulfide linkage, a hydroxyl group, or a phenyl group.

44. The method according to any one of claims 26 to 43, wherein the first moiety comprises an amine, an amide, a thiol, a carboxyl, or a hydroxyl group.

45. The method according to any one of claims 26 to 44, wherein the spacer is grafted onto the solid support via an amine linkage, a secondary amine linkage, a thioether linkage, an ether linkage, a disulfide linkage, or an amide linkage.

46. The method according to any one of claims 26 to 45, wherein the first moiety comprises NH₂, the second moiety comprises SH, OH or phenyl, and the spacer comprises a PEG; or the first moiety-spacer-second moiety comprises: X—(Y)ₙ—Z, wherein X is a thiol, a hydroxyl, an amine, or a carboxyl, Y is PEG or a methylene group, and Z is a thiol, a hydroxyl, an amine, or a carboxyl, and wherein n is an integer between 1 to 30.

47. A method for preparing a population of functionalized solid support comprising a surface reactive nucleic acid molecule comprising:
a′) reacting a functionalized solid support, optionally comprising a spacer, having a functional group exposed for reaction with a nucleic acid molecule so as to obtain a solid support comprising a surface reactive nucleic acid molecule.

48. A method for preparing a population of functionalized solid support comprising surface reactive nucleic acid molecules in sequence(s) comprising:
a″) reacting a solid support comprising a surface reactive nucleic acid molecule with another nucleic acid molecule so as to obtain a solid support comprising surface reactive nucleic acid molecules in sequence(s).

49. The method according to claim 48, wherein the functionalized solid support comprises a spacer, the spacer having a functional group exposed for reaction is prepared by a method comprising
a) reacting a solid support having surface bearing reacting groups with an activator, so as to obtain a solid support with an activated surface comprising an activating moiety,
b) reacting the activated surface with a spacer compound having a first moiety that reacts with the activating moiety and optionally a second moiety comprising a functional group whereby the reacting of this step b) obtains, on the solid support, a spacer grafted-thereon, whereby the surface of the solid support is functionalized.

50. The method according to claim 48 wherein step a″ is repeated n times, wherein n is an integer between 1 and 100.

51. The method according to claim 48 wherein step a″ is repeated so as to obtain surface reactive nucleic acid molecules in sequences of an ISPCR Primer, a Barcode, a Unique Molecular Identifier (UMI) and a Universal Sequence; or so that the surface reactive nucleic acid molecules comprise one or more of the following:
oligonucleotides, nucleotides, analogs thereof,
a molecular barcode,
a Unique Molecular Identifier,
a oligodT,
an amplification primer,
a cell type specific sequence,
a pathogen-specific sequence, or
a TCR specific sequence.

52. The method according to claim 48, wherein the nucleic acid molecules are in a 3′ to 5′ orientation, or wherein the nucleic acid molecules are in a 5′ to 3′ orientation.

53. The method according to any one of the preceding claims 48 to 52, wherein the solid support comprises a bead or a plurality of beads, or a micro-bead or a plurality of micro-beads, micro-arrays, micro-wells, or micro-lids.

54. The method according to claim 53, wherein the bead or micro-bead has an average size, measured as average diameter of 20-40 μm.

55. The method according to any one of the preceding claims wherein the solid support comprises a magnetic core.

56. The method of claim 48, performed as to a plurality of solid supports.

57. The method according to any of the preceding claims further comprising reacting the spacer with an acrydite, whereby the acrydite has the functional group exposed for reaction.

58. A solid support or a population or solid supports or one or more beads or micro-bead or a population of micro-beads, micro-arrays, micro-wells, or micro-lids prepared by a method of claim 26 or claim 48.

59. A method for nucleic acid analysis:
wherein the method comprises single cell analysis, or
the method comprises RNA analysis, DNA analysis, chromatin analysis or RNA-SEQ, or
the method comprises ATAC PCR, or
the method comprises processing an analyte comprising a protein, a peptide, an antibody, an organelle, a cell, a cellular fraction, or
the method is for processing a clinical sample, or
the method comprises single cell microfluidics analysis or DROP-SEQ, or
the method comprises a single cell microwell array analysis or SEQ-WELL, or
the method comprises a single cell microwell platform analysis
wherein the method comprises use of a solid support or plurality of solid supports of claim 26-57; or solid support or plurality of solid supports or one or more beads or micro-bead or a plurality of micro-beads, micro-arrays, micro-wells, or micro-lids of any one of claims 1-24.


SEQUENCE LISTING (5 sequences)

SEQ ID NO: 1 - DNA, 14 bp, Artificial Sequence
  Synthetic oligonucleotide
  misc_feature (1)..(1): terminal phosphate
  misc_feature (1)..(14): sequence consists of two dimerized DNA chains
  misc_feature (8)..(8): terminal phosphate
ggattccggattcc

SEQ ID NO: 2 - DNA, 16 bp, Artificial Sequence
  Synthetic Oligonucleotide
  misc_feature (1)..(16): sequence consists of two dimerized DNA chains
  misc_feature (8)..(8): terminal phosphate
  misc_feature (16)..(16): terminal phosphate
ggaattccggaattcc

SEQ ID NO: 3 - DNA, 16 bp, Artificial Sequence
  Synthetic Oligonucleotide
  misc_feature (1)..(1): terminal phosphate
  misc_feature (1)..(16): sequence consists of two dimerized DNA chains
  misc_feature (8)..(8): terminal phosphate
  misc_feature (9)..(9): terminal phosphate
  misc_feature (16)..(16): terminal phosphate
ggaattccggaattcc

SEQ ID NO: 4 - DNA, 8 bp, Artificial Sequence
  Synthetic oligonucleotide
  misc_feature (1)..(6): n = any nucleotide
  misc_feature (7)..(8): v = not t, not u
  misc_feature (8)..(8): 3′ polydT modification
nnnnnnvv

SEQ ID NO: 5 - DNA, 10 bp, Artificial Sequence
  Synthetic oligonucleotide
  misc_feature (1)..(6): n = any nucleotide
  misc_feature (7)..(8): v = not t, not u
nnnnnnvvtt